(19) United States
(12) Patent Application Publication
(10) Pub. No.: US 2020/0216514 A1
(43) Pub. Date: Jul. 9, 2020

(54) Generalized Extracellular Molecule Sensor

(71) Applicant: ETH ZURICH, Zurich (CH)

(72) Inventors: Martin Fussenegger, Magenwil (CH); Leo Scheller, Basel (CH); Tobias Strittmatter, Basel (CH); David W. Fuchs, Lorrach (DE); Daniel Bojar, Basel (CH)

(21) Appl. No.: 16/737,076

(22) Filed: Jan. 8, 2020

Related U.S. Application Data
(60) Provisional application No. 62/790,361, filed on Jan. 9, 2019.

(51) Int. Cl. C07K 14/705; C12N 15/63

(52) U.S. Cl. CPC C07K 14/705; C12N 15/63; A61K 38/00; C07K 2319/03; C12N 2510/00; C07K 2317/24

(57) ABSTRACT

Provided herein are generalized extracellular molecule sensors (GEMSs) and polynucleotides encoding the GEMSs. Also provided herein are methods of making and using the GEMSs, such as therapeutic and diagnostic methods.

### CROSS REFERENCE TO RELATED APPLICATIONS

This application claims the benefit of and priority to U.S. Provisional Application No. 62/790,361, filed Jan. 9, 2019, which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Jan. 6, 2020, is named STB-009US_SL.txt and is 62,122 bytes in size.

### BACKGROUND

Mammalian cells programmed to respond to extracellular inputs in a predictable manner have become increasingly important for a wide range of applications, such as cancer immunotherapy, tissue patterning and smart cell implants. The field of programmable receptor engineering is rapidly evolving (See Lim W A & June C H, Cell, 2017, 168:724-740; Brenner M et al., Nat. Chem. Biol., 2017, 13:131-132), but robust sensing of soluble molecules still mostly relies on natural receptors that can be rewired to drive expression of transgenes that have a desired biological function. For example, natural ligand-receptor interactions have been used to engineer designer cells that sense various biomarkers and secrete therapeutic peptides in response. This approach has been used to develop therapeutic cell implants consisting of encapsulated designer cells for the detection and treatment of psoriasis, Graves' disease and metabolic syndrome (See Schukur L et al., Sci. Transl. Med., 2015, 7:318ra201; Saxena P et al., Proc. Natl. Acad. Sci. USA, 2016, 113:1244-1249; Ye H et al., Proc. Natl. Acad. Sci. USA, 2013 110:141-146). Nevertheless, engineering robust input-output relationships in mammalian cells is a laborious iterative process, and many molecules that would be valuable targets for diagnostic or therapeutic purposes do not oftentimes bind to any known naturally occurring receptor. Thus, large groups of potential molecular inputs cannot be readily targeted by this approach. Notably, this includes many synthetic small-molecule compounds, intracellular proteins and extracellular proteins without known signaling function(s).

### SUMMARY

Provided herein are chimeric ligand receptors and related methods.

In one aspect, provided herein is a chimeric ligand receptor comprising a receptor subunit, wherein the receptor subunit comprises a scaffold domain; wherein the scaffold domain comprises an extracellular domain and a transmembrane domain; wherein the extracellular domain is operably linked to a ligand binding domain; wherein the transmembrane domain is operably linked to an intracellular signaling domain; wherein the receptor subunit multimerizes via its scaffold domain in the presence of one or more additional receptor subunits; and wherein the multimerized receptor subunits undergo a conformational reorganization upon ligand binding to the chimeric ligand receptor.

In some embodiments, the chimeric ligand receptor further comprises one or more additional receptor subunits, wherein each additional receptor subunit comprises a scaffold domain; wherein the scaffold domain of each additional receptor subunit comprises an extracellular domain and a transmembrane domain; wherein the extracellular domain of each additional receptor subunit is operably linked to a ligand binding domain; and wherein the transmembrane domain of each additional receptor subunit is operably linked to an intracellular signaling domain. In some embodiments, the receptor subunits multimerize via their scaffold domains to form the chimeric ligand receptor. In some embodiments, the multimerized receptor subunits undergo a conformational reorganization upon ligand binding to the chimeric receptor. In some embodiments, the chimeric ligand binding domains of each receptor subunit bind the same ligand.

In another aspect, provided herein is a chimeric ligand receptor comprising two or more receptor subunits, wherein each receptor subunit comprises a scaffold domain; wherein the receptor subunits multimerize via their scaffold domains to form the chimeric ligand receptor; wherein each scaffold domain comprises an extracellular domain and a transmembrane domain; wherein the extracellular domain is operably linked to a ligand binding domain, wherein the ligand binding domains of each receptor subunit bind the same ligand; wherein the transmembrane domain is operably linked to an intracellular signaling domain; and wherein binding of the ligand induces a conformational reorganization of the multimerized receptor subunits.

In some embodiments, multimerization of the receptor subunits occurs prior to ligand binding. In some embodiments, the multimerized receptor subunits comprise a dimer, a trimer, tetramer, pentamer, or hexamer. In some embodiments, the multimerized receptor subunits comprise a dimer. In some embodiments, the conformational reorganization comprises a rotation of each scaffold domain around its own axis. In some embodiments, the conformational reorganization activates the intracellular signaling domains of each receptor subunit.

In some embodiments, the scaffold domain comprises the extracellular domain and transmembrane domain of a cytokine receptor. In some embodiments, the scaffold domain comprises the extracellular domain and transmembrane domain of an erythropoietin receptor (EpoR). In some embodiments, the scaffold domain is inert to its native ligand. In some embodiments, the scaffold domain is inert to erythropoietin. In some embodiments, the scaffold domain comprises an extracellular domain and transmembrane domain comprising an amino acid sequence having 90% or greater sequence identity to SEQ ID NO: 8.

In some embodiments, the extracellular domain, the transmembrane domain, or both the extracellular domain and transmembrane domain of the scaffold domain comprise one or more modifications. In some embodiments, the extracellular domain comprises one or more amino acid substitutions, optionally wherein the extracellular domain comprises an F93A amino acid substitution. In some embodiments, one or more additional amino acid residues are inserted adjacent to the transmembrane domain. In some embodiments, one or more additional amino acid residues are inserted within the transmembrane domain. In some embodiments, the one or more additional amino acid residues are alanine residues. In some embodiments, the transmembrane domain further comprises one, two, three, or four additional alanine residues. In some embodiments, the one or more additional amino acid residues are inserted C-terminal to the transmembrane domain.

In some embodiments, the ligand binding domain is linked to the extracellular domain through an extracellular linker region. In some embodiments, the extracellular linker region comprises one or more amino acid residues, optionally wherein the one or more amino acid residues comprise amino acids residues Serine-Glycine-Glutamic acid-Phenylalanine (SEQ ID NO: 26).

In some embodiments, the ligand binding domain does not bind a native ligand of the scaffold domain. In some embodiments, the ligand binding domain does not bind erythropoietin. In some embodiments, the ligand binding domain is not derived from a cytokine receptor.

In some embodiments, the ligand binding domain binds to a soluble ligand selected from the group consisting of a protein complex, a protein, a peptide, a nucleic acid, a small molecule, and a chemical agent. In some embodiments, the soluble ligand is selected from the group consisting of an antigen, a cytokine, a survival factor, a chemokine, a hormone, a transmitter, a growth factor, extracellular matrix, and a death factor. In some embodiments, the ligand binding domain binds to caffeine. In some embodiments, the ligand binding domain binds to an antigen.

In some embodiments, the ligand binding domain comprises an antibody, or antigen-binding fragment thereof. In some embodiments, the ligand binding domain comprises a single chain variable fragment (scFv), or a single-domain antibody (sdAb). In some embodiments, each of the ligand binding domains comprises a single chain variable fragment (scFv), optionally wherein each scFv specifically binds to a distinct epitope of the antigen. In some embodiments, the ligand binding domains of each receptor subunit are distinct from one another. In some embodiments, the chimeric ligand receptor comprises two ligand binding domains, and wherein one ligand binding domain comprises an immunoglobulin heavy chain variable domain (V_{H}) and the second ligand binding domain comprises an immunoglobulin light chain variable domain (V_{L}). In some embodiments, the ligand binding domains of each receptor subunit are the same as one another. In some embodiments, the antibody, or antigen-binding fragment thereof, is a single-domain VHH camelid antibody that homodimerizes in the presence of caffeine.

In some embodiments, the intracellular signaling domain is inert to native ligand binding of the scaffold domain. In some embodiments, the intracellular signaling domain does not comprise an endogenous intracellular signaling domain of the scaffold domain. In some embodiments, the intracellular signaling domain does not comprise an endogenous erythropoietin receptor (EpoR) intracellular signaling domain. In some embodiments, the intracellular signaling domain does not comprise an endogenous cytokine receptor intracellular signaling domain.

In some embodiments, the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway. In some embodiments, the intracellular signaling domain is selected from the group consisting of an intracellular signal transduction domain of IL-6RB (interleukin 6 receptor B), an intracellular signal transduction domain of FGFR1 (fibroblast growth factor receptor 1), and an intracellular signal transduction domain of VEGFR2 (vascular endothelial growth factor receptor 2). In some embodiments, the intracellular signaling domain is an intracellular signal transduction domain of IL-6RB and induces downstream signaling via the JAK/STAT signaling pathway. In some embodiments, the intracellular signaling domain is an intracellular signal transduction domain of FGFR1 and induces downstream signaling via the MAPK signaling pathway. In some embodiments, the intracellular signaling domain is an intracellular signal transduction domain of VEGFR2 and induces downstream signaling via the PLCG signaling pathway. In some embodiments, the intracellular signaling domain is an intracellular signal transduction domain of VEGFR2 and induces downstream signaling via the PI3K/Akt signaling pathways.

In some embodiments, the intracellular signaling domain comprises one or more modifications that modulate signaling activity of the intracellular signaling domain, optionally wherein the one or more modifications are one or more amino acid substitutions.

In some embodiments, the scaffold domain comprises the extracellular domain and transmembrane domain of an erythropoietin receptor (EpoR) or an extracellular domain and transmembrane domain comprising an amino acid sequence having 90% or greater sequence identity to SEQ ID NO: 8, wherein the extracellular domain comprises an F93A amino acid substitution, wherein the scaffold domain is inert to erythropoietin; wherein the ligand binding domain does not bind erythropoietin; and wherein the intracellular signaling domain does not comprise an endogenous erythropoietin receptor (EpoR) intracellular signaling domain. In some embodiments, the ligand binding domain binds to caffeine. In some embodiments, the ligand binding domain comprises a single-domain VHH camelid antibody that homodimerizes in the presence of caffeine. In some embodiments, the ligand binding domain binds rapamycin, RR120, nicotine, SunTag, or PSA (prostate-specific antigen). In some embodiments, the ligand binding domain comprises an antibody or antigen-binding fragment targeting rapamycin, RR120, nicotine, SunTag, or PSA (prostate-specific antigen). In some embodiments, the intracellular signaling domain comprises an intracellular signal transduction domain of IL-6RB (interleukin 6 receptor B), an intracellular signal transduction domain of FGFR1 (fibroblast growth factor receptor 1), or an intracellular signal transduction domain of VEGFR2 (vascular endothelial growth factor receptor 2). In some embodiments, the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway. In some embodiments, the intracellular signaling domain comprises an intracellular signal transduction domain of IL-6RB (interleukin 6 receptor B). In some embodiments, the intracellular signaling domain induces downstream signaling via a JAK/STAT (Janus kinase/signal transducer and activator of transcription) signaling pathway.

In another aspect, provided herein is an isolated polynucleotide or a set of isolated polynucleotides encoding the chimeric ligand receptor. In some embodiments, the isolated polynucleotide or set of isolated polynucleotides comprises the cDNA of the chimeric ligand receptor.

In another aspect, provided herein is an isolated polynucleotide or a set of isolated polynucleotides comprising a nucleic acid sequence encoding a chimeric ligand receptor subunit, wherein the receptor subunit comprises a scaffold domain capable of multimerizing the receptor subunit in the presence of one or more additional receptor subunits and wherein the receptor subunit is capable of undergoing a conformational reorganization induced by ligand binding when multimerized; wherein the scaffold domain comprises an extracellular domain and a transmembrane domain; wherein the extracellular domain is operably linked to a ligand binding domain; and wherein the transmembrane domain is operably linked to an intracellular signaling domain.

In some embodiments, the isolated polynucleotide or set of isolated polynucleotides further comprises one or more nucleic acid sequences, wherein each additional nucleic acid sequence encoding an additional chimeric ligand receptor subunit; wherein each additional receptor subunit comprises a scaffold domain; wherein the scaffold domain of each additional receptor subunit comprises an extracellular domain and a transmembrane domain; wherein the extracellular domain of each additional receptor subunit is operably linked to a ligand binding domain; and wherein the transmembrane domain of each additional receptor subunit is operably linked to an intracellular signaling domain. In some embodiments, the receptor subunits multimerize via their scaffold domains to form the chimeric ligand receptor. In some embodiments, the multimerized receptor subunits undergo a conformational reorganization upon ligand binding to the chimeric receptor. In some embodiments, the chimeric ligand binding domains of each receptor subunit bind the same ligand.

In another aspect, provided herein is an isolated polynucleotide or a set of isolated polynucleotides comprising two or more nucleic acid sequences, wherein each nucleic acid sequence encoding a chimeric ligand receptor subunit; wherein each receptor subunit comprises a scaffold domain; wherein the receptor subunits multimerize via their scaffold domains to form the chimeric ligand receptor; wherein each scaffold domain comprises an extracellular domain and a transmembrane domain; wherein the extracellular domain is operably linked to a ligand binding domain, wherein the ligand binding domains of each receptor subunit bind the same ligand; wherein the transmembrane domain is operably linked to an intracellular signaling domain; and wherein binding of the ligand induces a conformational reorganization of the multimerized receptor subunits.

In some embodiments, multimerization of the receptor subunits occurs prior to ligand binding. In some embodiments, the multimerized receptor subunits comprise a dimer, a trimer, tetramer, pentamer, or hexamer. In some embodiments, the multimerized receptor subunits comprise a dimer. In some embodiments, the conformational reorganization comprises a rotation of each scaffold domain around its own axis. In some embodiments, the conformational reorganization activates the intracellular signaling domains of each receptor subunit

In some embodiments, the scaffold domain comprises the extracellular domain and transmembrane domain of a cytokine receptor. In some embodiments, the scaffold domain comprises the extracellular domain and transmembrane domain of an erythropoietin receptor (EpoR). In some embodiments, the scaffold domain is inert to its native ligand. In some embodiments, the scaffold domain is inert to erythropoietin. In some embodiments, the scaffold domain comprises an extracellular domain and transmembrane domain comprising an amino acid sequence having 90% or greater sequence identity to SEQ ID NO: 8.

In some embodiments, the extracellular domain, the transmembrane domain, or both the extracellular domain and transmembrane domain of the scaffold domain comprise one or more modifications. In some embodiments, the extracellular domain comprises one or more amino acid substitutions, optionally wherein the extracellular domain comprises an F93A amino acid substitution. In some embodiments, one or more additional amino acid residues are inserted adjacent to the transmembrane domain. In some embodiments, one or more additional amino acid residues are inserted within the transmembrane domain. In some embodiments, the one or more additional amino acid residues are alanine residues. In some embodiments, the transmembrane domain further comprises one, two, three, or four additional alanine residues. In some embodiments, the one or more additional amino acid residues are inserted C-terminal to the transmembrane domain.

In some embodiments, the ligand binding domain is linked to the extracellular domain through an extracellular linker region. In some embodiments, the extracellular linker region comprises one or more amino acid residues, optionally wherein the one or more amino acid residues comprise amino acids residues Serine-Glycine-Glutamic acid-Phenylalanine (SEQ ID NO: 26).

In some embodiments, the ligand binding domain does not bind a native ligand of the scaffold domain. In some embodiments, the ligand binding domain does not bind erythropoietin. In some embodiments, the ligand binding domain is not derived from a cytokine receptor.

In some embodiments, the ligand binding domain binds to a soluble ligand selected from the group consisting of a protein complex, a protein, a peptide, a nucleic acid, a small molecule, and a chemical agent. In some embodiments, the soluble ligand is selected from the group consisting of an antigen, a cytokine, a survival factor, a chemokine, a hormone, a transmitter, a growth factor, extracellular matrix, and a death factor. In some embodiments, the ligand binding domain binds to caffeine. In some embodiments, the ligand binding domain binds to an antigen.

In some embodiments, the ligand binding domain comprises an antibody, or antigen-binding fragment thereof. In some embodiments, the ligand binding domain comprises a single chain variable fragment (scFv), or a single-domain antibody (sdAb). In some embodiments, each of the ligand binding domains comprises a single chain variable fragment (scFv), optionally wherein each scFv specifically binds to a distinct epitope of the antigen. In some embodiments, the ligand binding domains of each receptor subunit are distinct from one another. In some embodiments, the chimeric ligand receptor comprises two ligand binding domains, and wherein one ligand binding domain comprises an immunoglobulin heavy chain variable domain (V_{H}) and the second ligand binding domain comprises an immunoglobulin light chain variable domain (V_{L}). In some embodiments, the ligand binding domains of each receptor subunit are the same as one another. In some embodiments, the antibody, or antigen-binding fragment thereof, is a single-domain VHH camelid antibody that homodimerizes in the presence of caffeine.

In some embodiments, the intracellular signaling domain is inert to native ligand binding of the scaffold domain. In some embodiments, the intracellular signaling domain does not comprise an endogenous intracellular signaling domain of the scaffold domain. In some embodiments, the intracellular signaling domain does not comprise an endogenous erythropoietin receptor (EpoR) intracellular signaling domain. In some embodiments, the intracellular signaling domain does not comprise an endogenous cytokine receptor intracellular signaling domain.

In some embodiments, the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway. In some embodiments, the intracellular signaling domain is selected from the group consisting of an intracellular signal transduction domain of IL-6RB (interleukin 6 receptor B), an intracellular signal transduction domain of FGFR1 (fibroblast growth factor receptor 1), and an intracellular signal transduction domain of VEGFR2 (vascular endothelial growth factor receptor 2). In some embodiments, the intracellular signaling domain is an intracellular signal transduction domain of IL-6RB and induces downstream signaling via the JAK/STAT signaling pathway. In some embodiments, the intracellular signaling domain is an intracellular signal transduction domain of FGFR1 and induces downstream signaling via the MAPK signaling pathway. In some embodiments, the intracellular signaling domain is an intracellular signal transduction domain of VEGFR2 and induces downstream signaling via the PLCG signaling pathway. In some embodiments, the intracellular signaling domain is an intracellular signal transduction domain of VEGFR2 and induces downstream signaling via the PI3K/Akt signaling pathways.

In some embodiments, the intracellular signaling domain comprises one or more modifications that modulate signaling activity of the intracellular signaling domain, optionally wherein the one or more modifications are one or more amino acid substitutions.

In some embodiments, the scaffold domain comprises the extracellular domain and transmembrane domain of an erythropoietin receptor (EpoR) or an extracellular domain and transmembrane domain comprising an amino acid sequence having 90% or greater sequence identity to SEQ ID NO: 8, wherein the extracellular domain comprises an F93A amino acid substitution, wherein the scaffold domain is inert to erythropoietin; wherein the ligand binding domain does not bind erythropoietin; and wherein the intracellular signaling domain does not comprise an endogenous erythropoietin receptor (EpoR) intracellular signaling domain. In some embodiments, the ligand binding domain binds to caffeine. In some embodiments, the ligand binding domain comprises a single-domain VHH camelid antibody that homodimerizes in the presence of caffeine. In some embodiments, the ligand binding domain binds rapamycin, RR120, nicotine, SunTag, or PSA (prostate-specific antigen). In some embodiments, the ligand binding domain comprises an antibody or antigen-binding fragment targeting rapamycin, RR120, nicotine, SunTag, or PSA (prostate-specific antigen). In some embodiments, the intracellular signaling domain comprises an intracellular signal transduction domain of IL-6RB (interleukin 6 receptor B), an intracellular signal transduction domain of FGFR1 (fibroblast growth factor receptor 1), or an intracellular signal transduction domain of VEGFR2 (vascular endothelial growth factor receptor 2). In some embodiments, the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway. In some embodiments, the intracellular signaling domain comprises an intracellular signal transduction domain of IL-6RB (interleukin 6 receptor B). In some embodiments, the intracellular signaling domain induces downstream signaling via a JAK/STAT (Janus kinase/signal transducer and activator of transcription) signaling pathway.

In another aspect, provided herein is a vector or a set of vectors comprising the polynucleotide or set of polynucleotides.

In another aspect, provided herein is a genetically engineered cell comprising the polynucleotide or set of polynucleotides or the vector or set of vectors.

In another aspect, provided herein is a genetically engineered cell expressing the chimeric ligand receptor.

In some embodiments, the cell further comprises an engineered transgene, wherein the transgene comprises a synthetic promoter operably linked to a polynucleotide comprising a nucleic acid sequence encoding a target product. In some embodiments, the synthetic promoter is responsive to intracellular signaling from the chimeric ligand receptor. In some embodiments, the target product is selected from the group consisting of a therapeutic molecule, a prophylactic molecule, and a diagnostic molecule. In some embodiments, the target product is glucagon-like peptide 1.

In some embodiments, the cell further expresses one or more additional chimeric ligand receptors. In some embodiments, the chimeric ligand receptors are each distinct from one another. In some embodiments, the ligand binding domains of each chimeric ligand receptor bind a different soluble ligand.

In another aspect, provided herein is a genetically engineered cell expressing two or more chimeric ligand receptors. In some embodiments, the chimeric ligand receptors are each distinct from one another. In some embodiments, the ligand binding domains of each chimeric ligand receptor bind a different soluble ligand. In some embodiments, the cell further comprises two or more engineered transgenes, wherein each transgene comprises a synthetic promoter operably linked to a polynucleotide comprising a nucleic acid sequence encoding a target product. In some embodiments, each synthetic promoter is responsive to intracellular signaling from a distinct chimeric ligand receptor from the two or more chimeric ligand receptors expressed on the cell. In some embodiments, each target product is independently selected from the group consisting of a therapeutic molecule, a prophylactic molecule, and a diagnostic molecule.

In some embodiments, the cell is a mammalian cell. In some embodiments, the mammalian cell is a stem cell or a neuronal cell. In some embodiments, the stem cell is selected from the group consisting of an adult stem cell, an iPS cell, a bone marrow stem cell, a peripheral blood stem cell, and a mesenchymal stem cell (MSC). In some embodiments, the mammalian cell is an immune cell. In some embodiments, the immune cell is selected from the group consisting of a T cell, a B cell, an NK cell, and a dendritic cell. In some embodiments, the immune cell is a T cell. In some embodiments, the T cell is selected from the group consisting of a helper T cell, a cytotoxic T cell, a memory T cell, a regulatory T cell, a natural killer T cell, and a gamma delta T cell.

In another aspect, provided herein is a method comprising contacting the chimeric ligand receptor or the genetically engineered cell with a biological tissue or biological fluid. In some embodiments, the biological tissue or biological fluid is in a subject or is obtained from a subject. In some embodiments, the subject has been diagnosed with, is at risk of developing, or is suspected of having a medical condition, optionally wherein the medical condition is a cancer or inflammatory condition.

In another aspect, provided herein is a method of activating a signaling pathway. The method comprises contacting the chimeric ligand receptor or the genetically engineered cell with a cognate ligand under conditions suitable for the chimeric ligand receptor to bind the cognate ligand, wherein binding of the cognate ligand with the chimeric ligand receptor induces a conformational reorganization of the multimerized scaffold domains that activates the intracellular signaling domains. In some embodiments, the method further comprises administering the cognate ligand to a surface of a cell.

In another aspect, provided herein is a method of producing a genetically engineered cell expressing a chimeric ligand receptor. The method comprises synthesizing a chimeric ligand receptor expression vector encoding a chimeric ligand receptor comprising a scaffold domain capable of multimerizing and comprising an extracellular domain and a transmembrane domain, a ligand binding domain operably linked to the extracellular binding domain of the scaffold domain, and an intracellular signaling domain operably linked to the transmembrane domain of the scaffold domain, by fusing a first nucleic acid encoding the ligand binding domain to a second nucleic acid encoding the scaffold domain, and fusing the second nucleic acid with a third nucleic acid encoding the intracellular signaling domain; transfecting the chimeric ligand receptor expression vector into a cell; and inducing expression of the chimeric ligand receptor in the cell.

In another aspect, provided herein is a method of producing a genetically engineered cell expressing a chimeric ligand receptor. The method comprises transfecting the isolated polynucleotide or set of isolated polynucleotides or the vector or set of vectors into a cell; and inducing expression of the chimeric ligand receptor in the cell. In some embodiments, the method further comprises transfecting into the cell an isolated polynucleotide comprising a synthetic promoter operably linked to a nucleic acid sequence encoding a target product. In some embodiments, the synthetic promoter is responsive to intracellular signaling from the chimeric ligand receptor. In some embodiments, inducing expression of the chimeric ligand receptor comprises culturing the cell under conditions suitable for the cell to express the chimeric ligand receptor on a cell membrane of the cell.

In another aspect, provided herein is a method comprising transfecting a cell with the polynucleotide or set of isolated polynucleotides or vector or set of vectors, optionally wherein the cell is a mammalian cell, and optionally wherein the mammalian cell is an immune cell.

In another aspect, provided herein is a kit comprising a polynucleotide comprising a nucleic acid sequence encoding at least one chimeric ligand receptor. In some embodiments, the kit further comprises at least one engineered transgene comprising a synthetic promoter operably linked to a polynucleotide comprising a nucleic acid sequence encoding a target product.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features, aspects, and advantages of the present disclosure will become better understood with regard to the following description, and accompanying drawings.

FIG. 1 shows a schematic of the mechanism of receptor activation. In the absence of input, the EpoR is locked in an inactive conformation. Binding of ligand facilitates interaction of intracellular domains for downstream signaling.

FIG. 2 shows a schematic of the generalized extracellular molecule sensor (GEMS) platform. The input molecules rapamycin, RR120, nicotine, SunTag and PSA cover a wide range of molecular weights and were chosen as inputs to verify the generality of the GEMS platform. The affinity domains dimerize by different mechanisms to activate the receptor. Rapamycin-induced FRB/FKBP heterodimerization was used for initial characterization of the system. The camelid heavy chain antibody VHH_{A52} forms homodimers in the presence of RR120. The variable chains of the nicotine antibody Nic12 were fused separately in the receptor framework for heterodimeric receptors based on nicotine-induced stabilization of heavy and light chain interactions. The anti-GCN4 scFv can bind epitopes of a SunTag for homodimeric receptors. Two different scFvs bind to distinct epitopes of PSA for heterodimeric receptors. Extracellular and transmembrane domains of EpoR cluster as preformed dimers that inhibit downstream signaling in the absence of ligand but can be activated by the dimerization of extracellular domains. Ligand-induced changes of the orientation of intracellular signal transduction domains were used to activate four different dimerization-dependent signaling pathways. Black arrows show the main signaling pathways of indicated GEMS devices. Dashed arrows exemplify possible activation of additional signaling pathways. All pathways have been rewired for transgene expression. A STAT3, NF-κB or NFAT responsive minimal promoter is used for readout of JAK/STAT, PI3K/Akt or PLCG signaling, respectively. At the end of the MAPK signaling cascade, ERK phosphorylates a TetR-Elk1 fusion protein that activates transcription from a reporter plasmid. Exchange of the reporter protein SEAP for expression of other proteins could be used in various applications, such as therapeutic peptide expression in response to disease markers.

FIG. 3A shows that introducing alanine residues C-terminally of the transmembrane domain elongates the α-helix and results in a rotation of about 100°. The ‘off’ state of the receptor can still allow a JAK interaction, depending on the number of alanine residues. The variant with three alanine residues is pictured to assume an ‘off’ conformation with low JAK interaction and an ‘on’ conformation with high JAK interaction, whereas the variant without alanine residues leads to a JAK interaction in the ‘off’ state. FIG. 3A discloses SEQ ID NO: 28 as “AAAA”. FIG. 3B shows that rapamycin induced gene expression by different receptor scaffolds, labeled 0 to 4 to indicate the number of alanine residues added between the EpoR transmembrane domain and the IL-6RB intracellular domain. The variant with three alanine residues shows relatively low absolute values for reporter gene expression but the highest signal-to-noise ratio. Graph shows the mean and data points of n=3 biologically independent samples and is representative of three independent experiments. Numbers above the bars indicate fold changes of reporter concentration in the supernatant after 24 h induction, calculated by dividing reporter concentration in the presence of inducer by reporter concentration in the absence of inducer.

FIG. 4A shows the molecular structure of RR120. VHH_{A52} binds to one half of the symmetric molecule, leading to dimerization of two antibodies. FIG. 4B shows that the RR120-induced SEAP expression increases with decreasing amount of plasmid (pLeo615) for P_{hCMV}-driven receptor expression. Graph shows the mean as a bar diagram overlaid with a dot plot of individual data points of n=3 biologically independent samples and are representative of three independent experiments.

FIG. 5A shows the effect of mutations in IL6RB. Introducing the mutation Y759A in the intracellular domain (pLeo618) increases the signal-to-noise ratio as well as absolute values of SEAP expression. Numbers above the bars indicate fold changes of reporter concentration in the supernatant after 24 h induction, calculated by dividing reporter concentration in the presence of inducer by reporter concentration in the absence of inducer. FIG. 5B shows the effect of mutations in EpoR. GEMS_{RR120} (pLeo619) is induced with RR120 but not with erythropoietin. Erythropoietin also does not interfere with RR120 induced signaling. Receptors without F93A modification (pLeo618) respond strongly to erythropoietin. Graphs show the mean as a bar diagram overlaid with a dot plot of individual data points of n=3 biologically independent samples and are representative of three independent experiments. FIG. 5C shows that GEMS_{RR120} provides higher total SEAP expression than nonoptimized constructs, and the signal-to-noise ratio is improved to over 40. Exchanging the intracellular domain of IL-6RB to the intracellular domain of FGFR1 (pLeo628) decreases the signal-to-noise ratio but results in more sensitive receptors. Graph shows the mean±s.d. as a bar diagram overlaid with a dot plot of individual data points of n=9 biologically independent samples. ****P<0.0001. Numbers above the bars indicate fold changes of reporter concentration in the supernatant after 24 h induction, calculated by dividing reporter concentration in the presence of inducer by reporter concentration in the absence of inducer.

FIG. 6A shows that adding alanine residues between the EpoR transmembrane domain and FGFR1 intracellular domain (pLeo628, pLeo642, pLeo643, pLeo644, pLeo645) has only minor effects on signal-to-noise ratios. The variant without alanine residues was chosen for further experiments. FIG. 6B shows that a PIP-Elk1 (pAT13) and a PIP reporter (pMF199) present lower absolute expression levels but a similar dynamic range compared to the TetR-Elk1 fusion (MKp37) and the TetR reporter (pMF111), confirming the modularity of this setup. Graphs show the mean as a bar diagram overlaid with a dot plot of individual data points of n=3 biologically independent samples and are representative of three independent experiments.

FIG. 7A shows that in HEK-293 cells, MAPK can be induced by bFGF. Overexpression of FGFR1 (pLeo698) increases reporter gene expression. FIG. 7B shows STAT3 can be induced with IL-6. Overexpression of IL-6RA (pLeo696) and/or IL6-RB (pLeo697) increases reporter gene expression. Graphs show the mean as a bar diagram overlaid with a dot plot of individual data points of n=3 biologically independent samples and are representative of three independent experiments.

FIG. 8 shows that VEGFR-GEMS_{RR120} signals via multiple pathways, activating reporters specific for NFAT, MAPK and NF-κB signaling. Graph show the mean±s.d. as a bar diagram overlaid with a dot plot of individual data points of n=9 biologically independent samples. ****P<0.0001. Numbers above the bars indicate fold changes of reporter concentration in the supernatant after 24 h induction, calculated by dividing reporter concentration in the presence of inducer by reporter concentration in the absence of inducer.

FIG. 9 shows that GEMS_{nicotine} (pLeo626/pLeo627, pLeo667/pLeo668) is inducible with nicotine concentrations typically reached after smoking. One cigarette is considered to generate blood nicotine concentrations of 100 nM. At this point, fold changes for SEAP expression are 1.6 for JAK/STAT-GEMS_{nicotine} and 2.5 for MAPK-GEMS_{nicotine}. Graphs show the mean±s.d. as a bar diagram overlaid with a dot plot of individual data points of n=9 biologically independent samples. ****P<0.0001. Numbers above the bars indicate fold changes of reporter concentration in the supernatant after 24 h induction, calculated by dividing reporter concentration in the presence of inducer by reporter concentration in the absence of inducer.

FIG. 10A shows that crude lysate of bacteria producing SunTagged mCherry induces SEAP expression in HEK cells equipped with GEMS_{SunTag} (pLeo620, pLeo669). The volume of SunTag containing bacterial lysate added for induction is indicated as a percentage of culture medium volume. Graphs show the mean±s.d. as a bar diagram overlaid with a dot plot of individual data points of n=9 biologically independent samples. ****P<0.0001. Numbers above the bars indicate fold changes of reporter concentration in the supernatant after 24 h induction, calculated by dividing reporter concentration in the presence of inducer by reporter concentration in the absence of inducer. FIG. 10B shows bacterial crude lysate containing SunTag-mCherry induces GEMS_{SunTag}, but not GEMS_{RR120}. RR120 induces GEMS_{RR120}, but not GEMS_{SunTag}. These controls verify that reporter induction is specific to the designed inducer receptor pair. The graph shows the mean as a bar diagram overlaid with a dot plot of individual data points of n=3 biologically independent samples and is representative of three independent experiments.

FIG. 11A shows that JAK/STAT-GEMS_{PSA} (pLeo622/pLeo623) can distinguish diagnostically important differences in PSA concentration between 1 and 20 ng/mL with high significance. FIG. 11B shows that MAPK-GEMS_{PSA} (pLeo670/pLeo671) can distinguish PSA concentrations between 0.1 and 4 ng/mL. FIG. 11C shows the profiling of PSA levels in patient serum using MAPK-GEMS_{PSA}. MAPK-GEMS_{PSA} cells were cultivated with 10% serum from prostate cancer patients (patients 1-3) or PSA-negative control serum (from patients that underwent radical prostatectomy). The culture medium containing PSA-negative control serum was spiked with different amounts of PSA to reach final concentrations of 0-10 ng/mL, with 0 ng/mL as the negative control. Because of the 1:10 serum dilution in culture medium, a spiked PSA concentration of 1 ng/mL corresponds to 10 ng/mL PSA in patient serum. SEAP expression was profiled from the culture supernatant. ELISA-based quantification confirmed the correlation of PSA levels in the patient samples (patient 1: 1 ng/mL; patient 2: 5 ng/mL; patient 3: 20 ng/mL) with GEMS output performance. Graphs show the mean±s.d. as a bar diagram overlaid with a dot plot of individual data points of n=9 biologically independent samples. ***P<0.001, ****P<0.0001. Numbers above the bars indicate fold changes of reporter concentration in the supernatant after 24 h induction, calculated by dividing mean reporter concentration in the presence of inducer by mean reporter concentration in the absence of inducer or by reporter concentration for lower inducer concentrations as specified.

FIG. 12 shows the dose-response curve of GEMS_{PSA}. MAPK-GEMS_{PSA} signal in a concentration range that is diagnostically relevant to detect biochemical recurrence after radical prostatectomy, while JAK/STAT-GEMS_{PSA} exhibits an almost linear dose response over the PSA concentration range between 1 to 20 ng/mL that is critical for screening. PSA concentrations below 2 ng/mL are considered physiological. Concentrations between 2 to 4 ng/mL indicate a need for further monitoring. Concentrations between 4 to 10 ng/mL are considered to be a diagnostic grey zone and a prostate biopsy may be recommended, depending on additional factors. Concentrations above 10 ng/mL are indicative of a high risk of prostate cancer. Dose response curves were generated as four-parameter dose-response curve with GraphPad Prism 7.

FIG. 13A shows that mutations of the PLCG or STAT binding sites (PLCG:Y766F, STAT:Y677F) in the intracellular domain of FGFR1 have no observable effect on MAPK signaling. FIG. 13B shows that MAPK-GEMS had only a minor effect on NFAT and STAT3. Mutation of the PLCG or STAT binding sites decreased the respective activation of NFAT or STAT3. JAK/STAT-GEMS without Y759A had only a minor effect on MAPK signaling. Graphs show the mean as a bar diagram overlaid with a dot plot of individual data points of n=3 biologically independent samples and are representative of three independent experiments. FIG. 13C shows the results of GEMS multiplexing. JAK/STAT-GEMS_{SunTag} and MAPK-GEMS_{RR120} (Y677F) were co-transfected, and SunTag-controlled SEAP secretion as well as RR120-controlled NanoLuc secretion were profiled in the culture supernatant after 24 h. Concentrations were 100 ng/mL RR120 and 0.02% (v/v) SunTag-containing bacterial lysate. NanoLuc concentrations are presented as RLU (relative luminescent units). Graph shows the mean±s.d. as a bar diagram overlaid with a dot plot of individual data points of n=9 biologically independent samples. *P<0.05, ****P<0.0001; ns, not significant. Numbers above the bars indicate fold changes of reporter (SEAP or NanoLuc) in the supernatant after 24 h induction, calculated by dividing mean concentration in the presence of inducer by mean concentration in the absence of inducer.

FIG. 14 shows that RR120 increases endogenous IL-10 secretion upon activation of JAK/STAT-GEMS_{RR120} or MAPK-GEMS_{RR120} signaling in WEN1.3 cells. Graphs show the mean±s.d. as a bar diagram overlaid with a dot plot of individual data points of n=9 biologically independent samples. *P<0.05, ****P<0.0001; ns, not significant. Numbers above the bars indicate fold changes of IL-10 concentration in the supernatant after 24 h induction, calculated by dividing mean concentration in the presence of inducer by mean concentration in the absence of inducer.

FIG. 15 shows the viability of HEK-293T cells in the presence of caffeine. HEK-293T cells were exposed to increasing concentrations of caffeine in standard cell culture medium. After 24 hours, cellular viability was assessed with a CCK-8 assay. Data are shown as the mean in bar graphs and symbols indicate individual data points. The data displayed represent three independent experiments (n=3).

FIG. 16 shows a schematic of the caffeine-inducible protein dimerization system based on the camelid-derived single-domain antibody aCaffVHH. aCaffVHH homodimerizes in the presence of caffeine and can be used to reconstitute synthetic transcription factors or signaling cascades that fine-tune caffeine-responsive gene expression.

FIG. 17A shows the caffeine-sensing circuit based on the heterodimerization of aCaffVHH-TetR (pDB307) and aCaffVHH-VP_{min×4} (pDB335), leading to direct transcriptional activation. The caffeine dose-response relationship was quantified with the reporter gene SEAP (P_{tetO7}-SEAP-pA_{SV40}, pMF111). FIG. 17B shows the caffeine-sensing circuit based on the IL13 receptor and the JAK/STATE pathway. Caffeine-induced heterodimerization of aCaffVHH-IL13Rα1 (pDB323) and aCaffVHH-IL4Rα (pDB324) leads to phosphorylation of STATE (pLS16) by JAK kinases and subsequent transcriptional activation of the STATE-responsive promoter P_{STAT6}. The caffeine dose-response relationship was quantified with the reporter gene SEAP (P_{STAT6}-SEAP-pA_{SV40}, pLS12). FIG. 17C shows the caffeine-sensing circuit based on the MAPK pathway. Caffeine-induced homodimerization of mFGFR1₄₀₅₋₈₂₂-aCaffVHH (pDB395) led to phosphorylation of MEK1/2 and downstream signaling of the MAPK cascade. Rewiring the signaling cascade through the hybrid transcription factor TetR-Elk1 (MKp37) led to expression of the reporter gene SEAP (P_{tetO7}-SEAP-pA_{SV40}, pMF111), enabling quantification of the caffeine dose-response relationship. FIG. 17D shows the caffeine-sensing circuit based on the Epo receptor and the JAK/STAT3 pathway. Caffeine-induced homodimerization of aCaffVHH-EpoRₘ-IL-6RBₘ (pDB306) leads to phosphorylation of STAT3 by JAK kinases and subsequent transcriptional activation of the STAT3-responsive promoter P_{STAT3}. The caffeine dose-response relationship was quantified with the reporter gene SEAP (P_{STAT3}-SEAP-pA_{SV40}, pLS13). Data are shown as the mean in bar graphs and symbols indicate individual data points. The data displayed represent three independent experiments (n=3).

FIG. 18A shows the orthogonality of STAT3 signaling and TetR-dependent promoter. HEK-293T cells transfected with pDB306 (P_{hCMV}-aCaffVHH-EpoRₘ-IL-6RBₘ-pAbGH) and pMF111 (P_{tetO7}-SEAP-pA_{SV40}) were exposed to increasing concentrations of caffeine in standard cell culture medium 16 hours after transfection. SEAP was measured 24 hours after the addition of caffeine in the supernatant of the cells. FIG. 18B shows the nonlinear response with the combination of caffeine-sensing systems. HEK-293T cells transfected with pDB306 (P_{hCMV}-aCaffVHH-EpoRₘ-IL-6RBₘ-pAbGH), pLS13 (P_{STAT3}-SEAP-pA_{SV40}), pDB307 (P_{SV40}-TetR-aCaffVHH-pA_{SV40}), pDB335 (P_{CAG}-aCaffVHH-VP_{min×4}-pA_{βG}) and pMF111 (P_{tetO7}-SEAP-pA_{SV40}) were exposed to increasing concentrations of caffeine in standard cell culture medium 16 hours after transfection. SEAP was measured 24 hours after the addition of caffeine in the supernatant of the cells. Data are shown as the mean in bar graphs and symbols indicate individual data points. The data displayed represent three independent experiments (n=3).

FIG. 19A shows the functionality of C-STAR in hMSC-hTERT cells. hMSC-hTERT cells were transiently transfected with pDB306 (P_{hCMV}-aCaffVHH-EpoRₘ-IL-6RBₘ-pA_{bGH}) and pLS13 (P_{STAT3}-SEAP-pA_{SV40}). Sixteen hours after transfection, the cells were exposed to increasing concentrations of caffeine in standard cell culture medium. The caffeine dose-response relationship was quantified in terms of SEAP expression after 24 h. FIG. 19B shows the caffeine-responsiveness of polyclonal C-STAR_{DB1} cells. Polyclonal C-STAR_{DB1} cells were exposed to increasing caffeine concentrations to examine their sensitivity. Supernatant levels of SEAP were quantified after 24 h. Data are shown as the mean in bar graphs and symbols indicate individual data points. The data displayed represent three independent experiments (n=3).

FIG. 20A shows the caffeine sensitivity of monoclonal C-STAR_{DB2} cell line. FIG. 20B shows the caffeine sensitivity of monoclonal C-STAR_{DB3} cell line. FIG. 20C shows the caffeine sensitivity of monoclonal C-STAR_{DB4} cell line. FIG. 20D shows the caffeine sensitivity of monoclonal C-STAR_{DB5} cell line. C-STAR_{DB2}, C-STAR_{DB3}, C-STAR_{DB4} and C-STAR_{DB5} cells transfected with the reporter plasmid pLS13 (P_{STAT3}-SEAP-pA_{SV40}) were exposed to increasing concentrations of caffeine in standard cell culture medium at 16 hours after transfection. SEAP was measured 24 hours after the addition of caffeine in the supernatant of the cells. Data are shown as the mean in bar graphs and symbols indicate individual data points. The data displayed represent three independent experiments (n=3).

FIG. 21 shows the caffeine exposure time needed for the activation of the C-STAR system. C-STAR_{DB1} cells were exposed to H₂O or 10 μM caffeine in standard cell culture medium for different periods of time to determine the minimum exposure time needed for induction. After the indicated time, the caffeinated medium was replaced with standard cell culture medium and SEAP expression proceeded for 24 h before quantification. Data are shown as the mean in bar graphs and symbols indicate individual data points. The data displayed represent three independent experiments (n=3).

FIG. 22 shows the specificity of C-STAR_{DB1} for caffeine versus several analogs. C-STAR_{DB1} cells transfected with the reporter plasmid pLS13 (P_{STAT3}-SEAP-pA_{SV40}) were exposed to increasing concentrations of caffeine, theophylline, theobromine and paraxanthine in standard cell culture medium at 16 hours after transfection. SEAP was measured 24 hours after the addition of caffeine in the supernatant of the cells. Data are shown as the mean in bar graphs and symbols indicate individual data points. The data displayed represent three independent experiments (n=3).

FIG. 23A shows the response time of the C-STAR system to caffeine. C-STAR_{DB1} cells were exposed to H₂O or increasing concentrations of caffeine in standard cell culture medium to determine the response time of the system. Supernatant samples containing SEAP were taken every 12 h for 72 h. The data displayed represent the means±s.d. of three independent experiments (n=3). FIG. 23B shows reversibility of the C-STAR system. C-STAR_{DB1} cells were alternately exposed to H₂O and 10 μM caffeine in standard cell culture medium to show the reversibility of the system. Supernatant samples containing SEAP were taken every three hours for nine hours per day. The data displayed represent the means±s.d. of three independent experiments (n=3).

FIG. 24 shows an illustration of the tested solutions with their respective caffeine concentration. From left to right, the boxes correspond to Nesquik® capsules, Forest Fruits® (herbal tea), Vivalto Lungo Decaffeinato®, Volluto Decaffeinato®, Decaffeinato Intenso®, Arpeggio Decaffeinato®, CocaCola®, Mediterranean® (green tea), Marrakech® (green tea), Earl Grey® (black tea), Starbucks® Coffee Frappuccino, Starbucks® Caramel Macchiato, Red Bull®, Bukeela ka Ethiopia®, Vivalto Lungo®, Starbucks® Coffee, Capriccio®, Livanto®, Apfelstrudel®, Volluto®, Roma®, Arpeggio®, Ristretto®, Dharkan®, Military Energy Gum®, and Kazaar®. The indicated caffeine concentrations were calculated from the specifications of the vendor regarding the amount of caffeine in each beverage.

FIG. 25A shows the quantification of the caffeine concentration in coffee from Nespresso Grand Cru® capsules. FIG. 25B shows the quantification of the caffeine concentration in coffee from other commercially available caffeine sources. Caffeine-containing samples were added to C-STAR_{DB1} cells with a dilution of 1:50,000. A standard curve obtained with pure caffeine enabled conversion of the quantified SEAP levels to caffeine concentrations in the original samples. Each beverage was prepared or bought on three separate occasions and the data represent the quantification of each replicate in triplicate (n=3). Data are shown as the mean in bar graphs and symbols indicate individual data points. The caffeine concentration indicated by the vendor is shown on the right.

FIG. 26 shows the functionality of encapsulated C-STAR_{DB1} cells in the presence of caffeine. C-STAR_{DB1} cells transfected with the reporter plasmid pLS13 (P_{STAT3}-SEAP-pA_{SV40}) and encapsulated in vascularized microcontainers were exposed to increasing concentrations of caffeine in standard cell culture medium. After 24 hours, SEAP activity was quantified in the supernatant of the cells. Data are shown as the mean in bar graphs and symbols indicate individual data points. The data displayed represent three independent experiments (n=3).

FIG. 27A shows the impact of microcapsule implants on the TNF-α level. FIG. 27B shows the impact of microcapsule implants on the IL-6 level. Wild-type mice were intraperitoneally implanted with microencapsulated C-STAR_{DB6} cells and blood samples were collected at 12 hours before (control) and at 12, 36 and 60 hours after implantation. TNF-α and IL-6 levels in the bloodstream of mice were determined by ELISA. The data displayed are mean±SEM (n=8 mice). n.s. not significant (Welch's t test).

FIG. 28A shows the SEAP level in in the bloodstream of mice quantified at 24 h after coffee intake. FIG. 28B shows the SEAP level in in the bloodstream of mice quantified at 48 h after coffee intake. FIG. 28C shows the SEAP level in in the bloodstream of mice quantified at 24 h after coffee intake five days after implantation. FIG. 28D shows the SEAP level in in the bloodstream of mice quantified at 48 h after coffee intake five days after implantation. Wild-type mice were intraperitoneally implanted with microencapsulated C-STAR_{DB1} (Caffeine Receptor: +) or control HEK-293T cells only transfected with pLS13 (Caffeine Receptor: −; P_{STAT3}-SEAP-pA_{SV40}), and stimulated by oral administration of 300 μL Volluto® coffee (Coffee: +) or H₂O (Coffee: −). SEAP levels in the bloodstream of mice were quantified at 24 h and 48 h after coffee intake. Five days after implantation, the same mice received another oral administration of 300 μL Volluto® coffee (Coffee: +) or H₂O (Coffee: −). SEAP levels in the bloodstream were quantified at 24 h and 48 h after the second coffee intake. The data displayed are mean±SEM (n=8). ***P<0.001 versus control, n.s. not significant (Welch's t test).

FIG. 29A shows the assessment of the caffeine-induced expression of shGLP-1. C-STAR_{DB1} or C-STAR_{DB3} cells stably expressing the caffeine receptor (P_{hEF-1α}-aCaffVHH-EpoRₘ-IL-6RBₘ-pA_{SV40}, pDB326) were transiently transfected with pDB387 (P_{STAT3}-shGLP-1-pA_{SV40}) and exposed to H₂O or caffeine 16 hours after transfection. After 48 hours, shGLP-1 expression was quantified with a Mouse IgG ELISA Kit (ICL Lab) in the supernatant of the cells. The data displayed represent four independent experiments (n=4). FIG. 29B shows the validation of the polyclonal, shGLP-1 expressing C-STAR_{DB6} cell line. C-STAR_{DB6} cells stably expressing both the caffeine receptor (P_{hEF-1α}-aCaffVHH-EpoRₘ-IL-6RBₘ-pA_{SV40}, pDB326) as well as shGLP1 (P_{STAT3}-shGLP-1-pA_{SV40}, pDB387) were exposed to H₂O or caffeine. After 48 hours, shGLP-1 expression was quantified with a Mouse IgG ELISA Kit (ICL Lab) in the supernatant of the cells. Data are shown as the mean in bar graphs and symbols indicate individual data points. The data displayed represent four independent experiments (n=4).

FIG. 30A shows the pharmacokinetics of caffeine in vivo. FIG. 30B shows the pharmacokinetics of shGLP-1 in vivo. Wild-type mice were intraperitoneally implanted with microencapsulated C-STAR_{DB6} cells and received a single oral administration of 300 μL Nespresso Volluto® coffee. Caffeine and shGLP-1 levels in the bloodstream of mice were recorded every six hours for 60 hours. The data displayed are mean±SEM (n=8). ****P<0.0001 versus control (Welch's t test).

FIG. 31A shows the dose-dependent production of shGLP-1 in the bloodstream of mice. FIG. 31B shows the fast glycemia of mice. Wild-type mice were intraperitoneally implanted with microencapsulated C-STAR_{DB6} cells, and received single oral administrations of different Nespresso® coffee formulations having different caffeine concentrations (ingestion volume: 300 μL). Dose-dependent production of shGLP-1 in the bloodstream of mice was recorded 24 h after coffee intake. Data are shown as the mean in bar graphs and symbols indicate individual mice (n=8). Fasting glycemia was recorded for 72 h. The range of homeostatic fasting glycemia is indicated with a gray box. The data displayed are mean±SEM (n=8 mice). Welch's t test showed no significant differences between groups.

FIGS. 32A, 32B, 32C, and 32D show the caffeine-dependent insulinotropic action of shGLP-1, with FIG. 32A showing the fasting glycemia (the range of homeostatic fasting glycemia is indicated with a gray box), FIG. 32B showing the blood active GLP-1, FIG. 32C showing the 4 h postprandial insulin level, and FIG. 32D showing the results of intraperitoneal glucose tolerance tests. Wild type (WT) or diet-induced obese mice (DIO) were intraperitoneally implanted with microencapsulated C-STAR_{DB6} cells or control HEK-293T cells containing only pDB387 (P_{STAT3}-shGLP-1-pA_{SV40}) and received daily oral doses of 300 μL Nespresso Volluto® coffee. Fasting glycemia, blood active GLP-1, and 4 h postprandial insulin levels were recorded for 14 days. Intraperitoneal glucose tolerance tests were performed by administration of 2 g kg⁻¹ aqueous D-glucose. All data displayed are mean±SEM (n=10 mice). Comparisons were made with Welch's t test: *P<0.05, **P<0.01, ***P<0.001, ****P<0.0001 vs. control, n.s. not significant.

FIG. 33A shows the caffeine-dependent cardiovascular effects. Heart rate of the same mice shown in FIGS. 32A-D was measured prior to the collection of blood samples. FIG. 33B shows the caffeine-triggered shGLP-1-mediated effects on body weight. On day 15, the body weights of individual mice shown in FIGS. 32A-D and FIG. 33A were compared to their initial body weights (day 1; prior to first coffee intake). The confidence interval of the balance is indicated by a gray box. All data displayed are mean±SEM (n=10 mice). Comparisons were made with Welch's t test: *P<0.05, **P<0.01, ***P<0.001, ****P<0.0001 vs. control, n.s. not significant.

FIGS. 34A, 34B, and 34C show the caffeine-dependent insulinotropic action of shGLP-1, with FIG. 34A showing the blood active GLP-1, FIG. 34B showing the 4 h postprandial insulin level, and FIG. 34C showing the results of intraperitoneal glucose tolerance tests. FIG. 34D shows the caffeine-dependent cardiovascular effects. Wild type (WT) or leptin receptor-deficient mice (db/db) were intraperitoneally implanted with different doses of microencapsulated C-STAR_{DB6} cells (0 to 1×10⁷ cells) or 1×10⁷ control HEK-293T cells containing only pDB387 (P_{STAT3}-shGLP-1-pA_{SV40}), and received an oral dose of 300 μL Nespresso Volluto® coffee. Blood active GLP-1 and 4 h postprandial insulin levels were recorded before cell implantation and 1 day afterwards. Intraperitoneal glucose tolerance tests were performed by administration of 2 g kg⁻¹ aqueous D-glucose. Heart rate of the same mice was measured prior to the collection of blood samples. All data displayed are mean±SEM (n=10 mice). Comparisons were made with Welch's t test: *P<0.05, **P<0.01, ***P<0.001, ****P<0.0001 vs. control, n.s. not significant.

### DETAILED DESCRIPTION

### Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, N.Y. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s) ±one standard deviation of that value(s).

The term “Erythropoietin Receptor (EpoR)” refers a member of the cytokine receptor family. It is encoded by the EPOR gene in humans. EpoR pre-exists as dimers which upon binding of a 30 kDa ligand erythropoietin (Epo), changes its homodimerized conformation. These conformational changes result in the autophosphorylation of Jak2 kinases that are pre-associated with the receptor (i.e., EpoR does not possess intrinsic kinase activity and depends on Jak2 activity). One well-established function of EpoR is to promote proliferation and rescue of erythroid (red blood cell) progenitors from apoptosis. An exemplary RefSeq accession number for human EpoR precursor is NP 000112.1 as shown on the NCBI website as of Oct. 1, 2018. An exemplary RefSeq accession number for mouse EpoR precursor is NP_034279.3 as shown on the NCBI website as of Oct. 1, 2018. Exemplary amino acid sequences for mouse EpoR precursor, mature EpoR, and EpoR isoform EpoR-S and human EpoR precursor, mature EpoR, and EpoR isoforms EpoR-S and EpoR-T are shown below.

| Name | Amino Acid Sequence |
|---|---|
| EpoR | MDKLRVPLWPRVGPLCLLLAGAAWAPSPSLPDPKFESKAALLASRGSE |
| precursor | ELLCFTQRLEDLVCFWEEAASSGMDFNYSFSYQLEGESRKSCSLHQAPT |
| (mouse) | VRGSVRFWCSLPTADTSSFVPLELQVTEASGSPRYHRIIHINEVVLLDAP |
| | AGLLARRAEEGSHVVLRWLPPPGAPMTTHIRYEVDVSAGNRAGGTQR |
| | VEVLEGRTECVLSNLRGGTRYTFAVRARMAEPSFSGFWSAWSEPASLL |
| | TASDLDPLILTLSLILVLISLLLTVLALLSHRRTLQQKIWPGIPSPESEFEG |
| | LFTTHKGNFQLWLLQRDGCLWWSPGSSFPEDPPAHLEVLSEPRWAVTQ |
| | AGDPGADDEGPLLEPVGSEHAQDTYLVLDKWLLPRTPCSENLSGPGGS |
| | VDPVTMDEASETSSCPSDLASKPRPEGTSPSSFEYTILDPSSQLLCPRALP |
| | PELPPTPPHLKYLYLVVSDSGISTDYSSGGSQGVHGDSSDGPYSHPYENS |
| | LVPDSEPLHPGYVACS (SEQ ID NO: 1) |
| Mature | APSPSLPDPKFESKAALLASRGSEELLCFTQRLEDLVCFWEEAASSGMD |
| EpoR | FNYSFSYQLEGESRKSCSLHQAPTVRGSVRFWCSLPTADTSSFVPLELQ |
| (mouse) | VTEASGSPRYHRIIHINEVVLLDAPAGLLARRAEEGSHVVLRWLPPPGA |
| | PMTTHIRYEVDVSAGNRAGGTQRVEVLEGRTECVLSNLRGGTRYTFAV |
| | RARMAEPSFSGFWSAWSEPASLLTASDLDPLILTLSLILVLISLLLTVLAL |
| | LSHRRTLQQKIWPGIPSPESEFEGLFTTHKGNFQLWLLQRDGCLWWSPG |
| | SSFPEDPPAHLEVLSEPRWAVTQAGDPGADDEGPLLEPVGSEHAQDTY |
| | LVLDKWLLPRTPCSENLSGPGGSVDPVTMDEASETSSCPSDLASKPRPE |
| | GTSPSSFEYTILDPSSQLLCPRALPPELPPTPPHLKYLYLVVSDSGISTDYS |
| | SGGSQGVHGDSSDGPYSHPYENSLVPDSEPLHPGYVACS |
| | (SEQ ID NO: 2) |
| Isoform | MDKLRVPLWPRVGPLCLLLAGAAWAPSPSLPDPKFESKAALLASRGSE |
| EpoR-S; | ELLCFTQRLEDLVCFWEEAASSGMDFNYSFSYQLEGESRKSCSLHQAPT |
| soluble | VRGSVRFWCSLPTADTSSFVPLELQVTEASGSPRYHRIIHINEVVLLDAP |
| form | AGLLARRAEEGSHVVLRWLPPPGAPMTTHIRYEVDVSAGNRAGGTQR |
| (mouse) | VEVLEGRTECVLSNLRGGTRYTFAVRARMAEPSFSGFWSAWSEPASLL |
| | TASGEALVPRGAGGAGPNTRQTP (SEQ ID NO: 3) |
| EpoR | MDHLGASLWPQVGSLCLLLAGAAWAPPPNLPDPKFESKAALLAARGP |
| precursor | EELLCFTERLEDLVCFWEEAASAGVGPGNYSFSYQLEDEPWKLCRLHQ |
| (human) | APTARGAVRFWCSLPTADTSSFVPLELRVTAASGAPRYHRVIHINEVVL |
| | LDAPVGLVARLADESGHVVLRWLPPPETPMTSHIRYEVDVSAGNGAGS |
| | VQRVEILEGRTECVLSNLRGRTRYTFAVRARMAEPSFGGFWSAWSEPV |
| | SLLTPSDLDPLILTLSLILVVILVLLTVLALLSHRRALKQKIWPGIPSPESE |
| | FEGLFTTHKGNFQLWLYQNDGCLWWSPCTPFTEDPPASLEVLSERCWG |
| | TMQAVEPGTDDEGPLLEPVGSEHAQDTYLVLDKWLLPRNPPSEDLPGP |
| | GGSVDIVAMDEGSEASSCSSALASKPSPEGASAASFEYTILDPSSQLLRP |
| | WTLCPELPPTPPHLKYLYLVVSDSGISTDYSSGDSQGAQGGLSDGPYSN |
| | PYENSLIPAAEPLPPSYVACS (SEQ ID NO: 4) |
| Mature | APPPNLPDPKFESKAALLAARGPEELLCFTERLEDLVCFWEEAASAGVG |
| EpoR | PGNYSFSYQLEDEPWKLCRLHQAPTARGAVRFWCSLPTADTSSFVPLEL |
| (human) | RVTAASGAPRYHRVIHINEVVLLDAPVGLVARLADESGHVVLRWLPPP |
| | ETPMTSHIRYEVDVSAGNGAGSVQRVEILEGRTECVLSNLRGRTRYTFA |
| | VRARMAEPSFGGFWSAWSEPVSLLTPSDLDPLILTLSLILVVILVLLTVL |
| | ALLSHRRALKQKIWPGIPSPESEFEGLFTTHKGNFQLWLYQNDGCLWW |
| | SPCTPFTEDPPASLEVLSERCWGTMQAVEPGTDDEGPLLEPVGSEHAQD |
| | TYLVLDKWLLPRNPPSEDLPGPGGSVDIVAMDEGSEASSCSSALASKPS |
| | PEGASAASFEYTILDPSSQLLRPWTLCPELPPTPPHLKYLYLVVSDSGIST |
| | DYSSGDSQGAQGGLSDGPYSNPYENSLIPAAEPLPPSYVACS |
| | (SEQ ID NO: 5) |
| Isoform | MDHLGASLWPQVGSLCLLLAGAAWAPPPNLPDPKFESKAALLAARGP |
| EpoR-S; | EELLCFTERLEDLVCFWEEAASAGVGPGNYSFSYQLEDEPWKLCRLHQ |
| soluble | APTARGAVRFWCSLPTADTSSFVPLELRVTAASGAPRYHRVIHINEVVL |
| form | LDAPVGLVARLADESGHVVLRWLPPPETPMTSHIRYEVDVSAGNGAGS |
| (human) | VQRGTVFLSPDWLSSTRARPHVIYFCLLRVPRPDSAPRWRSWRAAPSV |
| | C (SEQ ID NO: 6) |
| Isoform | MDHLGASLWPQVGSLCLLLAGAAWAPPPNLPDPKFESKAALLAARGP |
| EpoR-T; | EELLCFTERLEDLVCFWEEAASAGVGPGNYSFSYQLEDEPWKLCRLHQ |
| truncated | APTARGAVRFWCSLPTADTSSFVPLELRVTAASGAPRYHRVIHINEVVL |
| form | LDAPVGLVARLADESGHVVLRWLPPPETPMTSHIRYEVDVSAGNGAGS |
| (human) | VQRVEILEGRTECVLSNLRGRTRYTFAVRARMAEPSFGGFWSAWSEPV |
| | SLLTPSDLDPLILTLSLILVVILVLLTVLALLSHRRALKQKIWPGIPSPESE |
| | FEGLFTTHKGNFQVGGLVVPSVPGLPCFLQPNCRPL (SEQ ID NO: 7) |

The term “chimeric ligand receptor” as used herein refers to a ligand receptor that comprises domains derived from multiple distinct protein sequences. Chimeric ligand receptors of the present disclosure do not include natural receptors or wild-type receptors, such as a wild-type cytokine receptor or a wild-type erythropoietin receptor (EpoR). A chimeric ligand receptor of the present disclosure comprises one or more receptor subunits. Each of the receptor subunits comprises a scaffold domain, a ligand binding domain, and an intracellular signaling domain.

The term “scaffold domain” as used herein refers to at least a portion of an extracellular domain and transmembrane domain of a receptor that is activated upon ligand binding. The binding of ligand can lead to conformational change(s) or conformation reorganization of the scaffold domain, which modulates activity of an intracellular signaling domain that is operably linked to the scaffold domain. The scaffold domain can be derived from the extracellular domain and transmembrane domain of a cytokine receptor, such as EpoR. In some embodiments, the scaffold domain may be inert to binding of its native ligand. For example, if the scaffold domain is derived from the extracellular domain and transmembrane domain of EpoR, it is inert to erythropoietin binding.

The term “ligand binding domain” as used herein refers to the domain of a chimeric ligand binding receptor of the present disclosure that is operably linked to the extracellular domain of the scaffold domain. Ligand binding domains of the present disclosure are chimeric in that they are not derived from the same parental protein as the scaffold domain and do not bind to the native ligand of the receptor that the scaffold domain is derived from. For example, if the scaffold domain is derived from EpoR, the ligand binding domain does not bind to the native ligand of EpoR, erythropoietin.

The term “intracellular signaling domain” as used herein refers to the domain of the chimeric ligand binding receptor that is operably linked to the transmembrane domain of the scaffold domain. Intracellular signaling domains of the present disclosure are chimeric in that they are not derived from the same parental protein as the scaffold domain and are not activated by the native ligand of the parental receptor that the scaffold domain is derived from. For example, if the scaffold domain is derived from EpoR, the intracellular signaling domain is not activated by the native ligand of EpoR, erythropoietin.

The term “native ligand” as used herein refers to a wild-type ligand that naturally binds to the parental receptor from which the scaffold domain is derived. For example, if the scaffold domain is derived from EpoR, the native ligand is erythropoietin. The scaffold domain is inert (i.e., unresponsive) to its native ligand. The scaffold domain can contain modifications that render it unresponsive to its native ligand.

### Chimeric Ligand Receptor

In some aspects, disclosed herein is a chimeric ligand receptor.

In some embodiments, the chimeric ligand receptor comprises one receptor subunit. In some embodiments, the chimeric ligand receptor comprises more than one receptor subunits, such as two receptor subunits, three receptor subunits, four receptor subunits, five receptor subunits, six receptor subunits, or more than six receptor subunits.

In some embodiments, the chimeric ligand receptor subunit comprises a scaffold domain, a ligand binding domain, and an intracellular signaling domain.

In some embodiments, the two or more receptor subunits multimerize via the scaffold domain. In some embodiments, the multimerized receptor subunits comprise a dimer, a trimer, tetramer, pentamer, or hexamer. In some embodiments, the multimerized receptor subunits comprise a dimer.

In some embodiments, the multimerization of the receptor subunits occurs prior to ligand binding. In some embodiments, the multimerized receptor subunits are locked by transmembrane helix interactions in a conformation that prevents downstream signaling in the absence of ligand binding. In some embodiments, the ligand binding leads to a conformational reorganization. In some embodiments, the conformational reorganization may comprise a rotation of each scaffold domain around its own axis. In some embodiments, the conformational reorganization activates the intracellular signaling domains of each receptor subunit. In some embodiments, the conformational reorganization inhibits the intracellular signaling domains of each receptor subunit.

### Scaffold Domain

In some embodiments, the scaffold domain comprises an extracellular domain and a transmembrane domain. In some embodiments, the extracellular domain is operably linked to a ligand binding domain and the transmembrane domain is operably linked to an intracellular signaling domain.

In some embodiments, the scaffold domain comprises the extracellular domain and transmembrane domain of a receptor. In some embodiments, the scaffold domain comprises the extracellular domain and transmembrane domain of a transmembrane receptor, such as a cytokine receptor, having a preformed, inactive, dimeric structure on the cell surface that is activated upon ligand binding by a conformational reorganization (e.g., rotation) of the transmembrane domain (See, e.g., Maruyama I N, Bioessays, 2015, 37:959-967). In some embodiments, the scaffold domain comprises the extracellular domain and transmembrane domain of an erythropoietin receptor (EpoR).

In some embodiments, the scaffold domain is inert to its native ligand. In some embodiments, when the scaffold domain comprises the extracellular domain and transmembrane domain of an erythropoietin receptor (EpoR), the scaffold domain is inert to erythropoietin.

In some embodiments, the scaffold domain comprises one or more modifications. In some embodiments, the extracellular domain of the scaffold domain comprises one or more modifications. In some embodiments, the transmembrane domain of the scaffold domain comprises one or more modifications. In some embodiments, both the extracellular domain and the transmembrane domain of the scaffold domain comprise one or more modifications. In some embodiments, the modification comprises an amino acid insertion, an amino acid deletion, or an amino acid substitution. In some embodiments, the modification comprises a chemical modification, such as but not limited to acetylation, amidation, biotinylation, cysteinylation, deamidation, farnesylation, formylation, geranylgeranylation, glutathionylation, glycation, glycosylation, hydroxylation, methylation, mono-ADP-ribosylation, myristoylation, oxidation, palmitoylation, phosphorylation, poly(ADP-ribosyl)ation, stearoylation, or sulfation. In some embodiments, the modification renders the scaffold domain inert to its native ligand. In some embodiments, when the scaffold domain comprises the extracellular domain and transmembrane domain of an erythropoietin receptor (EpoR), the extracellular domain comprises an F93A amino acid substitution. In some embodiments, one or more additional amino acid residues are inserted adjacent to the transmembrane domain. In some embodiments, one or more additional amino acid residues are inserted within the transmembrane domain. In some embodiments, the one or more additional amino acid residues are alanine residues. In some embodiment, one or more positively charged amino acid residues are inserted C-terminal to the transmembrane domain. In some embodiments, the transmembrane domain further comprises one, two, three, or four additional alanine residues. In some embodiments, the one or more additional amino acid residues are inserted C-terminal to the transmembrane domain.

In some embodiments, the scaffold domain comprises an extracellular domain and transmembrane domain comprising an amino acid sequence having 60-100% sequence identity to SEQ ID NO: 8, such as 70-100%, 80-100%, 85-100%, 90-100%, 95-100%, 97-100%, or 99-100% sequence identity to APSPSLPDPKFESKAALLASRGSEELLCFTQRLEDLVCFWEEAASSGMDFNYSFSYQLEG ESRKSCSLHQAPTVRGSVRFWCSLPTADTSSFVPLELQVTEASGSPRYHRIIHINEVVLLD APAGLLARRAEEGSHVVLRWLPPPGAPMTTHIRYEVDVSAGNRAGGTQRVEVLEGRTE CVLSNLRGGTRYTFAVRARMAEPSFSGFWSAWSEPASLLTASDLDPLILTLSLILVLISLL LTVLALLS (SEQ ID NO: 8). In some embodiments, the scaffold domain comprises an extracellular domain and transmembrane domain comprising an amino acid sequence having 60% or greater sequence identity to SEQ ID NO: 8. In some embodiments, the scaffold domain comprises an extracellular domain and transmembrane domain comprising an amino acid sequence having 70% or greater sequence identity to SEQ ID NO: 8. In some embodiments, the scaffold domain comprises an extracellular domain and transmembrane domain comprising an amino acid sequence having 80% or greater sequence identity to SEQ ID NO: 8. In some embodiments, the scaffold domain comprises an extracellular domain and transmembrane domain comprising an amino acid sequence having 85% or greater sequence identity to SEQ ID NO: 8. In some embodiments, the scaffold domain comprises an extracellular domain and transmembrane domain comprising an amino acid sequence having 90% or greater sequence identity to SEQ ID NO: 8. In some embodiments, the scaffold domain comprises an extracellular domain and transmembrane domain comprising an amino acid sequence having 95% or greater sequence identity to SEQ ID NO: 8. In some embodiments, the scaffold domain comprises an extracellular domain and transmembrane domain comprising an amino acid sequence having 97% or greater sequence identity to SEQ ID NO: 8. In some embodiments, the scaffold domain comprises an extracellular domain and transmembrane domain comprising an amino acid sequence having 99% or greater sequence identity to SEQ ID NO: 8. In some embodiments, the scaffold domain comprises an extracellular domain and transmembrane domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 8. In some embodiments, the scaffold domain comprises an extracellular domain and transmembrane domain comprising an amino acid sequence having 100% sequence identity to SEQ ID NO: 8.

In some embodiments, the extracellular domain comprises an amino acid sequence having 60-100% sequence identity to SEQ ID NO: 9, such as 70-100%, 80-100%, 85-100%, 90-100%, 95-100%, 97-100%, or 99-100% sequence identity to APSPSLPDPKFESKAALLASRGSEELLCFTQRLEDLVCFWEEAASSGMDFNYSFSYQLEG ESRKSCSLHQAPTVRGSVRFWCSLPTADTSSFVPLELQVTEASGSPRYHRIIHINEVVLLD APAGLLARRAEEGSHVVLRWLPPPGAPMTTHIRYEVDVSAGNRAGGTQRVEVLEGRTE CVLSNLRGGTRYTFAVRARMAEPSFSGFWSAWSEPASLLTASDLDP (SEQ ID NO: 9). In some embodiments, the extracellular domain comprises an amino acid sequence having 60% or greater sequence identity to SEQ ID NO: 9. In some embodiments, the extracellular domain comprises an amino acid sequence having 70% or greater sequence identity to SEQ ID NO: 9. In some embodiments, the extracellular domain comprises an amino acid sequence having 80% or greater sequence identity to SEQ ID NO: 9. In some embodiments, the extracellular domain comprises an amino acid sequence having 85% or greater sequence identity to SEQ ID NO: 9. In some embodiments, the extracellular domain comprises an amino acid sequence having 90% or greater sequence identity to SEQ ID NO: 9. In some embodiments, the extracellular domain comprises an amino acid sequence having 95% or greater sequence identity to SEQ ID NO: 9. In some embodiments, the extracellular domain comprises an amino acid sequence having 97% or greater sequence identity to SEQ ID NO: 9. In some embodiments, the extracellular domain comprises an amino acid sequence having 99% or greater sequence identity to SEQ ID NO: 9. In some embodiments, the extracellular domain comprises an amino acid sequence having 85%, 86%, 87%, 88%, 89%, 90%, 91%, 92%, 93%, 94%, 95%, 96%, 97%, 98%, 99%, or 100% sequence identity to SEQ ID NO: 9. In some embodiments, the extracellular domain comprises an amino acid sequence having 100% sequence identity to SEQ ID NO: 9.

In some embodiments, the transmembrane domain comprises an amino acid sequence having 60-100% sequence identity to SEQ ID NO: 10, such as 70-100%, 80-100%, 85-100%, 90-100%, 95-100%, 97-100%, or 99-100% sequence identity to LILTLSLILVLISLLLTVLALLS (SEQ ID NO: 10). In some embodiments, the transmembrane domain comprises an amino acid sequence having 60% or greater sequence identity to SEQ ID NO: 10. In some embodiments, the transmembrane domain comprises an amino acid sequence having 70% or greater sequence identity to SEQ ID NO: 10. In some embodiments, the transmembrane domain comprises an amino acid sequence having 80% or greater sequence identity to SEQ ID NO: 10. In some embodiments, the transmembrane domain comprises an amino acid sequence having 85% or greater sequence identity to SEQ ID NO: 10. In some embodiments, the transmembrane domain comprises an amino acid sequence having 90% or greater sequence identity to SEQ ID NO: 10. In some embodiments, the transmembrane domain comprises an amino acid sequence having 95% or greater sequence identity to SEQ ID NO: 10. In some embodiments, the transmembrane domain comprises an amino acid sequence having 97% or greater sequence identity to SEQ ID NO: 10. In some embodiments, the transmembrane domain comprises an amino acid sequence having 99% or greater sequence identity to SEQ ID NO: 10. In some embodiments, the transmembrane domain comprises an amino acid sequence having 85%, 86%, 87%, 88%, 89%, 90%, 91%, 92%, 93%, 94%, 95%, 96%, 97%, 98%, 99%, or 100% sequence identity to SEQ ID NO: 10. In some embodiments, the transmembrane domain comprises an amino acid sequence having 100% sequence identity to SEQ ID NO: 10.

In some embodiments, the scaffold domain comprises an extracellular domain comprising an amino acid sequence having 80-100% sequence identity to SEQ ID NO: 9, such as 85-100%, 90-100%, 95-100%, 97-100%, or 99-100% sequence identity to SEQ ID NO: 9 and a transmembrane domain comprising an amino acid sequence having 80-100% sequence identity to SEQ ID NO: 10 such as 85-100%, 90-100%, 95-100%, 97-100%, or 99-100% sequence identity to SEQ ID NO: 10. In some embodiments, the scaffold domain comprises an extracellular domain comprising an amino acid sequence having 80% or greater sequence identity to SEQ ID NO: 9 and a transmembrane domain comprising an amino acid sequence having 80% or greater sequence identity to SEQ ID NO: 10. In some embodiments, the scaffold domain comprises an extracellular domain comprising an amino acid sequence having 85% or greater sequence identity to SEQ ID NO: 9 and a transmembrane domain comprising an amino acid sequence having 85% or greater sequence identity to SEQ ID NO: 10. In some embodiments, the scaffold domain comprises an extracellular domain comprising an amino acid sequence having 90% or greater sequence identity to SEQ ID NO: 9 and a transmembrane domain comprising an amino acid sequence having 90% or greater sequence identity to SEQ ID NO: 10. In some embodiments, the scaffold domain comprises an extracellular domain comprising an amino acid sequence having 95% or greater sequence identity to SEQ ID NO: 9 and a transmembrane domain comprising an amino acid sequence having 95% or greater sequence identity to SEQ ID NO: 10. In some embodiments, the scaffold domain comprises an extracellular domain comprising an amino acid sequence having 97% or greater sequence identity to SEQ ID NO: 9 and a transmembrane domain comprising an amino acid sequence having 97% or greater sequence identity to SEQ ID NO: 10. In some embodiments, the scaffold domain comprises an extracellular domain comprising an amino acid sequence having 99% or greater sequence identity to SEQ ID NO: 9 and a transmembrane domain comprising an amino acid sequence having 99% or greater sequence identity to SEQ ID NO: 10. In some embodiments, the scaffold domain comprises an extracellular domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 9 and a transmembrane domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 10. In some embodiments, the scaffold domain comprises an extracellular domain comprising an amino acid sequence having 100% sequence identity to SEQ ID NO: 9 and a transmembrane domain comprising an amino acid sequence having 100% sequence identity to SEQ ID NO: 10.

### Ligand Binding Domain

In some embodiments, the ligand binding domain does not bind a native ligand of the scaffold domain. In some embodiments, the ligand binding domain does not comprise an endogenous ligand binding domain of the scaffold domain. In some embodiments, when the scaffold domain is derived from a cytokine receptor, the ligand binding domain does not bind to the cognate cytokine. In some embodiments, when the scaffold domain is derived from a cytokine receptor, the ligand binding domain is not derived from the same cytokine receptor. In some embodiments, when the scaffold domain is derived from an erythropoietin receptor (EpoR), the ligand binding domain does not bind to erythropoietin. In some embodiments, when the scaffold domain is derived from an erythropoietin receptor (EpoR), the ligand binding domain is not derived from the erythropoietin receptor (EpoR).

In some embodiments, the ligand binding domain binds to a soluble ligand that is a synthetic or designer ligand. In some embodiments, the ligand binding domain binds to a soluble ligand that is a natural ligand. In some embodiments, the ligand binding domain binds to a soluble ligand selected from the group consisting of a protein complex, a protein, a peptide, a nucleic acid, a small molecule, and a chemical agent. In some embodiments, the soluble ligand is selected from the group consisting of an antigen, a cytokine, a survival factor, a chemokine, a hormone, a transmitter, a growth factor, extracellular matrix, and a death factor. In some embodiments, the ligand binding domain binds to caffeine. In some embodiments, the ligand binding domain binds to rapamycin. In some embodiments, the ligand binding domain binds to RR120. In some embodiments, the ligand binding domain binds to nicotine. In some embodiments, the ligand binding domain binds to an antigen. In some embodiments, the ligand binding domain binds to a cancer antigen. In some embodiments, the ligand binding domain binds to a tumor antigen. In some embodiments, the ligand binding domain binds to a pathogen antigen. In some embodiments, the ligand binding domain binds to a prostate-specific antigen (PSA). In some embodiments, the ligand binding domain binds to a peptide tag. In some embodiments, the ligand binding domain binds to a SunTag.

In some embodiments, the ligand binding domains of each receptor subunit are the same as one another. In some embodiments, the ligand binding domains of each receptor subunit are distinct from one another. In some embodiments, the ligand binding domain comprises an antibody, or antigen-binding fragment thereof. In some embodiments, the ligand binding domain comprises a single chain variable fragment (scFv), or a single-domain antibody (sdAb). In some embodiments, each of the ligand binding domains comprises a single chain variable fragment (scFv), optionally wherein each scFv specifically binds to a distinct epitope of the antigen. In some embodiments, the chimeric ligand receptor comprises two ligand binding domains, and wherein one ligand binding domain comprises an immunoglobulin heavy chain variable domain (V_{H}) and the second ligand binding domain comprises an immunoglobulin light chain variable domain (V_{L}). In some embodiments, the ligand binding domain comprises a single-domain VHH camelid antibody domain that homodimerizes in the presence of caffeine. In some embodiments, the ligand binding domain comprises a camelid heavy chain antibody domain (VHH) that homodimerizes in the presence of RR120. In some embodiments, the ligand binding domain comprises an immunoglobulin heavy chain variable domain (V_{H}) and an immunoglobulin light chain variable domain (V_{L}) of a nicotine antibody. In some embodiments, the ligand binding domain comprises a GCN4-specific scFv. In some embodiments, the ligand binding domain comprises an scFv that binds to the prostate-specific antigen (PSA). In some embodiments, the ligand binding domain comprises an FKBP-rapamycin binding protein (FRB) and an FK506 and rapamycin binding protein (FKBP). In some embodiments, the ligand binding domain comprises a leucine zipper domain. In some embodiments, the ligand binding domain comprises a PSD95-Dlg 1-zo-1 (PDZ) domain, a streptavidin domain and a streptavidin binding protein (SBP) domain, or a PYL domain and an ABI domain. In some embodiments, the ligand binding domain comprises a cyclophilin-Fas fusion protein (CyP-Fas) and a FK506 and rapamycin binding protein (FKBP). In some embodiments, the ligand binding domain comprises calcineurinA (CNA) and a FK506 and rapamycin binding protein (FKBP). In some embodiments, the ligand binding domain comprises gibberellin insensitive (GIA) and gibberellin insensitive dwarf1 (GID1). In some embodiments, the ligand binding domain comprises Snap-tag and Halo tag. In some embodiments, the ligand binding domain comprises T14-3-3-cdeltaC and C-Terminal peptides of PMA2 (CT52). Further description of suitable ligand binding domain can be found in the art, e.g. WO2017091546.

### Intracellular Signaling Domain

In some embodiments, the intracellular signaling domain is inert to native ligand binding of the scaffold domain. In some embodiments, the intracellular signaling domain does not comprise an endogenous intracellular signaling domain of the scaffold domain. In some embodiments, when the scaffold domain is derived from a cytokine receptor, the intracellular signaling domain is inert to the corresponding cytokine bound by the cytokine receptor. In some embodiments, when the scaffold domain is derived from a cytokine receptor, the intracellular signaling domain does not comprise an endogenous intracellular signaling domain of the cytokine receptor. In some embodiments, when the scaffold domain is derived from an erythropoietin receptor (EpoR), the intracellular signaling domain is inert to erythropoietin. In some embodiments, when the scaffold domain is derived from an erythropoietin receptor (EpoR), the intracellular signaling domain does not comprise an endogenous intracellular signaling domain of the erythropoietin receptor (EpoR).

In some embodiments, the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway. In some embodiments, the intracellular signaling domain is selected from the group consisting of an intracellular signal transduction domain of IL-6RB (interleukin 6 receptor B), an intracellular signal transduction domain of FGFR1 (fibroblast growth factor receptor 1), and an intracellular signal transduction domain of VEGFR2 (vascular endothelial growth factor receptor 2). In some embodiments, the intracellular signaling domain is an intracellular signal transduction domain of IL-6RB and induces downstream signaling via the JAK/STAT signaling pathway. In some embodiments, the intracellular signaling domain is an intracellular signal transduction domain of FGFR1 and induces downstream signaling via the MAPK signaling pathway. In some embodiments, the intracellular signaling domain is an intracellular signal transduction domain of VEGFR2 and induces downstream signaling via the PLCG signaling pathway. In some embodiments, the intracellular signaling domain is an intracellular signal transduction domain of VEGFR2 and induces downstream signaling via the PI3K/Akt signaling pathways.

In some embodiments, the intracellular signaling domain comprises one or more modifications that modulate signaling activity of the intracellular signaling domain. In some embodiments, the modification comprises an amino acid insertion, an amino acid deletion, or an amino acid substitution. In some embodiments, the modification comprises an amino acid substitution. In some embodiments, the modification comprises a chemical modification. In some embodiments, the modification reduces negative feedback or reduces the cross-action of a secondary signaling pathway. In some embodiments, the modification comprises substitution of one or more tyrosine residues. In some embodiments, when the intracellular signaling domain is an intracellular signal transduction domain of IL-6RB, the intracellular signaling domain comprises a Y759A amino acid substitution. In some embodiments, when the intracellular signaling domain is an intracellular signal transduction domain of EGFR1, the intracellular signaling domain comprises a Y677F amino acid substitution. In some embodiments, when the intracellular signaling domain is an intracellular signal transduction domain of EGFR1, the intracellular signaling domain comprises a Y766F amino acid substitution. In some embodiments, when the intracellular signaling domain is an intracellular signal transduction domain of EGFR1, the intracellular signaling domain comprises a Y677F amino acid substitution and a Y766F amino acid substitution.

### Polynucleotides and Vectors

In another aspect, disclosed herein are isolated polynucleotides or sets of isolated polynucleotides encoding the chimeric ligand receptor. In some embodiments, the expression of the polynucleotide is under the control of a constitutively active promoter. In some embodiments, the expression of the polynucleotide is under the control of an inducible promoter. In some embodiments, the inducible promoter is a tetracycline-inducible promoter. Exemplary promoters for use in mammalian cells include but are not limited to cytomegalovirus (CMV) promoter, simian virus 40 (SV40) promoter, Rous sarcoma virus (RSV) promoter, elongation factor 1α (EF1α) promoter, and phosphoglycerate kinase (PGK) promoter.

In some embodiments, the polynucleotide encoding the chimeric ligand receptor is integrated into the genome by homologous recombination. In some embodiments, the polynucleotide encoding the chimeric ligand receptor is transiently transfected into the cells. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

### Cells

In another aspect, disclosed herein are genetically engineered cells comprising the polynucleotide or expressing the chimeric ligand receptor. In some embodiments, the cell is a mammalian cell. In some embodiments, the mammalian cell is a primary cell. In some embodiments, the mammalian cell is a cell line. In some embodiments, the mammalian cell a skin cell, a blood cell, a muscle cell, a bone cell, a neuronal cell, a fat cell, a liver cell, or a heart cell. In some embodiments, the cell is a stem cell. Exemplary stem cells include embryonic stem cells (ESCs), induced pluripotent stem cells (iPSCs), adult stem cells, and tissue-specific stem cells, such as hematopoietic stem cells (blood stem cells), mesenchymal stem cells (MSC), neural stem cells, epithelial stem cells, or skin stem cells. In some embodiments, the cell is an immune cell. Exemplary immune cells include T cells (e.g., helper T cells, cytotoxic T cells, memory T cells, regulatory T cells, natural killer T cells, and gamma delta T cells), B cells, natural killer (NK) cells, dendritic cells, macrophages, and monocytes. In some embodiments, the cell is a neuronal cell. Exemplary neuronal cells include neural progenitor cells, neurons (e.g., sensory neurons, motor neurons, cholinergic neurons, GABAergic neurons, glutamatergic neurons, dopaminergic neurons, or serotonergic neurons), astrocytes, oligodendrocytes, and microglia.

In some embodiments, the genetically engineered cell further comprises an engineered transgene. In some embodiments, the transgene comprises a synthetic promoter operably linked to a polynucleotide comprising a nucleic acid sequence encoding a target product. In some embodiments, the synthetic promoter is responsive to intracellular signaling from the chimeric ligand receptor. In some embodiments, the target product is selected from the group consisting of a therapeutic molecule (e.g., enzymes or antibodies), a prophylactic molecule, and a diagnostic molecule. In some embodiments, the target product is glucagon-like peptide 1, luciferase, secreted alkaline phosphatase (SEAP), or insulin.

In some embodiments, the genetically engineered cell comprises two or more chimeric ligand receptors. In some embodiments, the chimeric ligand receptors are each distinct from one another. In some embodiments, the chimeric ligand receptors each bind a different soluble ligand. In some embodiments, the genetically engineered cell further comprises two or more engineered transgenes. In some embodiments, each transgene comprises a synthetic promoter operably linked to a polynucleotide comprising a nucleic acid sequence encoding a target product. In some embodiments, each synthetic promoter is responsive to intracellular signaling from a distinct chimeric ligand receptor from the two or more chimeric ligand receptors expressed on the cell. In some embodiments, each target product is independently selected from the group consisting of a therapeutic molecule, a prophylactic molecule, and a diagnostic molecule.

### Methods

In another aspect, disclosed herein are methods of contacting the chimeric ligand receptor or the genetically engineered cell with a biological tissue or biological fluid. In some embodiments, the biological tissue or biological fluid is in a subject or is obtained from a subject. In some embodiments, the subject has been diagnosed with, is at risk of developing, or is suspected of having a medical condition. In some embodiments, the medical condition is a cancer or inflammatory condition.

In another aspect, disclosed herein are methods of activating a signaling pathway. The method comprises contacting the chimeric ligand receptor or the genetically engineered cell with a cognate ligand under conditions suitable for the chimeric ligand receptor to bind the cognate ligand, wherein binding of the cognate ligand with the chimeric ligand receptor induces a conformational reorganization of the multimerized scaffold domains that activates the intracellular signaling domains. In some embodiments, the method further comprises administering the cognate ligand to a surface of a cell.

In another aspect, disclosed herein are methods of producing a genetically engineered cell expressing a chimeric ligand receptor. The method comprises: synthesizing a chimeric ligand receptor expression vector encoding a chimeric ligand receptor comprising a scaffold domain capable of multimerizing and comprising an extracellular domain and a transmembrane domain, a ligand binding domain operably linked to the extracellular binding domain of the scaffold domain, and an intracellular signaling domain operably linked to the transmembrane domain of the scaffold domain, by fusing a first nucleic acid encoding the ligand binding domain to a second nucleic acid encoding the scaffold domain, and fusing the second nucleic acid with a third nucleic acid encoding the intracellular signaling domain; transfecting the chimeric ligand receptor expression vector into a cell; and inducing expression of the chimeric ligand receptor in the cell. In some embodiments, the method comprises transfecting the isolated polynucleotide or set of isolated polynucleotides or the vector or set of vectors into a cell; and inducing expression of the chimeric ligand receptor in the cell. In some embodiments, the method further comprises transfecting into the cell an isolated polynucleotide comprising a synthetic promoter operably linked to a nucleic acid sequence encoding a target product. In some embodiments, the synthetic promoter is responsive to intracellular signaling from the chimeric ligand receptor. In some embodiments, inducing expression of the chimeric ligand receptor comprises culturing the cell under conditions suitable for the cell to express the chimeric ligand receptor on a cell membrane of the cell.

### Examples

The following are examples of methods and compositions of the present disclosure. It is understood that various other embodiments may be practiced, given the general description provided herein.

Below are examples of specific embodiments for carrying out the claimed invention. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

Materials and Methods

HEK-293 Cell Culture and Transfection.

Human embryonic kidney cells (HEK-293T, American Type Culture Collection (ATCC): CRL-11268) were cultivated in Dulbecco's modified Eagle's medium (DMEM; 52100039, Thermo Fisher) supplemented with 10% (v/v) FBS (F7524, lot BCBS0318V, Sigma-Aldrich) and 1% (v/v) streptomycin/penicillin (L0022, Biowest) in a humidified atmosphere of 5% CO₂ in air at 37° C. For experiments, 1.5×10⁶ cells in 12 mL DMEM were seeded into 96-well plates (167008, Thermo Fisher) 24 h before transfection. The transfection mix in each well consisted of 125-130 ng of plasmid DNA mixed with 50 μL FBS and antibiotics-free DMEM and 600 ng of polyethyleneimine (24765-1, Polysciences, Inc.). The transfection mix was prepared separately, incubated for 20 min and then added to the cells for overnight transfection. In the morning, medium with the transfection mix was exchanged for 130 μL/well medium with different concentrations of the appropriate inducer or no inducer as a negative control. Reporter output was measured 24 h after induction for all experiments. Plasmid amounts used for transfections per set of six wells of a 96-well plate were as follows:

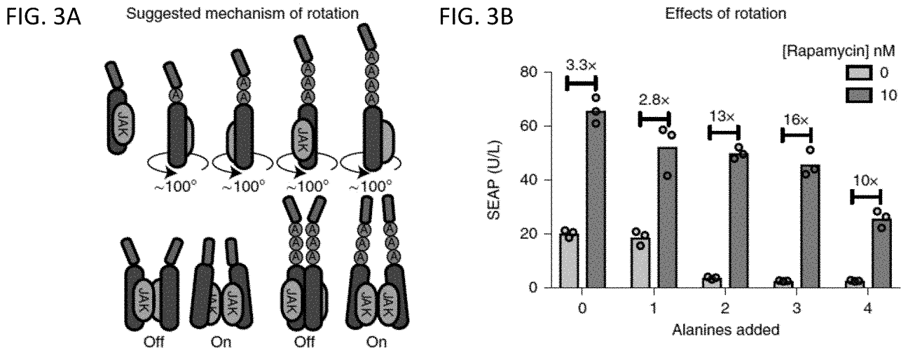

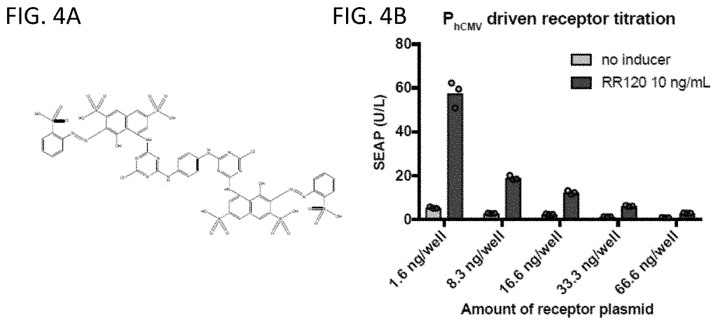

FIG. 3B; FIG. 4B: 5 ng for each receptor subunit or as indicated in the figure, 150 ng STAT3 reporter plasmid (pLS13), 25 ng STAT3 expression vector (pLS15) and 595 ng (or the appropriate amount to make a total of 775 ng) of a plasmid without mammalian promoter (pDF145).

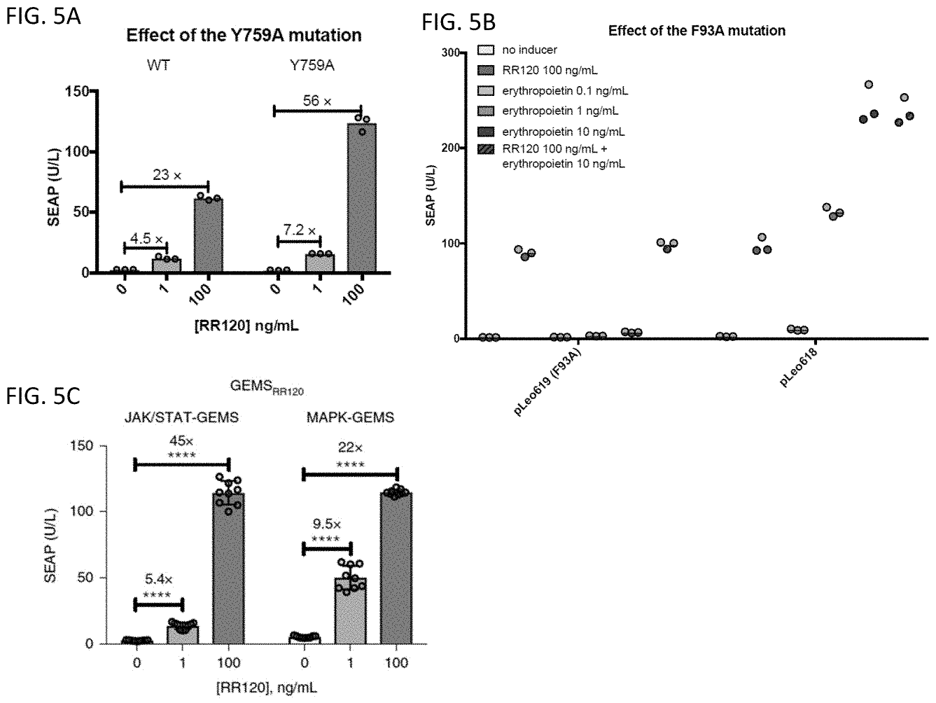

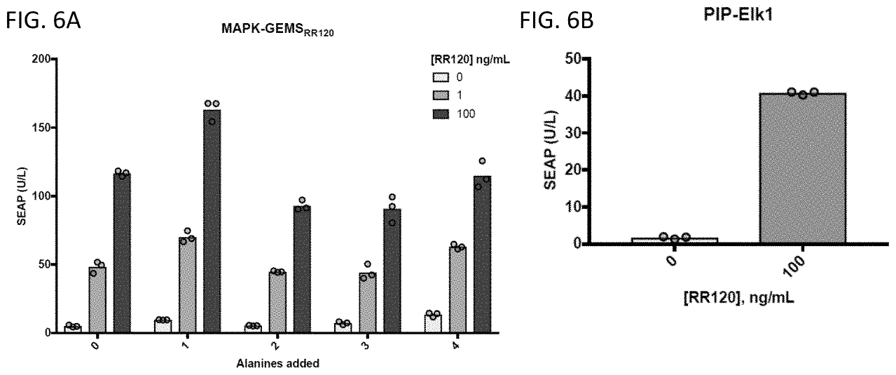

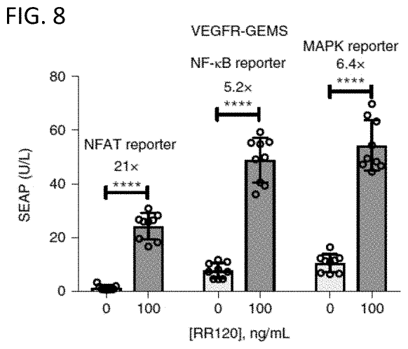

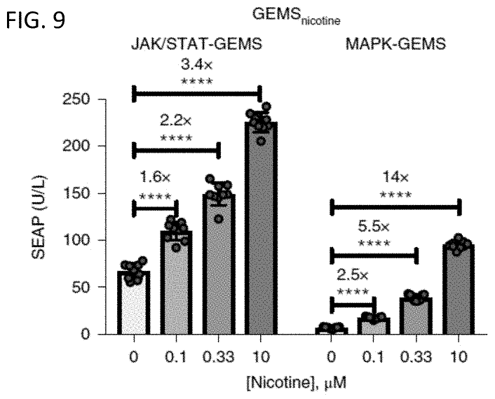

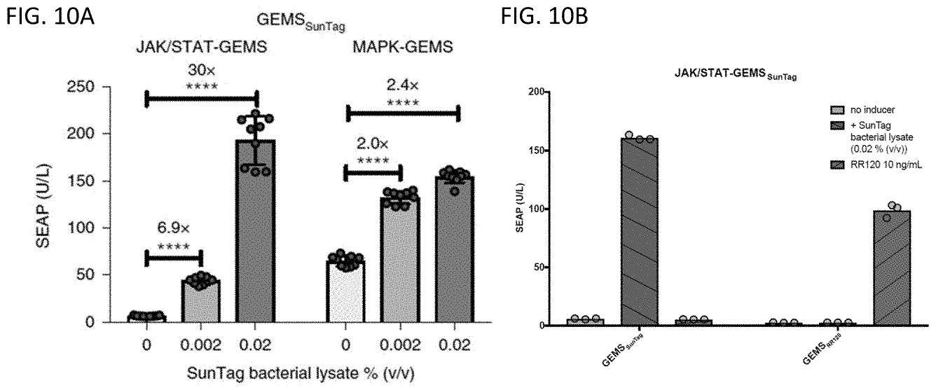

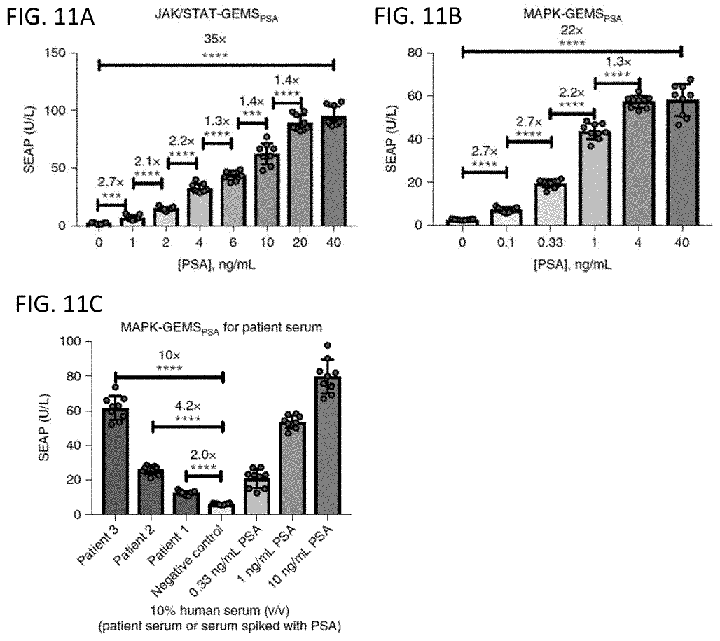

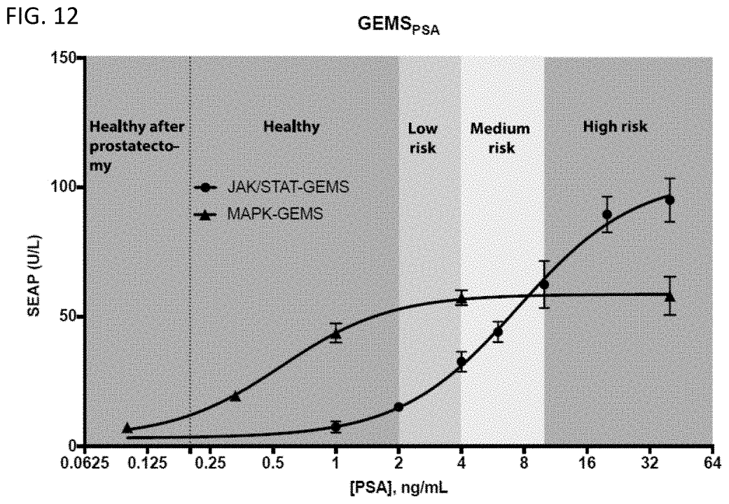

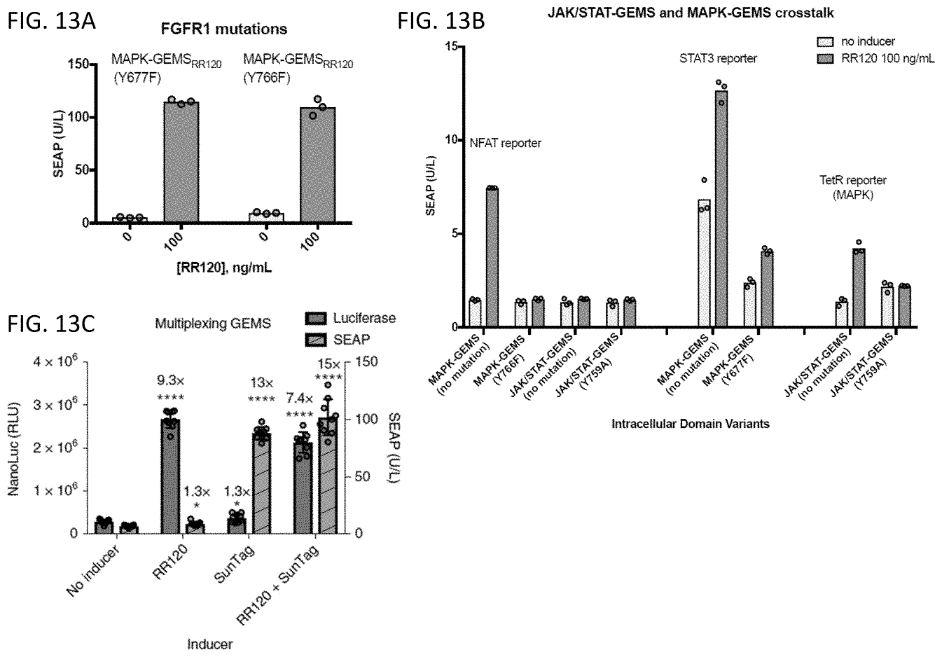

FIGS. 5A-C; FIG. 6A; FIG. 8; FIG. 9; FIGS. 10A-B; FIGS. 11A-C; FIG. 12; FIGS. 13A-B: 600 ng of receptor plasmids (for heterodimeric receptors 300 ng of each subunit) and for STAT readout 150 ng of a STAT3 reporter plasmid (pLS13) and 25 ng of a STAT3 expression vector (pLS15); for MAPK-readout 75 ng of a TetR reporter plasmid (pMF111) and 100 ng of a TetR-Elk1 fusion expression vector (MKp37); for NFAT readout 175 ng of a NFAT reporter plasmid (pHY30) and for NF-κB readout 175 ng of a NF-κB reporter plasmid (pKR32).

FIG. 6B: 600 ng of receptor plasmids and for MAPK-readout 150 ng of a PIP reporter plasmid (pMF199) and 25 ng of a PIP-Elk1 expression vector (pAT13).

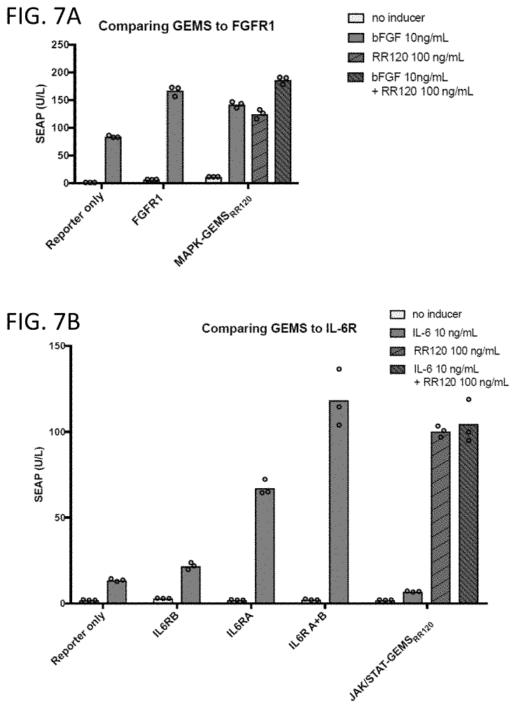

FIGS. 7A-7B: 600 ng of receptor plasmids for homodimeric receptors, 300 ng for each subunit of heterodimeric receptors or 600 ng pDF145 when no receptor was used. For STAT readout 150 ng pLS13 and 25 ng pLS15; for MAPK readout 75 ng pMF111 and 100 ng MKp37.

FIG. 13C: 200 ng of MAPK-GEMS_{RR120} (Y677F) (pLeo693) and 200 ng of JAK/STAT-GEMS_{SunTag} (pLeo620). For multiplexed STAT/MAPK readout 150 ng pLS13, 25 ng pLS15, 75 ng TetR NanoLuc reporter (pLeo665) and 100 ng MKp37.

Hybridoma Cell Culture and Transfection.

WEN1.3 cells (See Pogson M et al., Nat. Commun., 2016, 7:12535) were cultivated in DMEM (52100039, Thermo Fisher) supplemented with 10% (v/v) heat-inactivated FBS (F7524, lot BCBS0318V, Sigma-Aldrich), 1% (v/v) streptomycin/penicillin (L0022, Biowest), 10 mM HEPES buffer (Ser. No. 15/630,056, Thermo Fisher), and 50 mM 2-mercaptoethanol (M3148, Sigma-Aldrich) in a humidified atmosphere of 5% CO₂ in air at 37° C. Cells were transfected by electroporation with program CQ-104 of the 4D-Nucleofector System (Lonza) and the SF Cell Line 4D-Nucleofector X Kit L (V4XC-2024, Lonza).

For each transfection 1×10⁶ cells were washed twice by centrifugation for 5 min at 90 g and then resuspended in 1 mL of Opti-MEM I Reduced Serum Medium (31985062, Thermo Fisher); after washing, the cells were resuspended in 100 μL Lonza SF buffer containing 0.5 μg sleeping beauty transposase expression plasmid (pCMV(CAT)T7-SB100) and 9.5 μg of a plasmid for stable integration and expression of JAK/STAT-GEMS (pLeo695) or MAPK-GEMS (pLeo694). After pulsing, 500 μL prewarmed culture medium was added for 10 min. Cells were transferred to 1 mL culture media in 24-well plates (142475, Thermo Fisher) and were grown for 48 h. For creating stable cell lines, cells were transferred to 3 mL culture media in 6-well plates (140675, Thermo Fisher) and were selected with 5 μg/mL puromycin (ANT-PR-1, InvivoGen) for 14 d. For experiments, cells were centrifuged for 5 min at 90 g and resuspended in culture media (with or without RR120) to give a concentration of 1×10⁶ cells/mL, and then 500 μL of the cell suspension was transferred per well of a 48-well plate. IL-10 ELISAs (900-TM53, PeproTech) were performed 24 h after induction.

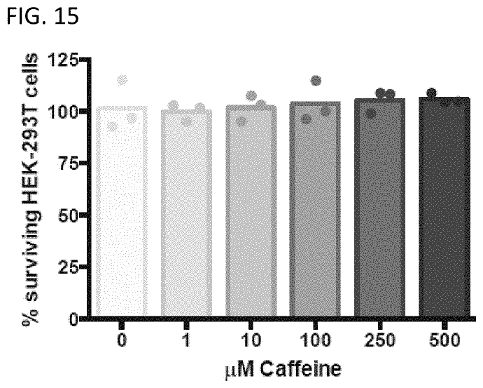

Cell Culture (FIGS. 15-34).

Human embryonic kidney cells (ATCC: CRL3216, HEK-293T) and adipose tissue-derived human telomerase reverse transcriptase-immortalized human mesenchymal stem cells (ATCC: SCRC4000, hMSC-hTERT) were cultured in Dulbecco's modified Eagle's medium (DMEM; Life Technologies, Carlsbad, Calif., USA) supplemented with 10% (v/v) fetal calf serum (FCS; BioConcept, Allschwil, Switzerland; lot no. 022M3395) and 1% (v/v) penicillin/streptomycin solution (Sigma-Aldrich, Munich, Germany). All cells were cultured in a humidified atmosphere containing 5% CO₂ at 37° C. Cell viability and number was assessed with an electric field multi-channel cell-counting device (CASY Cell Counter and Analyzer Model TT; Roche Diagnostics GmbH, Basel, Switzerland). For transfection in a 24-well plate format, 500 ng of plasmid DNA were diluted in 50 μL FCS-free DMEM, mixed with 2.5 μL polyethyleneimine (PEI; Polysciences Inc.; 1 mg mL-1), and incubated at room temperature for 20 min. Then, the transfection mixture was added dropwise to 1.25×10⁵ cells seeded 12 h before transfection. Twelve hours after transfection, the transfection medium was replaced by standard culture medium or medium supplemented with caffeine (cat. No. C0750, Sigma-Aldrich) or caffeine-containing compounds. Transgene expression was profiled 24 h later.

Generation of Genetically Stable Designer Cell Lines.

To develop stable designer cell lines according to the Sleeping Beauty transposon protocol (See Mates L et al., Nat. Genet., 2009, 41:753-761), one well of a 6-well plate with HEK-293T cells was co-transfected with pDB326 (1900 ng)/pSB100× (100 ng). After 12 h, the transfection medium was exchanged for standard culture medium. After an additional 24 h, the medium was exchanged for standard culture medium supplemented with 1 μg mL⁻¹ puromycin (cat. no. A1113803; ThermoFisher Scientific, Reinach, Switzerland) and a polyclonal cell population (C-STAR_{DB1}) was selected for 2 weeks. Subsequently, single cells were sorted by FACS according to fluorescence intensity, and single clones were grown in conditioned HEK-293T culture medium. Monoclonal cell populations were screened for caffeine-responsive SEAP expression and C-STAR_{DB3} was chosen as the best performer. The polyclonal stable cell line C-STAR_{DB6} was similarly generated with the plasmid pDB387, using 100 μg mL⁻¹ zeocin (cat. no. R25005; ThermoFisher Scientific, Reinach, Switzerland) as the selecting reagent.

Patient Serum.

Patient serum samples were a gift of C. Rentsch from the University Hospital of Basel. These samples were leftover samples from a previous study that was approved by the local ethical commissions in Basel and Bern, Switzerland, (EKBB 37/13). PSA concentrations were determined by ELISA (Human Total Prostate Specific Antigen ELISA Kit (ab188388), Abcam) according to the manufacturer's instructions. Cells expressing MAPK-GEMS_{PSA} were induced with 100 μL medium supplemented with 10% patient serum and reporter output was measured 24 h after induction.

mCherry-SunTag Production.

SunTag-mCherry was expressed in BL21 (DE3 pLys) E. coli. Bacteria were grown overnight at 37° C. Stationary bacterial culture (2 mL) was inoculated into 15 mL of Luria-Bertani broth. IPTG was added (final concentration: 0.5 mM) to induce gene expression. Cultures were harvested after incubation for 24 h at 25° C. Bacteria were collected by centrifugation (7,000 g for 10 min), resuspended in 50 mM Tris buffer pH 7.5, 100 mM NaCl, and 1 mM DTT and lysed by sonication (Diagenode Bioruptor, high power) on ice for 10 min (cycles of 10 s on/10 s off). Debris was removed by centrifugation (16,000 g, for 5 min). The supernatant was subsequently filtered through a 0.22 μm pore size PTFE filter (Sarstedt AG & Co). The resulting crude lysate was visibly red and was used for cell culture experiments without further purification.

Inducer Preparation.

bFGF (100-18B, PeproTech) and human IL-6 (200-06, PeproTech) were diluted in H₂O to 10 μg/mL. Nicotine (N3876, Sigma-Aldrich) was diluted to 200 mM in ethanol. PSA (497-11, Lee Bioscience, Lot W226903, 3.2 mg/mL) was directly used as a stock solution. Rapamycin (1292, Tocris) was prepared as a 1 mM solution in isopropanol. RR120 (R0378, dye content ≥50%, Sigma) was diluted in H₂O to 1 mg/mL. Working solutions were prepared by serial dilution in full DMEM directly before induction.

SEAP Measurement.

SEAP concentrations in cell culture supernatants were quantified in terms of absorbance increase due to hydrolysis of para-nitrophenyl phosphate (pNPP). 80 μL of heat-inactivated (30 min at 65° C., then centrifuged for 1 min, 3,220 g) supernatant was mixed in a 96-well dish with 100 μL of 2×SEAP buffer (20 mM homoarginine, 1 mM MgCl₂, 21% (v/v) diethanolamine, pH 9.8) and 20 μL of substrate solution containing 20 mM pNPP (Acros Organics BVBA). Samples were measured at 405 nm with a Tecan Genios PRO multiplate reader (Tecan AG) or an EnVision 2104 multilabel reader (PerkinElmer). SEAP production in vivo was quantified with the chemiluminescence SEAP reporter gene assay (cat. no. 11779842001, Sigma-Aldrich) according to the manufacturer's instructions.

NanoLuc Measurement.

Nanoluc concentrations in cell culture supernatants were quantified with the Nano-Glo Luciferase Assay System (N1110, Promega). 7.5 μL of sample was incubated for 5 min in 384-well plates (781076, Greiner Bio One) with 7.5 μL buffer/substrate mix (50:1), and luminescence was measured with a Tecan Genios PRO multiplate reader (Tecan AG).

CCK-8 Assay.

Cell viability was quantified with the Cell Counting Kit-8 (CCK-8; Dojindo Laboratories; cat. no. CK04) according to the manufacturer's instructions in Corning® 96 black well plates with a clear bottom (cat. no. CLS3603, Sigma-Aldrich). Briefly, 12 h after transfection, standard culture medium supplemented with or without caffeine was added to the cells. After 24 h, the medium was exchanged for standard culture medium supplemented with 10% (v/v) Cell Counting Kit-8. After an incubation period of one hour at 37° C., absorbance was measured at 450 nm with an EnVision 2104 multilabel reader (PerkinElmer), yielding a surrogate for cell viability.

Mouse IgG ELISA.

Mouse IgG levels in samples containing shGLP1-mIgG were quantified using the Mouse IgG ELISA Kit (cat. no. E-90G, ICL Lab), according to the manufacturer's instructions. The absorbance was quantified at 450 nm with an EnVision 2104 multilabel reader (PerkinElmer) and the mouse IgG levels were interpolated with a standard curve.

Glucose Tolerance Test.

Mice were challenged by intraperitoneal injection of glucose (2 g kg⁻¹ body weight in H₂O) and the glycemic profiles were generated by measurement of blood glucose levels with a glucometer (Contour® Next; Bayer HealthCare, Leverkusen, Germany) every 15 or 30 min for 120 min.

Insulin ELISA.

Insulin blood levels in tested mice were assessed with the Ultrasensitive Mouse Insulin ELISA (cat. no. 10-1132-01, Mercodia) according to the manufacturer's instructions. The absorbance was quantified at 450 nm with an EnVision 2104 multilabel reader (PerkinElmer).

shGLP-1 ELISA.

Blood levels of GLP-1 in tested mice were measured with the High Sensitivity GLP-1 Active ELISA Kit, Chemiluminescent (cat. no. EZGLPHS-35K, Merck) according to the manufacturer's instructions. The absorbance was quantified at 450 nm with an EnVision 2104 multilabel reader (PerkinElmer).

Caffeine Samples.

Coffee (Nespresso Grand Cru®) and tea samples (Cuida Te®) were prepared on a Nespresso Capri Automatic Sand machine (Koenig®). Starbucks coffee samples were obtained from a local Starbucks®. CocaCola® and Red Bull® samples were purchased from a local supermarket. Nesquik® (Nescafé Dolce Gusto®) was prepared on a Circolo Automatic EDG605B EX:1 (Nescafé Dolce Gusto®, DeLonghi). Military Energy Gum® (MarketRight Inc.) was mechanically crushed, covered with 40 mL water, and shaken for several hours at 37° C. to simulate chewing. Unless indicated otherwise, volumes of prepared beverages were those recommended by the respective manufacturer. All samples were diluted 1:50,000 in standard culture medium and added to the designer cells for quantification of caffeine.

Animal Experiments.

Encapsulated HEK-293T and C-STAR-derivative cells for the intraperitoneal implants were generated with an Inotech Encapsulator Research Unit IE-50R (EncapBioSystems Inc., Greifensee, Switzerland). Coherent alginatepoly-(L-lysine)-beads (400 μm diameter, 500 cells per capsule) were generated with the following parameters: 200-μm nozzle with a vibration frequency of 1025 Hz; 25-mL syringe operated at a flow rate of 410 units; 1.12 kV bead dispersion voltage (See Ye H et al., Proc. Natl. Acad. Sci. USA, 2013 110:141-146). Female C57BL/6 (14 weeks old) or T2D mice were injected with 1-2 mL of serum-free DMEM containing 1×10⁴ capsules. As genetically disposed T2D mice, db/db mice (female, 8 weeks old) were purchased from Janvier Labs. For the diet-induced obesity (DIO) model of T2D, C57BL/6 J mice (Janvier Labs, female, 4 weeks old) were fed for 10 weeks with a 10-kcal % or a 60-kcal % fat diet (TestDiet, cat. no. T-58Y1-58126) before C-STAR-controlled treatment. Blood glucose concentration was measured with a glucometer (Contour® Next; Bayer HealthCare, Leverkusen, Germany). Serum was collected using microtainer serum separating tubes (cat. no. 365967; Becton Dickinson, Plymouth, UK) according to the manufacturer's instructions. Experiments involving animals were carried out in accordance with the directive of the European Union by Ghislaine Charpin-El Hamri (No. 69266309; project No. DR2013-01 (v2)) at the Institut Universitaire de Technologie, UCB Lyon 1, F-69622 Villeurbanne Cedex, France.

Plasmid Preparation.

Plasmids were generated by digestion with standard restriction enzymes (New England BioLabs; HF enzymes were used whenever possible) and ligation with T4 DNA ligase (New England BioLabs). PCRs were performed with Q5 High-Fidelity DNA Polymerase (New England BioLabs) according to the manufacturer's instructions. For whole plasmid PCRs, elongation times were increased to 4 min/cycle.

Statistics.

Statistical analysis was done with GraphPad Prism 7. D'Agostino & Pearson normality tests were performed, confirming a Gaussian distribution of values in tested groups. Equal variance between groups was not assumed. For indicated experiments, Welch's two-sided t-tests were performed on n=9 biologically independent samples to identify significant induction of reporter gene expression in response to varying inducer concentrations. Graphs show the mean±s.d. as a bar diagram overlaid with a dot plot of individual data points. No adjustments were made for multiple comparisons. Representative graphs showing n=3 biologically independent samples are presented as bar diagrams overlaid with dot plots of individual data points, and no statistical analysis was performed. Exact P values, Welch corrected t-values and Welch corrected degrees of freedom for the analyzed data sets are provided in Table 1.

### Example 1: Design of the GEMS System

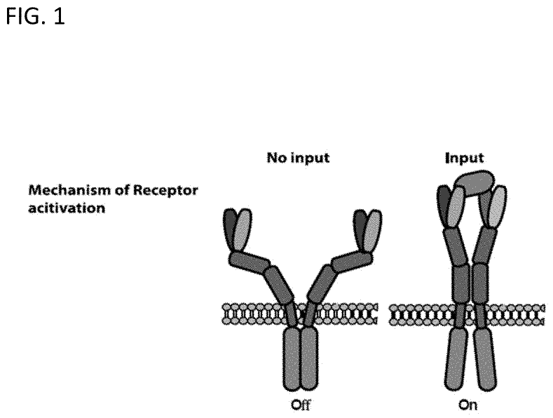

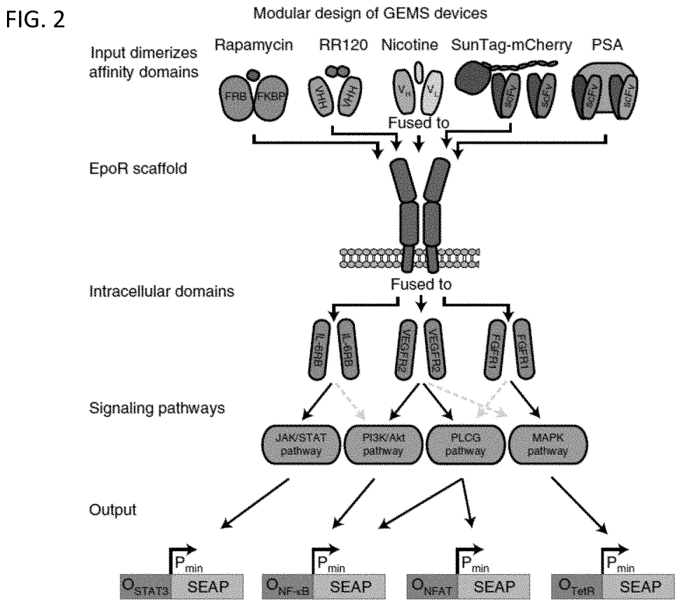

The GEMS system functions by the well-investigated mechanism of dimerization of extracellular receptor domains, which causes activation of intracellular signaling domains (FIG. 1). Cytokine receptors have a modular structure that tolerates the combination of intracellular and extracellular domains of different receptors to produce functional chimeras (See Arber C et al., Curr. Opin. Biotechnol., 2017, 47:92-101; Kawahara M & Nagamune T, Curr. Opin. Chem. Eng., 2012, 1:411-417). Inactive EpoR dimers are locked by transmembrane helix interactions in a conformation that prevents downstream signaling (See Seubert N et al., Mol. Cell, 2003, 12:1239-1250). Ligand binding to the receptors is thought to rotate each receptor subunit around its own axis and is likely accompanied by an increase in the distance between intracellular domains. The combination of these effects triggers downstream signaling (See Pang X & Zhou HX, PLoS Comput. Biol., 2012, 8:e1002427). We introduced a mutation into the erythropoietin receptor to render it inert to erythropoietin and fused it to affinity domains such as antibody fragments that dimerize in the presence of target molecules. As shown in FIG. 2, the EpoR transmembrane domain was fused to the intracellular signal transduction domains of IL-6RB (interleukin 6 receptor B), FGFR1 (fibroblast growth factor receptor 1) or VEGFR2 (vascular endothelial growth factor receptor 2). Once activated, these intracellular domains induce downstream signaling via JAK/STAT (Janus kinase/signal transducer and activator of transcription; induced by IL-6RB), MAPK (mitogen-activated protein kinase; induced by FGFR1) and PLCG (phospholipase C gamma; induced by VEGFR2), as well as PI3K/Akt (phosphatidylinositol 3-kinase/protein kinase B; induced by VEGFR2). Minimal promoters that are selectively responsive to the indicated pathways were used to rewire signaling to transgene expression.

### Example 2: Design of the Receptor Scaffold

The well-studied FRB/FKBP (FKBP-rapamycin binding protein/FK506 and rapamycin binding protein) system for rapamycin-induced dimerization (See Liu W et al., Biotechnol. Bioeng., 2008, 101:975-984) was used for initial characterization of the GEMS system. FRB and FKBP proteins were fused to the erythropoietin receptor extracellular domain, and its transmembrane domain was C-terminally linked to the JAK/STAT signaling domain of IL-6RB. Activated JAKs phosphorylate STAT3, which functions as transcription factor. Receptor activation can be quantified through the reporter protein SEAP (human placental secreted alkaline phosphatase) expressed from the reporter plasmid pLS13 (0Stat3-PhCMVmin-SEAP-pA) containing STAT3-binding sites 5′ of a minimal promoter (See Schukur L et al., Sci. Transl. Med., 2015, 7:318ra201). Up to four alanine residues were added between the transmembrane domain of EpoR (EpoR0-4A) and the intracellular domain of IL-6RB (IL-6RBint). Each additional alanine elongates the transmembrane helix and rotates the intracellular domain by approximately 100° relative to the extracellular domain (See Seubert N et al., Mol. Cell, 2003, 12:1239-1250; Liu W et al., Biotechnol. Bioeng., 2008, 101:975-984). This was done to identify a receptor conformation with minimal JAK activation in the off state (FIG. 3A). FRB/FKBP receptors (pTS1000-pTS1009, PhCMV-FRB/FKBP-EpoR0-4A-IL-6RBint-pA) were responsive to induction by rapamycin, and the number of alanine residues influenced receptor performance. Basal gene expression was highest for the variants with zero or one alanine, suggesting insufficient inhibition of preformed dimers. The highest fold change from induced to uninduced SEAP expression was observed for the variant with three alanine residues (FIG. 3B), which was adopted as a template for further experiments.

### Example 3: Validation and Optimization of the GEMS Receptor Scaffold

To validate and optimize the receptor scaffold, we focused on the azo dye RR120 (reactive red 120) as a ligand to showcase that the scaffold can be used in the context of environmental sensors for synthetic molecules. RR120 is a large, dimeric, hydrophilic molecule (FIG. 4A) that has until now been undetectable by biological systems. FRB/FKBP proteins were exchanged for VHH_{A52} (camelid heavy chain antibody A52) raised against RR120 that was shown to dimerize in the presence of RR120 (See Spinelli S et al., J. Mol. Biol., 2001, 311:123-129). The linker region between antibody and EpoR consists of the amino acids ser-gly-glu-phe and should provide rotational freedom to facilitate antibody binding. The resulting homodimeric receptors (plasmid name: pLeo615, function determining plasmid elements: P_{hCMV}-VHH_{A52}-EpoR_{3A}-IL-6RBᵢₙₜ-pA) could be triggered with low nanomolar concentrations of RR120 (FIG. 4B). Transfecting plasmids for P_{hCMV} (human cytomegalovirus immediate early promoter)-driven receptor expression reduced reporter secretion (FIG. 4B), and the P_{hCMV} promoter was therefore exchanged for a weaker simian virus 40-derived promoter (P_{SV40}) for further experiments (pLeo617, P_{SV40}-VHH_{A52}-EpoR_{3A}-IL-6RBᵢₙₜ-pA). The signal-to-noise ratio was further improved by mutating Y759 (tyrosine 759) of IL-6RBᵢₙₜ to alanine. Mutations of Y759 are found in various cancers and inflammatory diseases, as phosphorylated tyrosine Y759 is a binding site for proteins that negatively regulate JAK/STAT signaling (See Silver J S & Hunter C A, J. Leukoc. Biol., 2010, 88:1145-1156). Therefore, we generated IL-6RBₘ (modified IL-6RBᵢₙₜ) with the Y759A substitution to modulate the signaling activity. Total SEAP expression increased in receptors carrying the mutation, with only minor increases in uninduced reporter production (FIG. 5A; pLeo618, P_{SV40}-VHH_{A52}-EpoR_{3A}-IL-6RBₘ-pA). To render the receptor insensitive to its native ligand erythropoietin, the mutation F93A (See Middleton S A et al., J. Biol. Chem., 1996, 271:14045-14054) was introduced in the main erythropoietin binding site of EpoR3A (EpoRₘ; FIG. 5B). This is critical for clinical settings, in which receptor function must be tightly regulated and be independent of host factors. Receptor optimization robustly improved the ratio of induced versus noninduced SEAP secretion to over 40-fold (FIG. 5C, left panel; pLeo619, P_{SV40}-VHH_{A52}-EpoRₘ-IL-6RBₘ-pA). Combination of the EpoR3A variation that minimizes uninduced receptor activation with a mutation of Y759 that reduces negative feedback appears to act synergistically to enhance the signal-to-noise ratio, resulting in the observed high switching performance. Therefore, we adopted this optimized receptor scaffold structure as the standard GEMS platform; the GEMS device targeting RR120 was designated as GEMS_{RR120}.

### Example 4: Rerouting Input to Different Intracellular Signaling Pathways

To broaden the applicability of GEMS, we adapted GEMS_{RR120} (pLeo619) to other dimerization-dependent signaling pathways. GEMS_{RR120} was rerouted to signal via the MAPK pathway by exchanging the IL-6RB intracellular domain for the intracellular domain of FGFR1 (fibroblast growth factor receptor 1), which functions via dimerization-dependent signaling (See Reichhart E et al., Angew. Chem. Int. Ed. Engl., 2016, 55:6339-6342). MAPK-GEMS_{RR120} (MAPK-dependent GEMS_{RR120}) was rewired to drive reporter gene expression with a TetR-Elk1 fusion protein (MKp37, P_{hCMV}-TetR-ELK1-pA) and a TetR-dependent reporter plasmid (pMF111, O_{Tet}-P_{hCMVmin}-SEAP-pA) (FIG. 5C, right panel). We assumed that signaling dynamics could be modulated in a similar manner to that adopted for the JAK/STAT pathway-dependent receptors by inserting 0-4 alanine residues C-terminal to the EpoRₘ transmembrane domain (pLeo628, pLeo642, pLeo643, pLeo644, pLeo645, P_{SV40}-VHH_{A52}-EpoR_{m0-4A}-FGFR1ᵢₙₜ-pA), but we found that the number of alanine residues did not alter receptor activity as dramatically in this case (FIG. 6A). The variant without additional alanine residues (pLeo628) was chosen for further analysis. Interestingly, lower concentrations of RR120 could be detected with higher fold changes when using MAPK-GEMS_{RR120} compared to JAK/STAT-GEMS_{RR120} (FIG. 5C). TetR-Elk1 is a synthetic mammalian transcription factor containing a sequence-specific DNA binding domain (TetR) and a transcription-activation domain (Elk1) (See Keeley M B et al., Biotechniques, 2005, 39:529-536). To demonstrate the versatility of transcription factors of this type, we replaced TetR with another sequence-specific DNA binding domain, PIP (pristinamycin-induced protein (See Fussenegger M et al., Nat. Biotechnol., 2000, 18:1203-1208); pAT13, P_{hCMV}-PIPELK1-pA), and validated its performance using MAPK-GEMS_{RR120} and the cognate PIP-specific reporter plasmid (pMF199, O_{PIP}P_{hCMVmin}-SEAP-pA) (FIG. 6B). To benchmark GEMS performance with the parental native receptors, we transfected FGFR1 or IL-6R into HEK-293 cells and activated them with bFGF (basic fibroblast growth factor) or IL-6, respectively. In both cases, the resulting reporter gene expression reached levels that were comparable to those obtained with the corresponding GEMS receptors, confirming that GEMS-activated signaling matched the performance of endogenous receptor signaling (FIGS. 7A-B). To explore whether the GEMS platform is compatible with further signaling pathways and to confirm its broad applicability, we engineered a GEMS variant containing the intracellular domain of VEGFR2 (vascular endothelial growth factor receptor 2; pLeo690, P_{SV40}-VHH_{A52}-EpoR_{m0A}-VEGFR2int-pA). VEGFR2 is known to activate MAPK and PI3K/Akt (phosphatidylinositol 3-kinase/protein kinase B) as well as PLCG (phospholipase C gamma) (See Abhinand C S et al., J. Cell Commun. Signal., 2016, 10:347-354). Indeed, VEGFR2-GEMS signaling was successfully rerouted to NFAT-(pHY30, O_{NFAT}-P_{hCMVmin}-SEAP-pA) and NF-κB-(pKR32, O_{NF-κB}-P_{hCMVmin}-SEAP-pA) driven target gene expression (FIG. 8).

### Example 5: Targeting GEMS for Nicotine (GEMS_{nicotine})

To examine the generality of GEMS, we next focused on nicotine as a pharmacologically active small molecule and re-engineered the system for nicotine input. Hapten responsiveness was created by cloning V_{H} and V_{L} (variable fragments of heavy and light chains, respectively) of the nicotine antibody mAb-Nic12 (See Tars K et al., J. Mol. Biol., 2012, 415:118-127) separately into the receptor framework of JAK/STAT-dependent and MAPK-dependent GEMS. This antibody was chosen because the crystal structure revealed that nicotine is deeply buried between the heavy and light chains of the Fab (fragment antigen binding). JAK/STAT- and MAPK-GEMSnicotine (pLeo626/pLeo627, pLeo667/pLeo668, PSV40-VH/VL-EpoRₘ-IL-6RBm/FGFR1int-pA) were significantly induced by nicotine at concentrations as low as 100 nM, which is a typical serum nicotine concentration reached after smoking a single cigarette or when nicotine replacement products are used (FIG. 9; see Benowitz N L et al., Handb. Exp. Pharmacol., 2009, 192:29-60).

### Example 6: Targeting GEMS for Extracellular Proteins (GEMS_{SunTag})

To see whether GEMS could also be adapted to sense extracellular proteins, a SunTag_{8×} (See Tanenbaum M E et al., Cell, 2014, 159:635-646), consisting of a string of eight GCN4 peptide tags was fused to mCherry and expressed in bacteria. We cloned the well-characterized GCN4-specific scFv (scFv_{αGCN4}) into the GEMS receptor framework to induce the dimerization or oligomerization of SunTag, and thus activate, the receptor. JAK/STAT-GEMS_{SunTag} and MAPK-GEMS_{SunTag} (pLeo620, pLeo669, P_{SV40}-scFv_{αGCN4}-EpoRₘ-IL-6RBₘ/FGFR1ᵢₙₜ-pA) were induced at dilutions of 1:5,000 (v/v) of crude bacterial lysate containing the SunTag_{8×}-mCherry fusion protein, allowing the use of lysate without prior purification (FIG. 10A). The same conditions did not induce reporter expression in other GEMS devices, confirming that cell signaling was induced by the SunTag and not by other substances in the lysate (FIG. 10B).

### Example 7: Targeting GEMS for PSA (GEMS_{PSA})

We tested whether the system could be adapted for sensing a monomeric protein. We chose PSA as a well-investigated and clinically important biomarker. PSA is an enzyme produced by the prostate and is used to screen for prostate cancer development. Prostate cancer is the most common cancer in males and is the cause of death in 1-2% of men (See Attard G et al., Lancet, 2016, 387:70-82). GEMS_{PSA} was designed with scFvs against non-overlapping epitopes that were expected to form heterodimeric receptors. Three antibodies, 8G8F5, 5A5 and 5D3D11 (See Ménez R et al., J. Mol. Biol., 2008, 376:1021-1033) shown by crystallization to bind distinct epitopes of PSA were converted to scFvs by adding a (GGGGS)₄ (SEQ ID NO: 27) linker sequence between V_{L} and V_{H}. These scFvs were cloned into the receptor framework with V_{H} fused to the EpoR extracellular domain. Three combinations of heterodimeric JAK/STAT-GEMS were tested. Among them, JAK/STAT-GEMS_{PSA} containing scFv_{8G8F5} (pLeo622, P_{SV40}-scFv_{8G8F5}-EpoRₘ-IL-6RBₘ-pA) and scFv_{5A5} (pLeo623, P_{SV40}-scFv_{5A5}-EpoRₘ-IL-6RBₘ-pA) generated robust PSA-dependent signaling (FIG. 11A). Combinations of either of the receptors with receptors containing scFv_{5D3D11} (pLeo621, P_{SV40}-scFv_{5D3D11}-EpoRₘ-IL-6RBₘ-pA) were not inducible with PSA.

JAK/STAT-GEMS_{PSA} (pLeo622/pLeo623) was measured to signal with an EC50 of 7.5±0.5 ng/mL (264±18 pM) and showed an almost linear response in the clinically important range of 4-10 ng/mL, referred to as diagnostic gray zone for PSA screening (FIG. 12; see Polascik T J, J. Urol., 1999, 162:293-306). The high signal-to-noise ratio of reporter expression allowed precise discrimination of diagnostically critical PSA concentrations with high significance (FIG. 11A). MAPK-GEMS_{PSA} with the same functional scFv pair (pLeo670/pLeo671, P_{SV40}-scFv_{8G8F5}/scFv_{5A5}-EpoRₘ-FGFR1ᵢₙₜ-pA) was more sensitive, with an EC50 of 0.55±0.03 ng/mL (FIG. 12), and could reliably sense PSA concentrations as low as 0.1 ng/mL (FIG. 11B). This might be important for patients treated with a radical prostatectomy, as biochemical recurrence is typically defined as PSA concentrations ≥0.2 ng/mL (See Cookson M S et al., J. Urol., 2007, 177:540-545).

The more sensitive MAPK-GEMS_{PSA} was used to classify serum of patients diagnosed with prostate cancer in comparison to negative control samples from patients who underwent radical prostatectomy (FIG. 11C). Reporter output was proportional to serum PSA levels determined by ELISA (enzyme-linked immunosorbent assay). This data confirms that human cells engineered for expression of GEMS are able to detect biomarkers in the clinically relevant concentration range.

### Example 8: Multiplexing MAPK-GEMS and JAK/STAT-GEMS

We examined whether different GEMS were compatible and could independently operate in the same cell to sense two different inputs and produce two different outputs. GEMS containing the intracellular domain of FGFR1 (pLeo628, MAPK-GEMS) predominantly activated MAPK signaling but also had a minor effect on NFAT and STAT3 signaling. However, introduction of a Y766F mutation in the PLCG-binding site of FGFR1 (pLeo692, MAPK-GEMS) as well as a Y677F mutation in the STAT binding site (pLeo693, MAPK-GEMS) abolished this crosstalk while maintaining the performance of the original MAPK signaling (FIG. 13A; see Ornitz D M & Itoh N, Rev. Dev. Biol., 2015, 4:215-266). Likewise, a Y759A mutation in the intracellular IL-6RB domain (pLeo619, JAK/STAT-GEMS) cancelled the interference with MAPK signaling while maintaining the original JAK/STAT signaling performance (FIG. 13B; see Silver J S & Hunter C A, J. Leukoc. Biol., 2010, 88:1145-1156). We replaced SEAP of the TetR-reporter plasmid with the secreted variant of the bioluminescent reporter protein NanoLuc (pLeo665, O_{Tet}-P_{hCMVmin}-SecNanoLuc-pA; see Hall M P et al., ACS Chem. Biol., 2012, 7:1848-1857) to provide two outputs that can be separately analyzed in the cell culture supernatant. MAPK-GEMS_{RR120} (Y677F) (pLeo693) and JAK/STAT-GEMS_{SunTag} (pLeo620) were co-transfected, and the resulting SEAP (STAT3 reporter) as well as NanoLuc (MAPK reporter) expression confirmed independent input-specific activation and orthogonal processing of the two GEMS signaling pathways (FIG. 13C).

### Example 9: GEMS Induce IL-10 Secretion in Hybridoma Cells

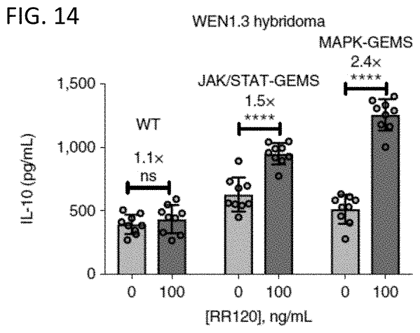

To test whether GEMS can tap into the signaling pathways of immune cells, MAPK-GEMS_{RR120} (pLeo694) and JAK/STAT-GEMS_{RR120} (pLeo695) were stably integrated into the hybridoma cell line WEN1.3 (See Ménez R et al., J. Mol. Biol., 2008, 376:1021-1033). Profiling of IL-10 secretion upon GEMS activation by RR120 confirmed that GEMS_{RR120} was tapping into the endogenous MAPK signaling, as well as JAK/STAT pathways, and substantially increased IL-10 secretion (FIG. 14).

### Example 10: Design of a Caffeine-Inducible Gene Switch

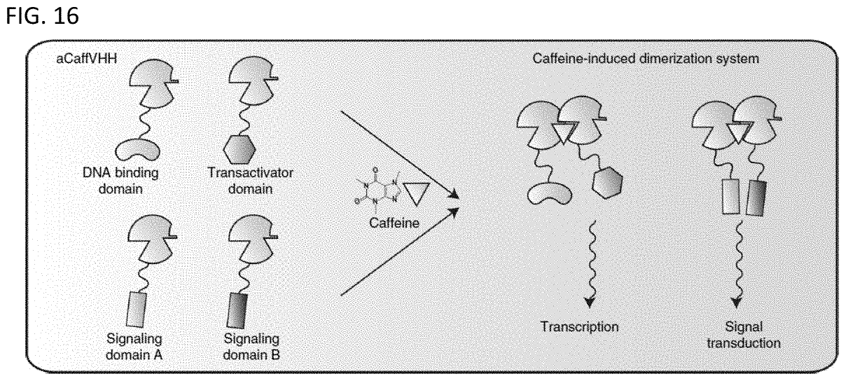

After drinking an average cup of coffee, blood levels of caffeine peak in the low micromolar range (See Noguchi K et al., J. Pharmacol. Sci., 2015, 127:217-222; Teekachunhatean S et al., ISRN Pharmacol., 2013, 2013:1-7), so for the present purpose, we required a caffeine sensor system for non-toxic (FIG. 15), physiologically relevant concentrations. To capture these concentrations, we established a caffeine-inducible protein dimerization system in mammalian cells to create different types of gene switches. (i) Fusion of the caffeine-binding single-domain antibody aCaffVHH to DNA-binding and transactivation domains reconstitutes synthetic transcription factors driving chimeric target promoters in a caffeine-responsive manner. (ii) Fusion of the caffeine-binding single-domain antibody aCaffVHH to intracellular signaling domains of different mammalian receptor classes reconstitutes synthetic signaling cascades and allows caffeine to dose-dependently activate different pathway-specific promoters (FIG. 16).

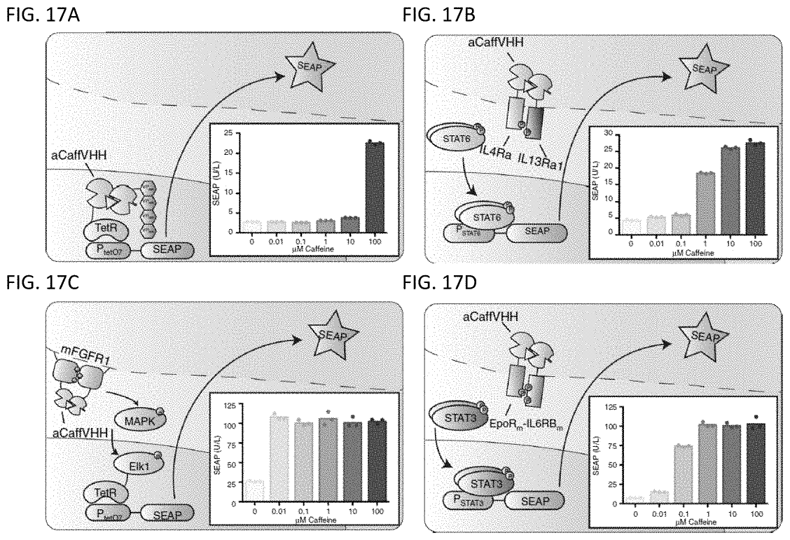

To design an aCaffVHH-dependent transcription factor-based gene switch, we C-terminally fused aCaffVHH to the DNA-binding TetR-domain (P_{SV40}-TetR-aCaffVHH-pA_{SV40}, pDB307), as well as N-terminally to four repeats of a transactivating 12-amino-acid peptide (VPₘᵢₙ, P_{CAG}-aCaffVHH-VP_{min×4}-pA_{βG}, pDB335). In this design, the presence of caffeine should dimerize the DNA-binding domain with the transactivating VPₘᵢₙ domain and lead to gene expression (FIG. 17A). Utilizing the reporter gene human placental-secreted alkaline phosphatase (SEAP) controlled by a TetR-dependent promoter (P_{tetO7}-SEAP-pA_{SV40}, pMF111), we observed clear caffeine-dependent gene expression in the presence of 100 μM caffeine (FIG. 17A).

We reasoned that this low sensitivity to caffeine might be due to the absence of signal amplification in this split transcription factor setup. Therefore, we applied the caffeine-inducible dimerization system to different signaling pathway-specific signal transduction domains. First, we fused aCaffVHH N-terminally to the transmembrane domain of interleukin 13 receptor subunit alpha 1 (IL13Rα1, P_{hCMV}-aCaffVHH-IL13Rα1-pA_{bGH}, pDB323), as well as interleukin 4 receptor subunit alpha (IL4Rα, P_{hCMV}-aCaffVHH-IL4Rα-pA_{bGH}, pDB324). Addition of caffeine should induce heterodimerization of these receptors and activate signal transducer and activator of transcription 6 (STAT6) signaling. Indeed, when we co-transfected STAT6 (P_{hCMV}-STAT6-pA_{bGH}, pLS16) and a STAT6-responsive reporter construct (P_{STAT6}-SEAP-pA_{SV40}, pLS12), we could see caffeine-dependent gene expression starting from 1 μM caffeine (FIG. 17B), a considerable improvement in sensitivity compared to the split transcription factor setup using pDB307 and pDB335. However, the absolute output strength of this setup in SEAP units was limited, necessitating a more powerful system.

To overcome the output strength issue, we fused aCaffVHH C-terminally to the intracellular part of the murine fibroblast growth factor receptor 1 (mFGFR1, P_{hCMV}-mFGFR1₄₀₅₋₈₂₂-aCaffVHH-pA_{bGH}, pDB395) (See Grusch M et al., EMBO J., 2014, 33:1713-1726). The presence of caffeine should homodimerize mFGFR1₄₀₅₋₈₂₂-aCaffVHH and lead to MAPK signaling, which we re-routed to TetR-dependent pMF111 by co-transfecting TetR-Elk1 TetR-Elk1-pAbGH, MKp37). The signal amplification of the MAPK signaling cascade (See Huang C Y & Ferrell J E, Proc. Natl Acad. Sci. USA, 1996, 93:10078-10083) yielded a strong and sensitive gene expression response in the presence of as little as 0.01 μM caffeine (FIG. 17C). However, this extraordinary sensitivity to caffeine may be detrimental in a therapeutic setting, since even trace amounts of caffeine would induce the gene circuit. Additionally, the requirement of the re-routing protein TetR-Elk1 meant that transfection of three plasmids was necessary for this system.

Improving on the mFGFR1-dependent system, we fused aCaffVHH N-terminally to an erythropoietin receptor derivative (EpoR, P_{hCMV}-aCaffVHH-EpoRₘ-IL-6RBₘ-pA_{bGH}, pDB306) (See Kawahara M et al., J. Biochem., 2001, 130:305-312; Scheller L et al., Nat. Chem. Biol., 2018, 14:723-729) leading to homodimerization of the receptor in the presence of caffeine and subsequent JAK/STAT signaling through STAT3. As HEK-293T cells endogenously express STAT3, we only needed to transfect pDB306 and a STAT3-dependent reporter plasmid (P_{STAT3}-SEAP-pA_{SV40}, pLS13). This setup yielded a strong and sensitive gene expression system with a maximal response at 1 μM caffeine (FIG. 17D).

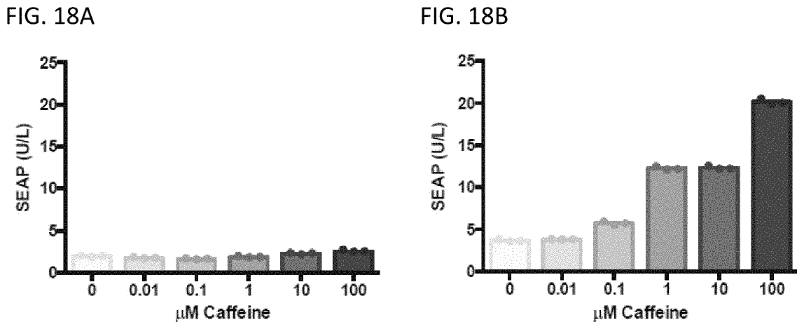

Overall, caffeine-dependent STAT3-signaling proved to be the best fit in terms of potency, sensitivity to physiological caffeine levels, and number of components, and so it was used for further experiments. Due to receptor homodimerization and endogenous STAT3 expression, we only needed to transfect two components to obtain a full C-STAR system. Since the presented gene expression systems had different sensitivities and relied on orthogonal promoters, they could be used for endowing designer cells with a nonlinear response to caffeine by expressing multiple receptors (FIGS. 18A-B).

### Example 11: Characterization of the Caffeine-Inducible C-STAR System

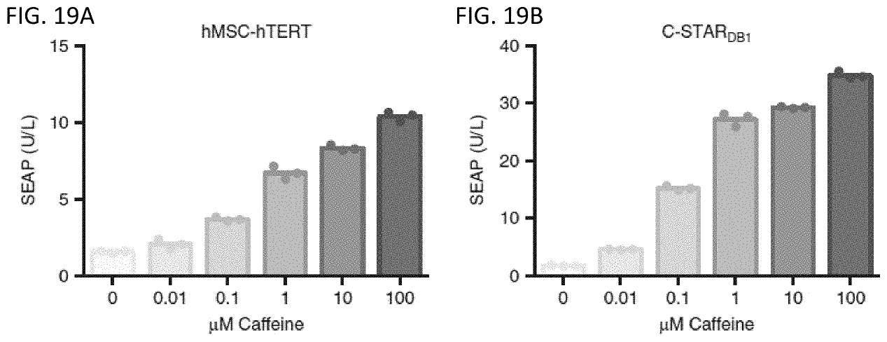

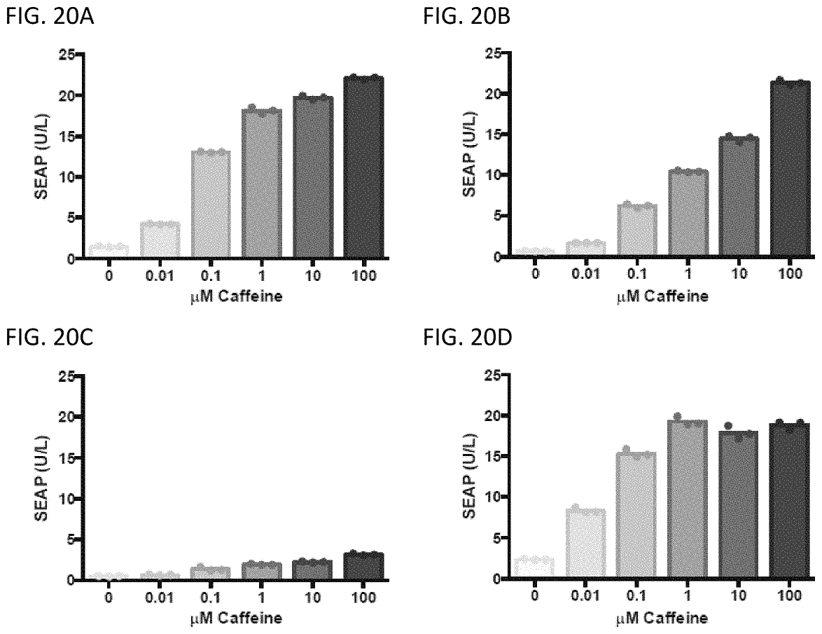

Functionality of the C-STAR system was also demonstrated in human telomerase reverse transcriptase-immortalized human mesenchymal stem cells (hMSC-hTERT) (FIG. 19A). However, HEK-293T cells showing higher caffeine sensitivity and protein secretion capacity were used in all follow-up experiments. For long-term experiments, the C-STAR receptor (P_{hEF-1α}-aCaffVHH-EpoRₘ-IL-6RBₘ-pA_{SV40}, pDB326) was stably integrated into the genome of HEK-293T cells, creating the designer cell line C-STAR_{DB1}. The caffeine dose-response relationship of this polyclonal cell line was similar to that of the transiently transfected cells (FIG. 19B). However, selection of monoclonal C-STAR cell lines yielded clones with different sensitivities for caffeine (FIGS. 20A-D). Further in vitro experiments were conducted with the C-STAR_{DB1} cell line.

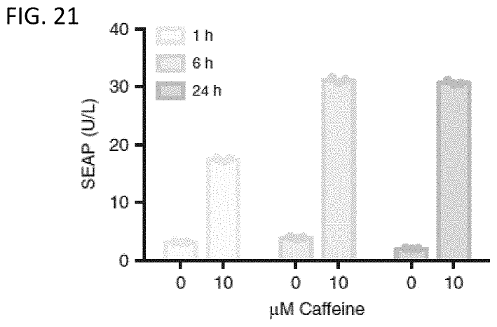

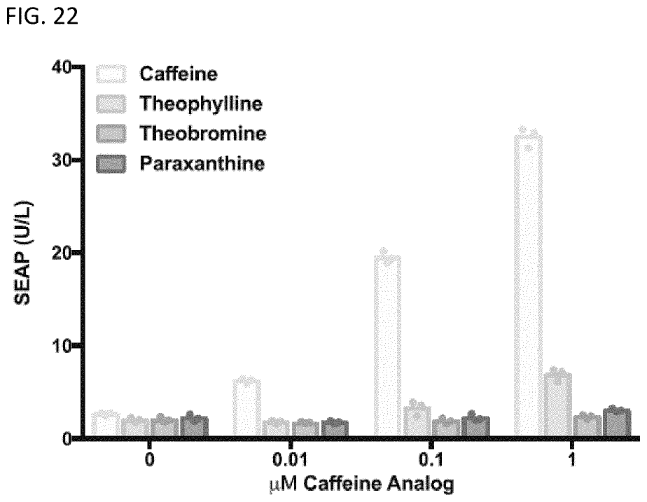

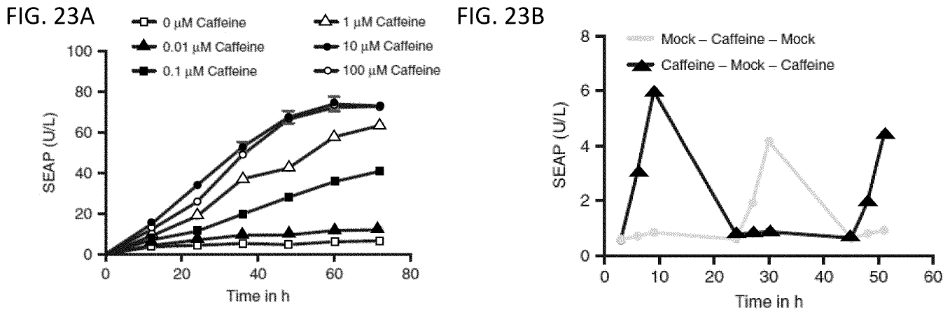

To capture the time window of high caffeine concentration in the blood, an in vivo C-STAR system would need to induce gene expression after brief exposure to the inducer. Exposure to physiologically relevant concentrations of caffeine induced a half-maximal response of the C-STAR system within just one hour, and a maximal response was obtained after six hours of exposure (FIG. 21). Since the half-life of caffeine in human blood is approximately five hours (See Teekachunhatean S et al., ISRN Pharmacol., 2013, 2013:1-7), in vivo activation of the C-STAR system by caffeine should be feasible. Among the caffeine analogs tested in vitro, only theophylline showed modest cross-activation of C-STAR at 1 μM concentration (FIG. 22), which is unlikely to be reached in the physiological situation (See Hicks M B et al., Food Res. Int., 1996, 29:325-330; Hackett J et al., J. Anal. Toxicol., 2008, 32: 695-701). The response time of the C-STAR system after caffeine addition was assessed and the C-STAR system responded in a timely manner to the presence of caffeine, yielding detectable amounts of reporter protein at 12 h, whereas no induction of SEAP expression was seen in the negative control lacking caffeine (FIG. 23A). Testing the reversibility of the gene circuit, C-STAR_{DB1} cells were incubated with physiologically relevant concentrations of caffeine or the equivalent amount of H₂O (mock), with an exchange of caffeine to mock, or vice versa, every day (FIG. 23B). The system was shut off by the removal of caffeine and could be activated again by the renewed addition of caffeine to the cells, indicating reversibility after removal or degradation of caffeine.

### Example 12: Caffeine Quantification in Commercial Beverages Using C-STAR

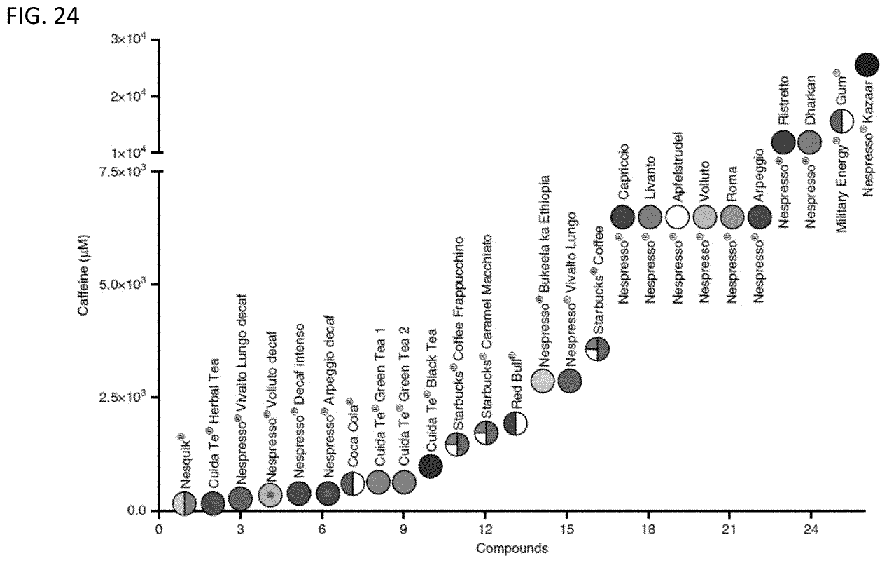

Caffeine is a component of various beverages. Therefore, to broaden the range of available beverages for the induction of the C-STAR system, and to establish the specificity of the synthetic biology-inspired caffeine-sensing system, C-STAR_{DB1} cells were challenged with 26 products, including Nespresso Grand Cru®, Starbucks® coffee, Red Bull®, Cuida Te® tea capsule, and CocaCola® (FIG. 24). Several Nespresso Grand Cm® capsules were also tested in their decaffeinated version as negative controls (Vivalto Lungo Decaffeinato®, Volluto Decaffeinato®, Decaffeinato Intenso®, and Arpeggio Decaffeinato®). As three of these beverage samples also have caffeinated versions (Vivalto Lungo®, Volluto®, and Arpeggio®), which are claimed by the manufacturer to be identical to the respective decaffeinated versions except for the caffeine content, they allowed us to confirm that caffeine itself upregulates gene expression and not any other of the hundreds of chemical compounds present in coffee (See Gaascht F et al., Genes Nutr., 2015, 10:51). Overall, our beverage samples covered a wide range of caffeine concentrations from 0 to 4.8 g L⁻¹. A standard dose-response curve was obtained with pure caffeine. This enabled us to convert the SEAP values from C-STAR_{DB1} cells incubated with beverage samples into caffeine concentrations.

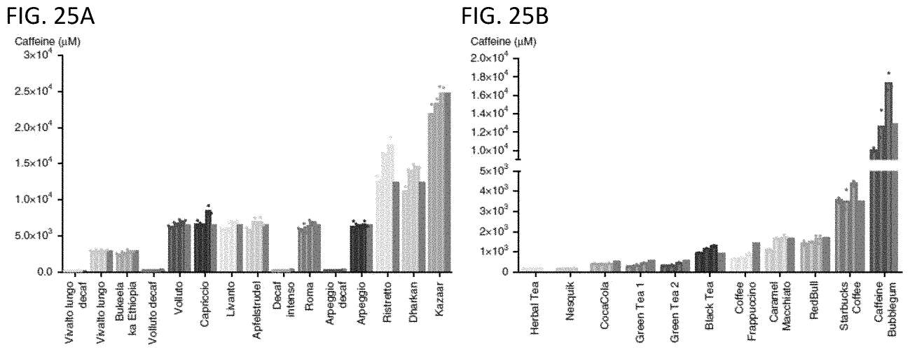

For all samples tested, caffeine concentrations indicated by the vendor corresponded well to those measured with C-STAR_{DB1} cells (FIGS. 25A-B). Decaffeinated beverage samples showed very low activation of the C-STAR system (FIGS. 25A-B). These results indicate that C-STAR reproducibly generates a dose-dependent, caffeine-specific response.

### Example 13: C-STAR Treatment for Obesity-Induced Type-2 Diabetes

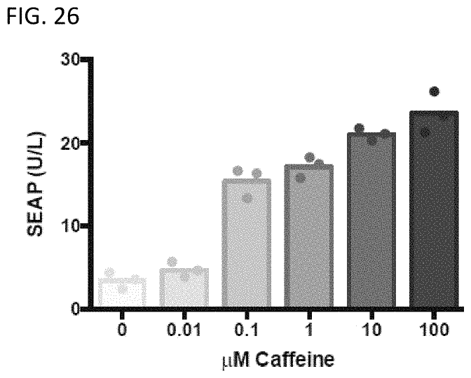

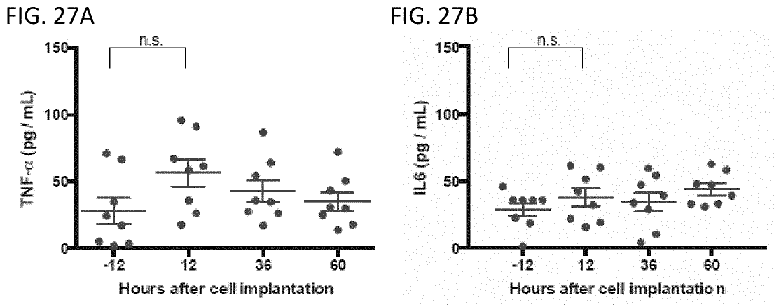

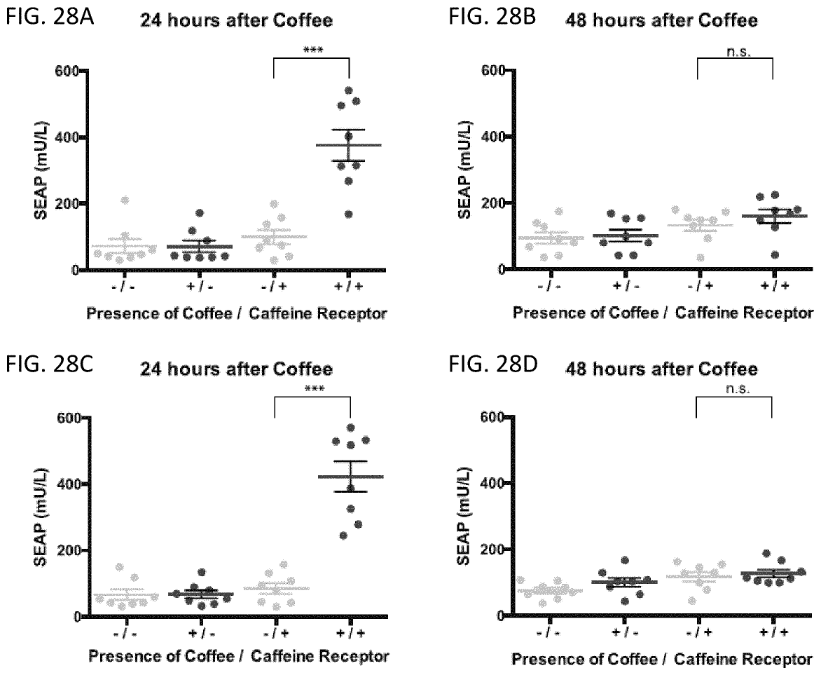

The functionality of the designed C-STAR system in vascularized microcontainers was first confirmed in vitro with pure caffeine (FIG. 26). After validating the immunoprotective function of microcapsule implants for drug delivery in vivo (FIGS. 27A-B), mice implanted with the designer cell capsules were given room temperature Volluto® coffee (Nespresso Grand Cru®), or H₂O to drink. Only mice grafted with the C-STAR system showed reversible, coffee-induced SEAP expression (FIGS. 28A-B). The same mice were restimulated a few days later and showed the same response as in the initial experiment (FIGS. 28C-D).

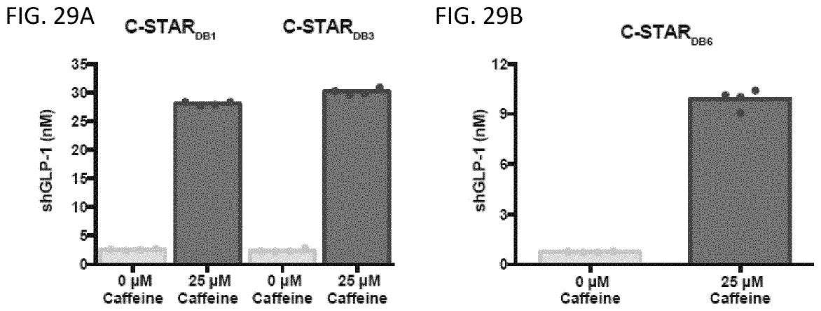

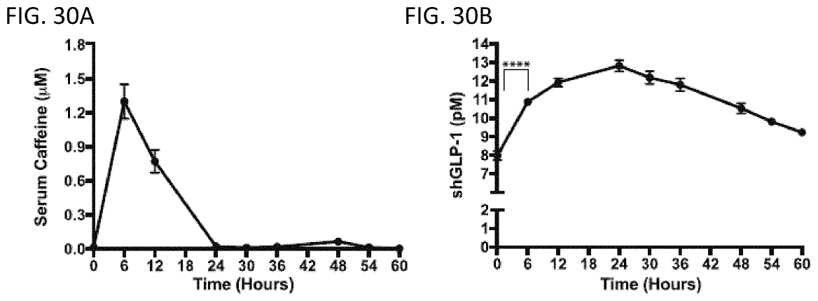

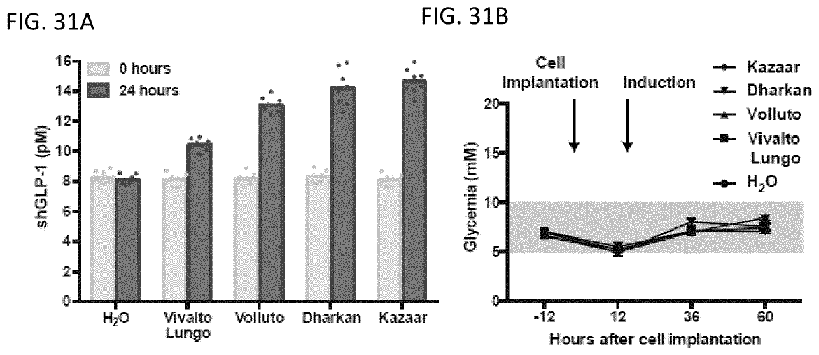

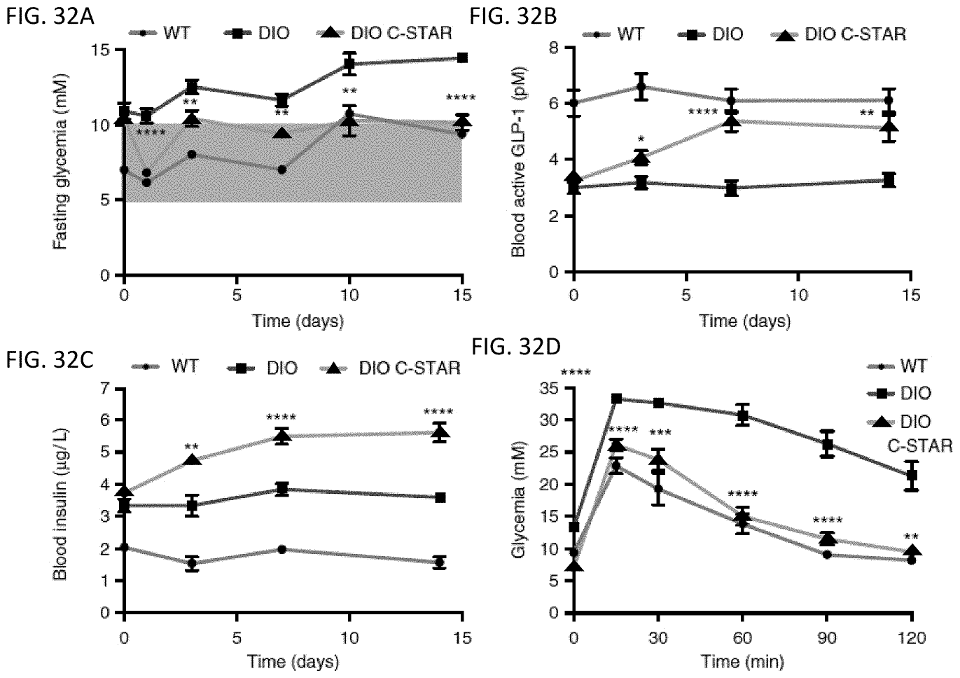

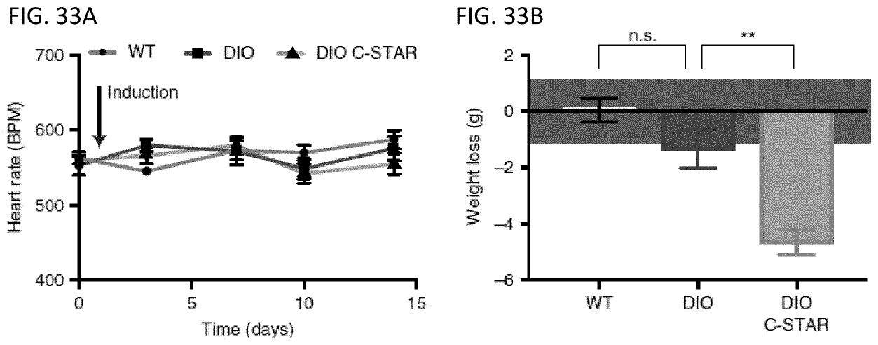

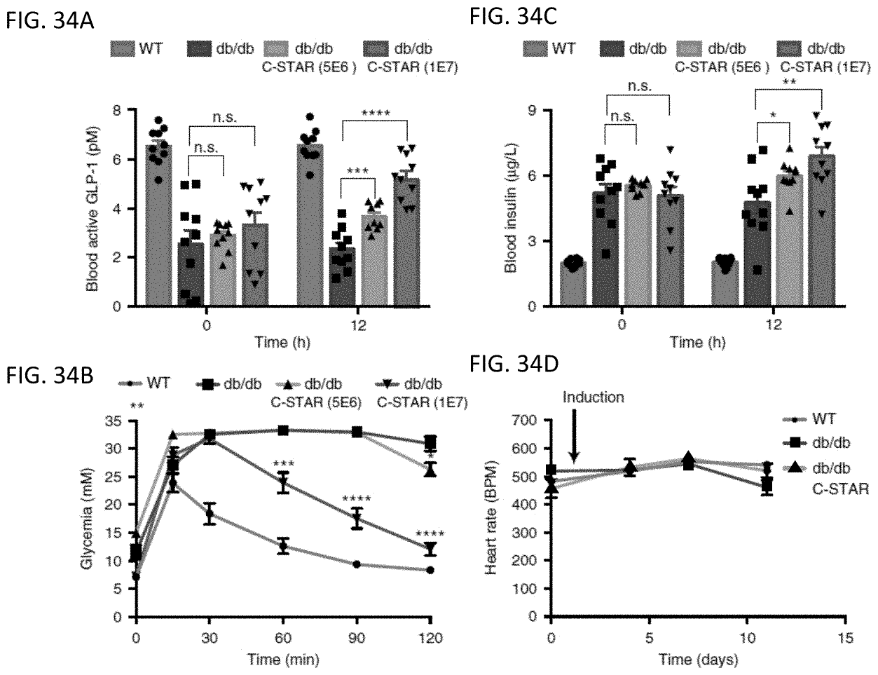

Next, in order to examine whether this system could be utilized for caffeine-induced treatment of obesity-induced T2D, we replaced the reporter gene SEAP with the gene coding for synthetic human glucagon-like peptide coupled to mouse IgG (shGLP-1, P_{STAT3}-shGLP-1-pA_{SV40}, pDB387), an engineered protein clinically licensed for the treatment of T2D (See Holz G G I V et al., Nature, 1993, 361:362-365). Experiments in vitro with the C-STAR_{DB6} cell line incorporating the resulting construct validated the caffeine-dependent expression of shGLP-1 (FIGS. 29A-B). Pharmacokinetic analyses of caffeine and shGLP-1 in mice confirmed the potential of C-STAR_{DB6} for cell-based diabetes therapy; a single oral administration of coffee resulted in a transient surge of caffeine in the bloodstream (See Xu K et al., Neuroscience, 2016 322:129-137) that was sufficient to trigger sustained shGLP-1 activity (FIGS. 30A-B). Importantly, hypoglycemic side effects were not observed following higher levels of caffeine-dependent shGLP-1 production (FIGS. 31A-B), confirming the inherent inactivity of GLP-1 in normoglycemic environments (See Doyle M E & Egan J M, Pharmacol. Ther., 2007, 113:546-593; Meier J J et al., J. Clin. Endocrinol. Metab., 2003, 88:2719-2725). Then, we examined the efficacy of these cells in two T2D mouse models with impaired insulin sensitivity. For this purpose, diet-induced obesity (DIO; see King A J F, Br. J. Pharmacol., 2012, 166:877-894) and leptin receptor-deficient (db/db; see King A J F, Br. J. Pharmacol., 2012, 166:877-894) mice were implanted intraperitoneally with capsules containing C-STAR_{DB6} cells or with control capsules containing cells equipped with only the output module pDB387 (mock). All mice received regular oral doses of Volluto® coffee. DIO mice treated with C-STAR_{DB6} cells exhibited lower fasting blood glucose values throughout a two-week experimental time course compared to the untreated control group (FIG. 32A). To demonstrate improved glycemic control in C-STAR_{DB6}-treated T2D mice, a glucose tolerance test was conducted to simulate a meal response. C-STAR_{DB6}-triggered GLP-1 production (FIG. 32B) increased the insulin levels of DIO mice (FIG. 32C) and established near-homeostatic postprandial glucose metabolism in coffee-treated diabetic mice (FIG. 32D). For db/db mice, which develop increased hyperinsulinemia compared to DIO mice (See Ye H et al., Nat. Biomed. Eng., 2016, 1:0005; FIG. 32C and FIG. 34B), GLP-1-dependent insulinotropic action (FIGS. 34A-B) and glucose tolerance (FIG. 34C) were also restored, but required a higher dose of implanted C-STAR_{DB6} cells (FIGS. 34A-C). Importantly, this coffee-triggered C-STAR_{DB6}-based diabetes therapy did not impact on the heart rate of treated animals (FIG. 33A and FIG. 34D), but reduced the body weight of diet-induced obese mice after 2 weeks (FIG. 33B).

These results indicate that the C-STAR system can be used to treat obesity-induced Type 2 diabetes in vivo.

TABLE 1
P-VALUES, T-VALUES AND DEGREES OF FREEDOM FOR TWO-SIDED T-TESTS

| | | Compared | | | Welch- |
|---|---|---|---|---|---|
| | | groups (no | | | corrected t- |
| | | adjustments | | | values |
| | | were made | | | (t) and |
| | | for multiple | Fold | | degrees of |
| Figure | Receptor | comparisons) | change | P-Value: | freedom (df) |
| FIG. 5C | JAK/STAT-GEMS_{RR120} | 0 ng vs. 1 ng RR120/mL | 5.4 | 1.848 × 10⁻⁶ | t = 8.97 |
| | | | | | df = 11.25 |
| FIG. 5C | JAK/STAT-GEMS_{RR120} | 0 ng vs. 100 ng RR120/mL | 45 | 2.543 × 10⁻¹⁰ | t = 35.39 |
| | | | | | df = 8.27 |
| FIG. 5C | MAPK-GEMS_{RR120} | 0 ng vs. 1 ng RR120/mL | 9.5 | 3.343 × 10⁻⁷ | t = 15 |
| | | | | | df = 8.11 |
| FIG. 5C | MAPK-GEMS_{RR120} | 0 ng vs. 100 ng RR120/mL | 22 | <1 × 10¹⁵ | t = 143.1 |
| | | | | | df = 9.90 |
| FIG. 8 | VEGFR2-GEMS_{RR120} (NFAT | 0 ng vs. 100 ng RR120/mL | 21 | 3.69 × 10⁻⁷ | t = 13.86 |
| | reporter) | | | | df = 8.56 |
| FIG. 8 | VEGFR2-GEMS_{RR120} | 0 ng vs. 100 ng RR120/mL | 5.2 | 1.242 × 10⁻⁷ | t = 13.16 |
| | (NF-κB reporter) | | | | df = 9.99 |
| FIG. 8 | VEGFR2-GEMS_{RR120}) (MAPK | 0 ng vs. 100 ng RR120/mL | 6.4 | 8.056 × 10⁻⁸ | t = 14.03 |
| | reporter) | | | | df = 9.82 |
| FIG. 9 | JAK/STAT-GEMS_{nicotine} | 0 μM vs. 0.1 μM Nicotine | 1.6 | 3.485 × 10⁻⁸ | t = 10.02 |
| | | | | | df = 15.56 |
| FIG. 9 | JAK/STAT-GEMS_{nicotine} | 0 μM vs. 0.33 μM Nicotine | 2.2 | 7.731 × 10⁻¹¹ | t = 16.97 |
| | | | | | df = 14.22 |
| FIG. 9 | JAK/STAT-GEMS_{nicotine} | 0 μM vs. 10 μM Nicotine | 3.4 | <1 × 10⁻¹⁵ | t = 35.82 |
| | | | | | df = 15.23 |
| FIG. 9 | MAPK-GEM_{nicotine} | 0 μM vs. 0.1 μM Nicotine | 2.5 | 4.775 × 10⁻¹² | t = 19.73 |
| | | | | | df = 14.82 |
| FIG. 9 | MAPK-GEM_{nicotine} | 0 μM vs. 0.33 μM Nicotine | 5.5 | 2.595 × 10⁻¹⁰ | t = 27.92 |
| | | | | | df = 9.33 |
| FIG. 9 | MAPK-GEM_{nicotine} | 0 μM vs. 10 μM Nicotine | 14 | 6.3 × 10³ | t = 61.27 |
| | | | | | df = 8.84 |
| FIG. 10A | JAK/STAT-GEMS_{SunTag} | 0 % vs. 0.002 % (v/v) | 6.9 | 1.246 × 10⁻⁹ | t = 21.74 |
| | | bacterial lysate (SunTag) | | | df = 8.01 |
| FIG. 10A | JAK/STAT-GEMS_{SunTag} | 0 % vs. 0.02 % (v/v) | 30 | 2.086 × 10⁻⁸ | t = 23.01 |
| | | bacterial lysate (SunTag) | | | df = 15.58 |
| FIG. 10A | MAPK-GEMS_{SunTag} | 0 % vs. 0.002 % (v/v) | 2.0 | 1.9 × 10³ | t = 23.01 |
| | | bacterial lysate (SunTag) | | | df = 15.58 |
| FIG. 10A | MAPK-GEMS_{SunTag} | 0 % vs. 0.02 % (v/v) | 2.4 | 2 × 10⁻¹⁵ | t = 30.73 |
| | | bacterial lysate (SunTag) | | | df = 15.62 |
| FIG. 11A | JAK/STAT-GEMS_{PSA} | 0 ng vs. 1 ng | 2.7 | 1.711 × 10⁻⁴ | t = 6.16 |
| | | | | | df = 8.94 |
| FIG. 11A | JAK/STAT-GEMS_{PSA} | 1 ng vs. 2 ng PSA/mL | 2.1 | 5.018 × 10⁻⁷ | t = 8.54 |
| | | | | | df = 14.45 |
| FIG. 11A | JAK/STAT-GEMS_{PSA} | 2 ng vs. 4 ng PSA/mL | 2.2 | 1.206 × 10⁻⁷ | t = 12.44 |
| | | | | | df = 10.57 |
| FIG. 11A | JAK/STAT-GEMS_{PSA} | 4 ng vs. 6 ng PSA/mL | 1.3 | 1.207 × 10⁻⁵ | t = 6.23 |
| | | | | | df = 16 |
| FIG. 11A | JAK/STAT-GEMS_{PSA} | 6 ng vs. 10 ng PSA/mL | 1.4 | 1.763 × 10⁻⁴ | t = 5.55 |
| | | | | | df = 10.94 |
| FIG. 11A | JAK/STAT-GEMS_{PSA} | 10 ng vs. 20 ng PSA/mL | 1.4 | 3.617 × 10⁻⁶ | t = 7.12 |
| | | | | | df = 14.92 |
| FIG. 11A | JAK/STAT-GEMS_{PSA} | 0 ng vs. 40 ng PSA/mL | 32 | 7.487 × 10⁻¹⁰ | t = 32.6 |
| | | | | | df = 8.07 |
| FIG. 11B | MAPK-GEMS_{PSA} | 0 ng vs. 0.1 ng PSA/mL | 2.7 | 1.019 × 10⁻⁶ | t = 11.4 |
| | | | | | df = 9.16 |
| FIG. 11B | MAPK-GEMS_{PSA} | 0.1 ng vs. 0.33 ng PSA/mL | 2.7 | 2.450 × 10⁻¹⁰ | t = 16.63 |
| | | | | | df = 13.41 |
| FIG. 11B | MAPK-GEMS_{PSA} | 0.33 ng vs. 1 ng PSA/mL | 2.2 | 7.517 × 10⁻¹⁰ | t = 17.55 |
| | | | | | df = 11.86 |
| FIG. 11B | MAPK-GEMS_{PSA} | 1 ng vs. 4 ng PSA/mL | 1.3 | 2.837 × 10⁻⁷ | t = 8.71 |
| | | | | | df = 15.11 |
| FIG. 11B | MAPK-GEMS_{PSA} | 0 ng vs. 40 ng PSA/mL | 22 | 1.536 × 10⁻⁸ | t = 22.5 |
| | | | | | df = 8.03 |
| FIG. 11C | MAPK-GEMS_{PSA} | 10% (v/v) PSA negative | 2.0 | 5.238 × 10⁻⁹ | t = 23.59 |
| | | vs. patient 1 serum | | | df = 8.45 |
| FIG. 11C | MAPK-GEMS_{PSA} | 10% (v/v) PSA negative | 4.2 | 1.018 × 10⁻⁷ | t = 14.04 |
| | | vs. patient 2 serum | | | df = 9.60 |
| FIG. 11C | MAPK-GEMS_{PSA} | 10% (v/v) PSA negative | 10 | 8.678 × 10⁻⁹ | t = 24.03 |
| | | vs. patient 3 serum | | | df = 8.06 |
| FIG. 13C | MAPK-GEMS_{RR120} | 0 ng vs. 100 ng RR120/mL | 9.3 (MAPK | 8.556 × 10⁻¹¹ | t = 34.07 |
| | (Y677F) + JAK/STAT-GEMS_{SunTag} | | reporter) | | df = 8.97 |
| FIG. 13C | MAPK-GEMS_{RR120} | 0 ng vs. 100 ng RR120/mL | 1.3 | 0.012 | t = 2.95 |
| | (Y677F) + JAK/STAT-GEMS_{SunTag} | | (STAT3 | | df = 12.02 |
| | | | reporter) | | |
| FIG. 13C | MAPK-GEMS_{RR120} | 0% vs. 0.002 % (v/v) bacterial | 1.3 | 0.038 | t = 2.36 |
| | (Y677F) + JAK/STAT-GEMS_{SunTag} | lysate (SunTag) | (MAPK | | df = 12.01 |
| | | | reporter) | | |
| FIG. 13C | MAPK-GEMS_{RR120} | 0% vs. 0.002 % (v/v) | 12.9 (STAT3 | 2.759 × 10⁻¹¹ | t = 43.83 |
| | (Y677F) + JAK/STAT-GEMS_{SunTag} | bacterial lysate (SunTag) | reporter) | | df = 8.47 |
| FIG. 13C | MAPK-GEMS_{RR120} | No inducer vs. 100 ng RR120/ | 7.4 (MAPK | 5.256 × 10⁻⁹ | t = 22.49 |
| | (Y677F) + JAK/STAT-GEMS_{SunTag} | mL + 0.002 % (v/v) | reporter) | | df = 8.69 |
| | | bacterial lysate (SunTag) | | | |
| FIG. 13C | MAPK-GEMS_{RR120} | No inducer vs. 100 ng RR120/ | 14.9 (STAT3 | 8.186 × 10⁻⁸ | t = 18.11 |
| | (Y677F) + JAK/STAT-GEMS_{SunTag} | mL + 0.002 % (v/v) | reporter) | | df = 8.057 |
| | | bacterial lysate (SunTag) | | | |
| FIG. 14 | WT WEN1.3 cells | 0 ng vs. 100 ng RR120/mL | 1.1 | 0.377 | t = 0.91 |
| | | | | | df = 14.18 |
| FIG. 14 | JAK/STAT-GEMS_{RR120} WEN1.3 | 0 ng vs. 100 ng RR120/mL | 1.5 | 3.229 × 10⁻⁵ | t = 6.09 |
| | cells | | | | df = 13.52 |
| | (polyclonal cell line) | | | | |
| FIG. 14 | MAPK-GEMS_{RR120} WEN1.3 cells | 0 ng vs. 100 ng RR120/mL | 2.4 | 5.110 × 10⁻¹⁰ | t = 13.27 |
| | (polyclonal cell line) | | | | df = 15.9 |

SEQUENCES

| SEQ | | |
|---|---|---|
| ID | | |
| NO: | SEQUENCE | DESCRIPTION |
| 8 | APSPSLPDPKFESKAALLASRGSEELLCFTQRLEDLVCFW | Minimal EpoR |
| | EEAASSGMDFNYSFSYQLEGESRKSCSLHQAPTVRGSVR | scaffold domain |
| | FWCSLPTADTSSFVPLELQVTEASGSPRYHRIIHINEVVLL | |
| | DAPAGLLARRAEEGSHVVLRWLPPPGAPMTTHIRYEVD | |
| | VSAGNRAGGTQRVEVLEGRTECVLSNLRGGTRYTFAVR | |
| | ARMAEPSFSGFWSAWSEPASLLTASDLDPLILTLSLILVLI | |
| | SLLLTVLALLS | |
| 9 | APSPSLPDPKFESKAALLASRGSEELLCFTQRLEDLVCFW | Minimal EpoR |
| | EEAASSGMDFNYSFSYQLEGESRKSCSLHQAPTVRGSVR | extracellular |
| | FWCSLPTADTSSFVPLELQVTEASGSPRYHRIIHINEVVLL | domain |
| | DAPAGLLARRAEEGSHVVLRWLPPPGAPMTTHIRYEVD | |
| | VSAGNRAGGTQRVEVLEGRTECVLSNLRGGTRYTFAVR | |
| | ARMAEPSFSGFWSAWSEPASLLTASDLDP | |
| 10 | LILTLSLILVLISLLLTVLALLS | Minimal EpoR |
| | | transmembrane |
| | | domain |
| 11 | APSPSLPDPKFESKAALLASRGSEELLCFTQRLEDLVCFW | Minimal EpoRₘ |
| | EEAASSGMDFNYSFSYQLEGESRKSCSLHQAPTVRGSVR | scaffold domain |
| | FWCSLPTADTSSAVPLELQVTEASGSPRYHRIIHINEVVL | with F93A |
| | LDAPAGLLARRAEEGSHVVLRWLPPPGAPMTTHIRYEV | mutation |
| | DVSAGNRAGGTQRVEVLEGRTECVLSNLRGGTRYTFAV | |
| | RARMAEPSFSGFWSAWSEPASLLTASDLDPLILTLSLILV | |
| | LISLLLTVLALLS | |
| 12 | MASRILWHEMWHEGLEEASRLYFGERNVKGMFEVLEPL | FRB |
| | HAMMERGPQTLKETSFNQAYGRDLMEAQEWCRKYMK | |
| | SGNVKDLLQAWDLYYHVFRRISK | |
| 13 | MGVQVETISPGDGRTFPKRGQTCVVHYTGMLEDGKKFD | FKBP |
| | SSRDRNKPFKFMLGKQEVIRGWEEGVAQMSVGQRAKLT | |
| | ISPDYAYGATGHPGIIPPHATLVFDVELLKLE | |
| 14 | VQLQESGGGLVQAGDSLKLSCEASGDSIGTYVIGWFRQA | VHH_{A52} |
| | PGKERIYLATIGRNLVGPSDFYTRYADSVKGRFAVSRDN | |
| | AKNTVNLQMNSLKPEDTAVYYCAAKTTTWGGNDPNN | |
| | WNYWGQGTQVTV | |
| 15 | DIVMTQSPSSLSASVGDRVTITCRSSTGAVTTSNYASWV | SCFv_{αGCN4} |
| | QEKPGKLFKGLIGGTNNRAPGVPSRFSGSLIGDKATLTIS | |
| | SLQPEDFATYFCALWYSNHWVFGQGTKVELKRGGGGS | |
| | GGGGSGGGGSSGGGSEVKLLESGGGLVQPGGSLKLSCA | |
| | VSGFSLTDYGVNWVRQAPGRGLEWIGVIWGDGITDYNS | |
| | ALKDRFIISKDNGKNTVYLQMSKVRSDDTALYYCVTGL | |
| | FDYWGQGTLVTVS | |
| 16 | DIVMTQTAPSVFVTPGESVSISCRSSKSLLHSNGNTYLYW | scFv_{5d311} |
| | FLQRPGQSPQLLIYRMSNLASGVPDRFSGSGSGTDFTLRI | |
| | SRVEAEDVGVYYCMQHLEYPVTFGAGTKVEIKRGGGGS | |
| | GGGGSGGGGSGGGGSQVQLQQSGPELVKPGASVKISCK | |
| | VSGYAISSSWMNWVKQRPGHGLEWIGRIYPGDGDTKYN | |
| | GKFKDKATLTVDKSSSTAYMQLSSLTSVDSAVYFCARD | |
| | GYRYYFDYWGQGTSVTVSS | |
| 17 | DIVLTQSPPSLAVSLGQRATISCRASESIDLYGFTFMHWY | scFv₅ₐ₅ |
| | QQKPGQPPKILIYRASNLESGIPARFSGSGSRTDFTLTINP | |
| | VEADDVATYYCQQTHEDPYTFGGGTKLEIKRGGGGSGG | |
| | GGSGGGGSGGGGSQVQLQQSGAELAKPGASVKMSCKT | |
| | SGYSFSSYWMHWVKQRPGQGLEWIGYINPSTGYTENNQ | |
| | KFKDKVTLTADKSSNTAYMQLNSLTSEDSAVYYCARSG | |
| | RLYFDVWGAGTTVTVSS | |
| 18 | DIVLTQSPASLAVSLGQRATISCKASQSVDFDGDSYMNW | ScFv_{8g8f5} |
| | YQQKPGQPPKLLIFAASNLASGIPARLSGSGSGTDFTLNI | |
| | QPVEEEDAATYYCQQSNEDPYTFGGGTKLEIKGGGGSG | |
| | GGGSGGGGSGGGGSQVQLQQSGDDLVKPGASVKLSCK | |
| | ASGYTFTTYYINWMRQRPGQGLEWIGRIAPASGTTYSSE | |
| | MFKDKATLTVDTSSNTAYIQLSSLSSEDSAVYFCARADY | |
| | GFNSGEAMDYWGQGTSVTVSS | |
| 19 | QSELTQPPSASGTPGQRVTISCSGSSSNIGSNYVYWYQQL | Nic12_{VL} |
| | PGTAPKLLIYRNNQRPSGVPDRFSGSKSGTSASLAISGLR | |
| | SEDEADYYCAAWDDSLSAWVFGGGTQLDILG | |
| 20 | QMQLLESGPGLVKPSETLSLTCTVSGGSIWGWIRQPPGK | Nic12_{VH} |
| | GLEWIGSIYSSGSTYYNPSLKSRVTTSVDTSKNQFSLRLS | |
| | SVTAADTAVYYCVAWFGDLLSLKGVELWGQGTLVTVS | |
| 21 | GSQVQLVESGGGLVQAGGSLRLSCTASGRTGTIYSMAW | aCaffVHH |
| | FRQAPGKEREFLATVGWSSGITYYMDSVKGRFTISRDKG | |
| | KNTVYLQMDSLKPEDTAVYYCTATRAYSVGYDYWGQG | |
| | TQVTVSS | |
| 22 | NKRDLIKKHIWPNVPDPSKSHIAQWSPHTPPRHNFNSKD | IL-6RBᵢₙₜ |
| | QMYSDGNFTDVSVVEIEANDKKPFPEDLKSLDLFKKEKI | |
| | NTEGHSSGIGGSSCMSSSRPSISSSDENESSQNTSSTVQYS | |
| | TVVHSGYRHQVPSVQVFSRSESTQPLLDSEERPEDLQLV | |
| | DHVDGGDGILPRQQYFKQNCSQHESSPDISHFERSKQVS | |
| | SVNEEDFVRLKQQISDHISQSCGSGQMKMFQEVSAADAF | |
| | GPGTEGQVERFETVGMEAATDEGMPKSYLPQTVRQGGY | |
| | MPQ | |
| 23 | NKRDLIKKHIWPNVPDPSKSHIAQWSPHTPPRHNFNSKD | IL-6RBₘ with |
| | QMYSDGNFTDVSVVEIEANDKKPFPEDLKSLDLFKKEKI | Y759A mutation |
| | NTEGHSSGIGGSSCMSSSRPSISSSDENESSQNTSSTVQAS | |
| | TVVHSGYRHQVPSVQVFSRSESTQPLLDSEERPEDLQLV | |
| | DHVDGGDGILPRQQYFKQNCSQHESSPDISHFERSKQVS | |
| | SVNEEDFVRLKQQISDHISQSCGSGQMKMFQEVSAADAF | |
| | GPGTEGQVERFETVGMEAATDEGMPKSYLPQTVRQGGY | |
| | MPQ | |
| 24 | LRTVKRANGGELKTGYLSIVMDPDELPLDEHCERLPYDA | VEGFR2ᵢₙₜ |
| | SKWEFPRDRLKLGKPLGRGAFGQVIEADAFGIDKTATCR | |
| | TVAVKMLKEGATHSEHRALMSELKILIHIGHHLNVVNLL | |
| | GACTKPGGPLMVIVEFCKFGNLSTYLRSKRNEFVPYKTK | |
| | GARFRQGKDYVGAIPVDLKRRLDSITSSQSSASSGFVEEK | |
| | SLSDVEEEEAPEDLYKDFLTLEHLICYSFQVAKGMEFLAS | |
| | RKCIHRDLAARNILLSEKNVVKICDFGLARDIYKDPDYV | |
| | RKGDARLPLKWMAPETIFDRVYTIQSDVWSFGVLLWEIF | |
| | SLGASPYPGVKIDEEFCRRLKEGTRMRAPDYTTPEMYQT | |
| | MLDCWHGEPSQRPTFSELVEHLGNLLQANAQQDGKDYI | |
| | VLPISETLSMEEDSGLSLPTSPVSCMEEEEVCDPKFHYDN | |
| | TAGISQYLQNSKRKSRPVSVKTFEDIPLEEPEVKVIPDDN | |
| | QTDSGMVLASEELKTLEDRTKLSPSFGGMVPSKSRESVA | |
| | SEGSNQTSGYQSGYHSDDTDTTVYSSEEAELLKLIEIGVQ | |
| | TGSTAQILQPDSGTTLSSPPV | |
| 25 | MKSGTKKSDFHSQMAVHKLAKSIPLRRQVTVSADSSAS | FGFR1ᵢₙₜ |
| | MNSGVLLVRPSRLSSSGTPMLAGVSEYELPEDPRWELPR | |
| | DRLVLGKPLGEGCFGQVVLAEAIGLDKDKPNRVTKVAV | |
| | KMLKSDATEKDLSDLISEMEMMKMIGKHKNIINLLGAC | |
| | TQDGPLYVIVEYASKGNLREYLQARRPPGLEYCYNPSHN | |
| | PEEQLSSKDLVSCAYQVARGMEYLASKKCIHRDLAARN | |
| | VLVTEDNVMKIADFGLARDIHHIDYYKKTTNGRLPVKW | |
| | MAPEALFDRIYTHQSDVWSFGVLLWEIFTLGGSPYPGVP | |
| | VEELFKLLKEGHRMDKPSNCTNELYMMMRDCWHAVPS | |
| | QRPTFKQLVEDLDRIVALTSNQEYLDLSIPLDQYSPSFPD | |
| | TRSSTCSSGEDSVFSHEPLPEEPCLPRHPTQLANSGLKRR | |

1. A chimeric ligand receptor comprising a receptor subunit, wherein the receptor subunit comprises a scaffold domain;
wherein the scaffold domain comprises an extracellular domain and a transmembrane domain;
wherein the extracellular domain is operably linked to a ligand binding domain;
wherein the transmembrane domain is operably linked to an intracellular signaling domain;
wherein the receptor subunit multimerizes via its scaffold domain in the presence of one or more additional receptor subunits; and
wherein the multimerized receptor subunits undergo a conformational reorganization upon ligand binding to the chimeric ligand receptor.

2. The chimeric ligand receptor of claim 1, further comprising one or more additional receptor subunits, wherein each additional receptor subunit comprises a scaffold domain;
wherein the scaffold domain of each additional receptor subunit comprises an extracellular domain and a transmembrane domain;
wherein the extracellular domain of each additional receptor subunit is operably linked to a ligand binding domain; and
wherein the transmembrane domain of each additional receptor subunit is operably linked to an intracellular signaling domain;
wherein the receptor subunits multimerize via their scaffold domains to form the chimeric ligand receptor; and
wherein the multimerized receptor subunits undergo a conformational reorganization upon ligand binding to the chimeric receptor.

3. (canceled)

4. (canceled)

5. The chimeric ligand receptor of claim 2, wherein the chimeric ligand binding domains of each receptor subunit bind the same ligand.

6-8. (canceled)

9. The chimeric ligand receptor of claim 2, wherein the multimerized receptor subunits comprise a dimer.

10. The chimeric ligand receptor of claim 9, wherein the conformational reorganization comprises a rotation of each scaffold domain around its own axis, and wherein the conformational reorganization activates the intracellular signaling domains of each receptor subunit.

11. (canceled)

12. (canceled)

13. The chimeric ligand receptor of claim 1, wherein the scaffold domain comprises the extracellular domain and transmembrane domain of an erythropoietin receptor (EpoR), wherein the scaffold domain is inert to erythropoietin, the ligand binding domain does not bind erythropoietin, and the intracellular signaling domain does not comprise an endogenous erythropoietin receptor (EpoR) intracellular signaling domain.

14. (canceled)

15. (canceled)

16. The chimeric ligand receptor of claim 1, wherein the scaffold domain comprises an extracellular domain and transmembrane domain comprising an amino acid sequence having 90% or greater sequence identity to SEQ ID NO: 8.

17. (canceled)

18. The chimeric ligand receptor of claim 1, wherein the extracellular domain comprises an F93A amino acid substitution.

19. The chimeric ligand receptor of claim 1, wherein one or more additional amino acid residues are inserted adjacent to or within the transmembrane domain, wherein the one or more additional amino acid residues comprise one, two, three, or four additional alanine residues.

20-24. (canceled)

25. The chimeric ligand receptor of claim 1, wherein the ligand binding domain is linked to the extracellular domain through an extracellular linker region that comprises one or more amino acid residues, wherein the one or more amino acid residues comprise amino acids residues Serine-Glycine-Glutamic acid-Phenylalanine (SEQ ID NO: 26).

26-28. (canceled)

29. The chimeric ligand receptor of claim 1, wherein the ligand binding domain binds to a soluble ligand selected from the group consisting of a protein complex, a protein, a peptide, a nucleic acid, a small molecule, and a chemical agent.

30-43. (canceled)

44. The chimeric ligand receptor of claim 1, wherein the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway.

45. The chimeric ligand receptor of claim 1, wherein the intracellular signaling domain is selected from the group consisting of an intracellular signal transduction domain of IL-6RB (interleukin 6 receptor B), an intracellular signal transduction domain of FGFR1 (fibroblast growth factor receptor 1), and an intracellular signal transduction domain of VEGFR2 (vascular endothelial growth factor receptor 2).

46-49. (canceled)

50. The chimeric ligand receptor of claim 1, wherein the intracellular signaling domain comprises one or more modifications that modulate signaling activity of the intracellular signaling domain, wherein the one or more modifications are one or more amino acid substitutions.

51-59. (canceled)

60. An isolated polynucleotide or a set of isolated polynucleotides encoding the chimeric ligand receptor of claim 1.

61-120. (canceled)

121. A vector or a set of vectors comprising the polynucleotide or set of polynucleotides of claim 60.

122. (canceled)

123. A genetically engineered cell expressing the chimeric ligand receptor of claim 1.

124. The genetically engineered cell of claim 123, wherein the cell further comprises an engineered transgene, wherein the transgene comprises a synthetic promoter operably linked to a polynucleotide comprising a nucleic acid sequence encoding a target product.

125. The genetically engineered cell of claim 124, wherein the synthetic promoter is responsive to intracellular signaling from the chimeric ligand receptor, and wherein the target product is selected from the group consisting of a therapeutic molecule, a prophylactic molecule, and a diagnostic molecule.

126-143. (canceled)

144. A method comprising contacting the genetically engineered cell of claim 123 with a biological tissue or biological fluid.

145-156. (canceled)


SEQUENCE LISTING (28 sequences)

SEQ ID NO: 1 - protein, 507 aa, Mus musculus
MDKLRVPLWPRVGPLCLLLAGAAWAPSPSLPDPKFESKAALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSHRRTLQQKIWPGIPSPESEFEGLFTTHKGNFQLWLLQRDGCLWWSPGSSFPEDPPAHLEVLSEPRWAVTQAGDPGADDEGPLLEPVGSEHAQDTYLVLDKWLLPRTPCSENLSGPGGSVDPVTMDEASETSSCPSDLASKPRPEGTSPSSFEYTILDPSSQLLCPRALPPELPPTPPHLKYLYLVVSDSGISTDYSSGGSQGVHGDSSDGPYSHPYENSLVPDSEPLHPGYVACS

SEQ ID NO: 2 - protein, 483 aa, Mus musculus
APSPSLPDPKFESKAALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSHRRTLQQKIWPGIPSPESEFEGLFTTHKGNFQLWLLQRDGCLWWSPGSSFPEDPPAHLEVLSEPRWAVTQAGDPGADDEGPLLEPVGSEHAQDTYLVLDKWLLPRTPCSENLSGPGGSVDPVTMDEASETSSCPSDLASKPRPEGTSPSSFEYTILDPSSQLLCPRALPPELPPTPPHLKYLYLVVSDSGISTDYSSGGSQGVHGDSSDGPYSHPYENSLVPDSEPLHPGYVACS

SEQ ID NO: 3 - protein, 265 aa, Artificial Sequence
  Description of Artificial Sequence Synthetic polypeptide
MDKLRVPLWPRVGPLCLLLAGAAWAPSPSLPDPKFESKAALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASGEALVPRGAGGAGPNTRQTP

SEQ ID NO: 4 - protein, 508 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 5 - protein, 484 aa, Homo sapiens
APPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 6 - protein, 241 aa, Artificial Sequence
  Description of Artificial Sequence Synthetic polypeptide
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRGTVFLSPDWLSSTRARPHVIYFCLLRVPRPDSAPRWRSWRAAPSVC

SEQ ID NO: 7 - protein, 328 aa, Artificial Sequence
  Description of Artificial Sequence Synthetic polypeptide
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQVGGLVVPSVPGLPCFLQPNCRPL

SEQ ID NO: 8 - protein, 248 aa, Unknown
  Description of Unknown Minimal EpoR scaffold domain sequence
APSPSLPDPKFESKAALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLS

SEQ ID NO: 9 - protein, 225 aa, Unknown
  Description of Unknown Minimal EpoR extracellular domain sequence
APSPSLPDPKFESKAALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDP

SEQ ID NO: 10 - protein, 23 aa, Unknown
  Description of Unknown Minimal EpoR transmembrane domain sequence
LILTLSLILVLISLLLTVLALLS

SEQ ID NO: 11 - protein, 248 aa, Artificial Sequence
  Description of Artificial Sequence Synthetic polypeptide
APSPSLPDPKFESKAALLASRGSEELLCFTQRLEDLVCFWEEAASSGMDFNYSFSYQLEGESRKSCSLHQAPTVRGSVRFWCSLPTADTSSAVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLS

SEQ ID NO: 12 - protein, 97 aa, Artificial Sequence
  Description of Artificial Sequence Synthetic polypeptide
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 13 - protein, 108 aa, Artificial Sequence
  Description of Artificial Sequence Synthetic polypeptide
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 14 - protein, 126 aa, Artificial Sequence
  Description of Artificial Sequence Synthetic polypeptide
VQLQESGGGLVQAGDSLKLSCEASGDSIGTYVIGWFRQAPGKERIYLATIGRNLVGPSDFYTRYADSVKGRFAVSRDNAKNTVNLQMNSLKPEDTAVYYCAAKTTTWGGNDPNNWNYWGQGTQVTV

SEQ ID NO: 15 - protein, 243 aa, Artificial Sequence
  Description of Artificial Sequence Synthetic polypeptide
DIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVS

SEQ ID NO: 16 - protein, 251 aa, Artificial Sequence
  Description of Artificial Sequence Synthetic polypeptide
DIVMTQTAPSVFVTPGESVSISCRSSKSLLHSNGNTYLYWFLQRPGQSPQLLIYRMSNLASGVPDRFSGSGSGTDFTLRISRVEAEDVGVYYCMQHLEYPVTFGAGTKVEIKRGGGGSGGGGSGGGGSGGGGSQVQLQQSGPELVKPGASVKISCKVSGYAISSSWMNWVKQRPGHGLEWIGRIYPGDGDTKYNGKFKDKATLTVDKSSSTAYMQLSSLTSVDSAVYFCARDGYRYYFDYWGQGTSVTVSS

SEQ ID NO: 17 - protein, 249 aa, Artificial Sequence
  Description of Artificial Sequence Synthetic polypeptide
DIVLTQSPPSLAVSLGQRATISCRASESIDLYGFTFMHWYQQKPGQPPKILIYRASNLESGIPARFSGSGSRTDFTLTINPVEADDVATYYCQQTHEDPYTFGGGTKLEIKRGGGGSGGGGSGGGGSGGGGSQVQLQQSGAELAKPGASVKMSCKTSGYSFSSYWMHWVKQRPGQGLEWIGYINPSTGYTENNQKFKDKVTLTADKSSNTAYMQLNSLTSEDSAVYYCARSGRLYFDVWGAGTTVTVSS

SEQ ID NO: 18 - protein, 253 aa, Artificial Sequence
  Description of Artificial Sequence Synthetic polypeptide
DIVLTQSPASLAVSLGQRATISCKASQSVDFDGDSYMNWYQQKPGQPPKLLIFAASNLASGIPARLSGSGSGTDFTLNIQPVEEEDAATYYCQQSNEDPYTFGGGTKLEIKGGGGSGGGGSGGGGSGGGGSQVQLQQSGDDLVKPGASVKLSCKASGYTFTTYYINWMRQRPGQGLEWIGRIAPASGTTYSSEMFKDKATLTVDTSSNTAYIQLSSLSSEDSAVYFCARADYGFNSGEAMDYWGQGTSVTVSS

SEQ ID NO: 19 - protein, 111 aa, Artificial Sequence
  Description of Artificial Sequence Synthetic polypeptide
QSELTQPPSASGTPGQRVTISCSGSSSNIGSNYVYWYQQLPGTAPKLLIYRNNQRPSGVPDRFSGSKSGTSASLAISGLRSEDEADYYCAAWDDSLSAWVFGGGTQLDILG

SEQ ID NO: 20 - protein, 116 aa, Artificial Sequence
  Description of Artificial Sequence Synthetic polypeptide
QMQLLESGPGLVKPSETLSLTCTVSGGSIWGWIRQPPGKGLEWIGSIYSSGSTYYNPSLKSRVTTSVDTSKNQFSLRLSSVTAADTAVYYCVAWFGDLLSLKGVELWGQGTLVTVS

SEQ ID NO: 21 - protein, 121 aa, Artificial Sequence
  Description of Artificial Sequence Synthetic polypeptide
GSQVQLVESGGGLVQAGGSLRLSCTASGRTGTIYSMAWFRQAPGKEREFLATVGWSSGITYYMDSVKGRFTISRDKGKNTVYLQMDSLKPEDTAVYYCTATRAYSVGYDYWGQGTQVTVSS

SEQ ID NO: 22 - protein, 277 aa, Artificial Sequence
  Description of Artificial Sequence Synthetic polypeptide
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 23 - protein, 277 aa, Artificial Sequence
  Description of Artificial Sequence Synthetic polypeptide
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQASTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 24 - protein, 571 aa, Artificial Sequence
  Description of Artificial Sequence Synthetic polypeptide
LRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 25 - protein, 424 aa, Artificial Sequence
  Description of Artificial Sequence Synthetic polypeptide
MKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSIPLDQYSPSFPDTRSSTCSSGEDSVFSHEPLPEEPCLPRHPTQLANSGLKRR

SEQ ID NO: 26 - protein, 4 aa, Artificial Sequence
  Description of Artificial Sequence Synthetic peptide
SGEF

SEQ ID NO: 27 - protein, 20 aa, Artificial Sequence
  Description of Artificial Sequence Synthetic peptide
GGGGSGGGGSGGGGSGGGGS

SEQ ID NO: 28 - protein, 4 aa, Artificial Sequence
  Description of Artificial Sequence Synthetic peptide
AAAA